(19) United States
(12) Patent Application Publication
(10) Pub. No.: US 2019/0055553 A1
(43) Pub. Date: Feb. 21, 2019

(54) METHODS FOR IDENTIFYING AND TARGETING NON-CODING RNA SCAFFOLDS

(71) Applicant: TRANSLATE BIO MA, INC., Lexington, MA (US)

(72) Inventors: James BARSOUM, Lexington, MA (US); Caroline WOO, Newton, MA (US)

(73) Assignee: TRANSLATE BIO MA, INC., Lexington, MA (US)

(21) Appl. No.: 15/768,579

(22) PCT Filed: Oct. 14, 2016
(86) PCT No.: PCT/US16/57076

§ 371 Date: Apr. 16, 2018

Related U.S. Application Data
(60) Provisional application No. 62/369,729, filed on Aug. 1, 2016.
(60) Provisional application No. 62/343,335, filed on May 31, 2016.
(60) Provisional application No. 62/242,863, filed on Oct. 16, 2015.

(51) Int. Cl. C12N 15/113

(52) U.S. Cl. CPC C12N 15/113; C12N 2310/113

(57) ABSTRACT

Aspects of the disclosure provide steric-blocking oligonucleotide-based methods of modulating expression of target genes, e.g., by targeting non-coding RNA scaffolds.

### CROSS-REFERENCE TO RELATED APPLICATIONS

This application claims the benefit under 35 U.S.C. § 119(e) of U.S. Provisional Application No. U.S. 62/242,863, filed on Oct. 16, 2015, U.S. Provisional Application No. U.S. 62/343,335, filed on May 31, 2016, and U.S. Provisional Application No. U.S. 62/369,729, filed on Aug. 1, 2016, the contents of each of which are incorporated herein by reference in its entirety.

### FIELD OF THE DISCLOSURE

The disclosure relates to methods of targeting non-coding RNAs.

### BACKGROUND OF THE DISCLOSURE

Non-coding RNAs are functional RNA molecules that do not encode proteins. Examples of non-coding RNAs include transfer RNAs, ribosomal RNAs, snoRNAs, microRNAs, and long ncRNAs, among others. Non-coding RNAs have been identified that regulate gene expression at different levels, including transcriptional and post-transcriptional levels.

### SUMMARY OF THE DISCLOSURE

Aspects of the disclosure relate to methods and compositions for modulating gene expression that involve targeting of non-coding RNAs. In some aspects, the disclosure relates to a discovery that certain non-coding RNAs function as scaffolds that recruit activators and/or repressors to genes to control gene expression. In some embodiments, it has been found that non-coding RNA scaffolds have one or more distinct regions that interact with activators and one or more distinct regions that interact with repressors. Thus, in some embodiments, non-coding RNA scaffolds have different effects on gene expression, depending on whether they interact with activators or repressors. In some embodiments, it has been found that non-coding RNA scaffolds are expressed from the same chromosomal locus as their respective target genes. Thus, in some embodiments, a non-coding RNA scaffold that controls expression of a target gene can be readily identified based on the proximity of its coding sequence to the coding sequence of the target gene. In some embodiments, non-coding RNA scaffolds contain a nucleotide sequence encoded in the antisense strand of a chromosomal locus of a target gene. However, in some embodiments, non-coding RNA scaffolds contain a nucleotide sequence encoded in the sense strand of a chromosomal locus of a target gene.

Aspects of the disclosure relate to the discovery that oligonucleotides complementary with interaction regions of non-coding RNA scaffolds can be used to block interactions between the non-coding RNA scaffolds and activators or repressors (or both) to alter gene expression. In some embodiments, expression of a target gene can be increased by selectively blocking interaction of repressors (e.g., PRC2) with the non-coding RNA scaffolds. However, in some embodiments, expression of a target gene can be decreased by selectively blocking interaction of activators (e.g., histone modifying enzymes associated with active chromatin, e.g., histone lysine methyltransferases) with the non-coding RNA scaffolds.

In some embodiments, recruitment of a repressor to a gene via a non-coding RNA scaffold can be blocked or inhibited by using a steric-blocking oligonucleotide that sterically blocks or inhibits interaction of the repressor with the non-coding RNA scaffold without inducing degradation of the target non-coding RNA scaffold. In such embodiments, the non-coding RNA scaffold remains intact and capable of interacting with, and thereby recruiting, the activator to the target gene to bring about increased expression of the target gene. Similarly, in some embodiments, recruitment of an activator to a gene via a non-coding RNA scaffold can be blocked or inhibited by using a steric-blocking oligonucleotide that sterically blocks interaction of the activator with the non-coding RNA scaffold without inducing degradation of the target non-coding RNA scaffold. In some embodiments, the non-coding RNA scaffold remains intact and capable of interacting with, and thereby recruiting, the repressor to the target gene to bring about decreased expression of the target gene. Accordingly, in some embodiments, it is undesirable to use an oligonucleotide (such as, e.g., a gapmer oligonucleotide) that results in degradation of the target non-coding RNA scaffold because by degrading the RNA scaffold repressors and/or activators will not be recruited to the gene.

Accordingly, in some aspects of the disclosure, methods are provided for preparing an oligonucleotide that modulates expression of a target gene. In some embodiments, the methods involve determining that a non-coding RNA scaffold has a first interaction region that interacts with a repressor and a second interaction region that interacts with an activator. In some embodiments, the methods comprise preparing a steric-blocking oligonucleotide having a region of complementarity that is complementary with the first interaction region. In some embodiments, the methods comprise preparing a steric-blocking oligonucleotide having a region of complementarity that is complementary with the second interaction region.

In some embodiments, methods provided herein involve determining that a non-coding RNA scaffold interacts with an activator of the target gene and a repressor of the target gene; identifying an interaction region of the non-coding RNA that interacts with either the activator or the repressor, but not both; and preparing a steric-blocking oligonucleotide having a region of complementarity that is complementary with the interaction region.

According to some aspects of the disclosure, methods are provided herein for modulating expression of a target gene in a cell. In some embodiments, the methods involve delivering to the cell an effective amount of a steric-blocking oligonucleotide, in which the cell expresses a non-coding RNA scaffold. In some embodiments, prior to delivering the steric-blocking oligonucleotide it has been determined that the non-coding RNA scaffold has a first interaction region that interacts with a repressor and a second interaction region that interacts with an activator. In some embodiments, the steric-blocking oligonucleotide has a region of complementarity that is complementary with the first interaction region. However, in some embodiments, the steric-blocking oligonucleotide has a region of complementarity that is complementary with the second interaction region.

According to some aspects of the disclosure, methods are provided for modulating expression of a target gene in a cell, in which it has been determined that a non-coding RNA interacts with both an activator of the target gene and a repressor of a target gene. In some embodiments, the methods involve delivering to the cell a steric-blocking oligonucleotide having a region of complementarity that is complementary with a region of the non-coding RNA that interacts with either the activator or the repressor, but not both.

According to some aspects of the disclosure, methods are provided for increasing expression of a target gene in a cell, in which the cell expresses a non-coding RNA scaffold associated with the target gene. In some embodiments, the methods involve delivering to the cell an effective amount of a steric-blocking oligonucleotide. In some embodiments, prior to delivering the steric-blocking oligonucleotide it has been determined that the non-coding RNA scaffold has a first interaction region that interacts with a repressor and a second interaction region that interacts with an activator. In some embodiments, the steric-blocking oligonucleotide has a region of complementarity that is complementary with the first interaction region. In some embodiments, the steric-blocking oligonucleotide blocks interaction of the repressor with the first interaction region. In some embodiments, the methods comprise delivering to the cell a steric-blocking oligonucleotide having a region of complementarity that is complementary with a region of the non-coding RNA scaffold that interacts with the repressor, in which displacement of the repressor from the non-coding RNA, but not the activator, indicates effectiveness of the steric-blocking oligonucleotide.

According to some aspects of the disclosure, methods are provided for decreasing expression of a target gene in a cell, in which the cell expresses a non-coding RNA scaffold associated with the target gene. In some embodiments, the methods involve delivering to the cell an effective amount of a steric-blocking oligonucleotide. In some embodiments, prior to delivering the steric-blocking oligonucleotide it has been determined that the non-coding RNA scaffold has a first interaction region that interacts with a repressor and a second interaction region that interacts with an activator. In some embodiments, the steric-blocking oligonucleotide has a region of complementarity that is complementary with the second interaction region. In some embodiments, the steric-blocking oligonucleotide blocks interaction of the activator with the second interaction region. In some embodiments, the methods comprise delivering to the cell a steric-blocking oligonucleotide having a region of complementarity that is complementary with a region of the non-coding RNA scaffold that interacts with the activator, in which displacement of the activator from the non-coding RNA, hut not the repressor, indicates effectiveness of the steric-blocking oligonucleotide.

In some embodiments of methods provided herein, the steric-blocking oligonucleotide is complementary with an interaction region of the non-coding RNA scaffold that interacts with a repressor and selectively inhibits interaction of the repressor with the non-coding RNA scaffold. In some embodiments of methods provided herein, the steric-blocking oligonucleotide is complementary with an interaction region of the non-coding RNA scaffold that interacts with an activator and selectively inhibits interaction of the activator with the non-coding RNA scaffold. In some embodiments, the region of complementarity is at least 7 contiguous nucleotides in length. In some embodiments, the region of complementarity is in a range of 7 to 20 nucleotides in length. In some embodiments, the steric-blocking oligonucleotide is a mixmer.

In some embodiments, the target gene is an SMN gene or other gene having an associated non-coding RNA scaffold having an interaction region that interacts with a repressor and an interaction region that interacts with an activator. In some embodiments, the repressor is a Polycomb Repressive Complex 1 or 2 or a subunit thereof. In some embodiments, the repressor is SUZ12, EZH2, EED, AEBP2, JARID2, PCL, RhAp46/48, or EZH1. In some embodiments, the activator is a histone methyltransferase. In some embodiments, the activator is SETD2. In some embodiments, the non-coding RNA scaffold is expressed from a chromosomal locus containing a target gene. In some embodiments, the cell is in vivo. In some embodiments, the cell is in vitro.

According to other aspects of the disclosure, kits are provided that comprise a container housing any of the compositions disclosed herein.

### BRIEF DESCRIPTION OF DRAWINGS

FIGS. 1A-1E show the identification of a novel non-coding RNA scaffold at the SMN locus, SMN-AS1. FIG. 1A shows mapping of SMN-AS1, as identified by PacBio sequencing, positioned relative to the SMN genes. AS3 and AS4 are northern blot probes. FIG. 1B shows a northern blot detection of human SMN-AS1 at approximately 10 kb in fetal brain and adult lung tissue with AS3 and AS4 probes. Human SMN-AS1 detection in the SMNΔ7 mouse brain (hSMN2+tg) compared to a mouse brain that does not carry the human SMN2 gene. B2M serves as a loading control. FIG. 1C shows that the relative quantitation of SMN-AS1 (light grey bars) correlates with copy number (dark grey bars) as determined by Zhong et al, 2011. GM09677 SMA fibroblasts treated with a SMN-AS1 gapmer ASO showed decreased SMN-AS1 levels. GM20384 cells lacking SMN2, but retaining SMN1, also expressed SMN-AS1. FIG. 1D shows the relative expression levels of SMN-AS1 from 20 human tissue types. The adrenal gland was set to 1 and all other tissues are quantified relative to this. FIG. 1E shows confocal imaging in GM09677 SMA fibroblasts of the nascent SMN pre-mRNA, the mature SMN mRNA and the SMN-AS1 lncRNA in SMA fibroblasts. Signals are offset diagonally (down+right) by 2 pixels.

FIGS. 2A-2H demonstrate that PRC2 is associated with SMN-AS1 and selective dissociation leads to upregulation of SMN expression. FIG. 2A shows a RIP for SUZ12 association with SMN-AS1, SMN-FL mRNA, ANRIL, GAPDH mRNA, and 18S rRNA from SMA fibroblasts with an IgG pulldown (first bar column) or anti-SUZ12 pulldown (second bar column). RIP of RNAs for SMN-AS1, SMN-FL mRNA, ANRIL, GAPDH mRNA, and 18S rRNA were transfected with Oligo 63 (third bar column), or Oligo 52 (fourth bar column). Enrichment is shown as % input (mean+/−S.D; n=4). *P<0.05 by two tailed student's t-test. The sequences, from top to bottom, correspond to SEQ ID NO: 4 (Oligo 63) and SEQ ID NO: 5 (Oligo 52). FIG. 2B shows an EMSA of recombinant human PRC2 containing EZH2, SUZ12, and EED combined with RepA RNA, MBP (441 nt) (SEQ ID NO: 3), SMN-AS1 (PRC2 region) (SEQ ID NO: 1), SMN-AS1 (negative control region) (SEQ ID NO: 2). RNAs bound by PRC2 are the upper bands and unbound RNAs are in the lower band. The fraction of RNA bound by PRC2 is captured in the graph (mean+/−S.D; n=4). The Binding curves are displayed on the bottom (mean+/−S.D; n=3). FIG. 2C shows the RT-qPCR results for SMN-FL, Δ7 SMN, and SMN (exon 1-2) mRNA after SMA fibroblasts were transfected with Oligo 63 for 2 days. Data was normalized to lipid transfection control (mean+/−S.D; n=5). FIG. 2D shows ELISA results for SMN protein after SMA fibroblasts were transfected with Oligo 63 for 5 days (mean+/−S.D; n=3). FIG. 2E shows the Western blot results for SMN and αtubulin after SMA fibroblasts were transfected with Oligo 63 for 5 days (mean+/−S.D; n=2). FIG. 2F shows the RT-qPCR results for human SMN-FL mRNA from mouse cortical neurons gymnotically treated with Oligo 63 at 1.1, 3.3, and 10 μM for 3 or 14 days (mean+/−S.D; n=3). FIG. 2G shows images of untreated human SMA patient iPS-derived motor neuron cultures (left) and motor neuron cultures treated with 10 μM Oligo 63 (right) at day 11. Expression changes of SMN-FL mRNA in human SMA iPS-derived motor neuron cultures after gymnotic treatment with Oligo 63 at 20 μM for 3, 7, 9 or 11 days as a fold change from untreated cells at their respective time points (mean+/−S.D; n=2), measured by RT-qPCR (mean+/−S.D; n=2). FIG. 2H shows RT-qPCR of SMN-FL mRNA in human SMA iPS-derived motor neuron cultures at day 7 after treatment with an EZH2 gapmer ASO (mean+/S.D; n=2).

FIGS. 3A-3G show PRC2 loss and chromatin changes at SMN locus upon Oligo 63 treatment. ChIP at the SMN locus in GM09677 fibroblasts that were untreated, transfected with lipid only, or Oligo 63 for 3 days. ChIP for EZH2 (FIG. 3A), H3K27me3 (FIG. 3B), RNA Polymerase II phosphor-Serine 2 (FIG. 3C), H3K36me3 (FIG. 3D), pan-H3 (FIG. 3E), and H3K4me3 (FIG. 3F) (mean+/−S.D; n=2). FIG. 3G shows the ChIP for HOXC13 promoter from the GM09677 cells under the conditions described above.

FIGS. 4A-4I show that SMN-AS1 recruits PRC2 and SETD2 to modulate SMN mRNA expression. RT-qPCR for SMN-FL mRNA (FIG. 4A), SMN (exon 1-2) mRNA (FIG. 4B), and SMN-AS1 lncRNA (FIG. 4C) from GM09677 fibroblasts transfected with lipid or an SMN-AS1 gapmer for 3 days (mean+/−sd; n=2). FIGS. 4D-4H show ChIP at the SMN locus in GM09677 fibroblasts that were untreated, transfected with lipid only, or SMN-AS 1 gapmer for 3 days. ChIP for EZH2 (FIG. 4D), H3K27me3 (FIG. 4E), H3K36me3 (FIG. 4F), SETD2 (FIG. 4G), and pan H3 (FIG. 4H) (mean+/−S.D; n=2). FIG. 4I shows ChIP for SETD2 at the SMN locus in GM09677 fibroblasts that were untreated, transfected with lipid only, or Oligo 63 for 3 days (mean+/−S.D; n=2).

FIG. 5 depicts EZH2 RIP, illustrating that the enrichment of SMN-AS1 with EZH2 is reduced upon treatment with a steric-blocking oligo, Oligo_63. GM09677 SMA fibroblasts were transfected with steric-blocking oligo (Oligo 63) or an oligo targeting SMN-AS1 but not at the PRC interaction site Oligo 52. The percent input for RNAs that interact with EZH2 and their resultant % input values after Oligo 63 or Oligo 52 treatment is shown. SMN-AS1, ANRIL, GAPDH mRNA, and RPL19 RNAs were assessed.

FIG. 6 shows that SMA fibroblasts transfected with Oligo 63 do not displace SMN-AS1 from the SMN locus. Confocal microscopy detecting the nascent SMN mRNA transcript by probing for SMN intronic sequences (left panel), detecting SMN-AS1 (middle panel), and detecting SMN mRNA exonic sequences (right panel). Outline of the cell (outer outline), the nucleus (middle outline), and the probes (inside outline) show colocalization of SMN-AS1 with the SMN locus.

FIGS. 7A-7C show that the SMN2 locus is a target of PRC2 regulation. FIG. 7A shows ChIP-seq data for EZH2, H3K27me3, and input at the SMN2 locus from GM12878, H1-hESCs, and HepG2 cell lines. The UCSC genome browser data shows mapped reads for EZH2, H3K27me3 and input-associated DNA along the SMN2 locus. The plot is a density graph of signal enrichment with a 25-bp overlap at any given site. FIG. 7B shows RT-qPCR for EZH1 and EZH2 in SMA fibroblast line GM09677 after EZH1 and EZH2 knockdown by transfection of their respective targeting gapmer ASO for 2 days and RT-qPCR for SMN-FL mRNA after EZH1 and EZH2 knockdown (n=2, mean+/−SD). FIG. 7C shows ChIP-qPCR data of EHZ2, H3K27me3, and total H3 from EZH1/EZH2 knockdown compared to the lipid transfection control in the SMA fibroblasts.

FIGS. 8A-8B show the identification of a novel long noncoding RNA at the SMN locus, SMN-AS1. FIG. 8A shows the mapping of SMN-AS1 positioned relative to the SMN genes. The asterisk marks the location of the C-to-T transition found in SMN2. AS3 and AS4 are northern blot probes. FIG. 8B shows anti-SUZ12 nRIP of SMN-AS1 with 2 primer sets (set 1 and set 2), TUG1 RNA, ANRIL RNA, 18S rRNA, GAPDH mRNA, beta-2-microglobulin (B2M), and RPL19 mRNA from SMA fibroblasts with enrichment shown as % input (mean+/−SD; n=3). IgG nRIP served as the negative control for the SUZ12 nRIP.

FIGS. 9A-9I show that PRC2 is associated with SMN-AS1 and that selective dissociation leads to PRC2 loss and chromatin changes at SMN locus. FIG. 9A shows a schematic diagram of the SMN2 locus with ChIP-qPCR primer positions and mixmer ASO positions. FIG. 9B shows RT-qPCR of SMN-FL mRNA after transfection with Oligo 63 and Oligo 52 in SMA fibroblasts for 2 days. FIGS. 9D-9I show ChIP at the SMN2 locus in GM09677 SMA fibroblasts transfected with lipid, or Oligo 63 for (FIG. 9D) EZH2, (FIG. 9E) H3K27me3, (FIG. 9F) RNA Polymerase II phospho-Serine2, (FIG. 9G) H3K36me3, (FIG. 9H) pan-H3, and (FIG. 9I) H3K4me3 (mean+/−S.D; n=2). FIG. 9J shows ChIP for the promoter of HOXC13, a PRC2-regulated gene, for H3, H3K4me3, H3K36me3, RNA Polymerase II, phospho-Serine 2 (RNA PolIIpS2), H3K27me3, and EZH2 after transfection with lipid or Oligo 63 in SMA fibroblasts. (n=2, +/−SEM).

FIGS. 10A-10J show that SMN-AS1 recruits PRC2 and SETD2 to modulate SMN mRNA expression. FIGS. 10A-10C show RT-qPCR for (FIG. 10A) SMN-FL mRNA, (FIG. 10B) SMN (exon 1-2) mRNA, and (FIG. 10C) SMN-AS1 lncRNA from GM09677 SMA fibroblasts (mean+/−S.D; n=2). FIGS. 10D-10H show ChIP at the SMN locus in GM09677 SMA fibroblasts transfected with lipid, or SMN-AS1 gapmer for 3 days (mean+/−S.D; n=2). ChIP for (FIG. 10D) EZH2, (FIG. 10E) H3K27me3, (FIG. 10F) pan-H3, (FIG. 10G) H3K36me3, and (FIG. 10H) SETD2. FIG. 10I depicts the anti-SETD2 nRIP for SMN-AS 1ANRIL, GAPDH mRNA, or RPL19 mRNA with enrichment shown as % input (mean+/−SD; n=2) from GM09677 SMA fibroblasts transfected with lipid (mock) or Oligo 63. IgG nRIP served as the negative control for the SETD2 nRIP from mock or Oligo 63-treated cells. FIG. 10J shows ChIP for SETD2 at the SMN locus in GM09677 SMA fibroblasts transfected with lipid only, or Oligo 63 (mean+/−S.D; n=2).

FIGS. 11A-11B show upregulation of SMN expression upon Oligo 63 treatment. FIG. 11A shows a schematic diagram of the SMN2 locus. FIG. 11B shows RT-qPCR of SMN-FL mRNA in GM09677 fibroblasts that were transfected with 15 nM Oligo 63 or 15 nM SUZ12 gapmer ASO (mean+/−S.D.; n=2). *p<0.05, **p<0.01 using one-way ANOVA. A hexagonally binned scatterplot of the moderated t statistics of the 11,887 annotated genes tested for differential expression post treatment with Oligo 63 or the SUZ12 kd ASO. Each bin is shaded by the number of genes that fall within it, showing the trend of Oligo 63 treated t statistics (and those less significantly differentially expressed genes) generally being reduced compared to their SUZ12 kd ASO counterpart t statistics. SMN is indicated by a black outlined diamond. The Venn diagram shows the significant results (q<0.10) of the pathway analysis utilizing competitive gene set tests on 1,281 canonical pathways after treatment with each oligo. Overlap required that a pathway was both significantly in the same direction. There is significant overlap between the oligo treatments when tested with a hypergeometric test (p=1.36e⁻¹¹), however approximately 4.5 times more pathway gene sets were significantly changing with SUZ12 KD treatment.

FIG. 12 shows the characterization of iPSC line and neuronal cultures representative of a SMA Type 1 patient iPSC line. Panels A-C show positive immunostaining for pluripotency markers, and panel D depicts a normal G-Band karyotype of the iPS cells. Upon neuronal induction and differentiation to the motor neuron cultures, they were found to contain: (panel E) few Nestin progenitors (<10%) and Map2 a/b neurons (dendritic marker), (panel F) pan-neurons marker β3-tubulin (>60%) with few astrolglial (GFAP) cells, and (panel G) mostly SMI32-positive motor neurons (˜40%). Scale bar for panels A-C is 75 μm. Scale bar for panels E-G is 200 μm.

FIGS. 13A-13E show that distinct mechanisms of SMN-FL mRNA generation can be complementary. FIG. 13A shows images of 5025 mouse cortical neurons at day 14 of either mock-treated or with Oligo 92 at 10 μM. FIG. 13B shows RT-qPCR of human SMN-FL mRNA relative to mouse gusb mRNA from the 5025 mouse cortical neurons treated with either 1.1, 3.3 or 10 μM Oligo 92 (mean+/−S.D; n=5). FIG. 13C shows RT-qPCR of human SMN-FL mRNA relative to mouse gusb mRNA from the 5025 mouse cortical neurons treated with 0.1, 0.3, 1.1, 3.3 or 10 μM EZH2 gapmer for 14 days (mean+/−SD; n=2). FIG. 13D shows RT-qPCR results from SMA mouse from 5025WT SMA mouse model cortical neurons treated with a fixed concentration of Oligo 92 (10 μM) in combination with increasing concentrations of a SCO for 14 days (n=2). FIG. 13E shows human SMN protein levels from 5025 SMA mouse cortical neurons treated with a fixed Oligo 92 in combination with increasing concentrations of a SCO for 14 days (n=2), as measured by ELISA.

FIGS. 14A-14J show pathway enrichment in Oligo 63 or SUZ12 kd ASO treated samples. FIG. 14A shows Reactome Double Stranded Break Repair pathway enrichment for Oligo 63. FIG. 14B shows Reactome Double Stranded Break Repair pathway enrichment for SUZ12 kd ASO. FIG. 14C shows Biocarta P53 pathway enrichment for Oligo 63. FIG. 14D shows Biocarta P53 pathway enrichment for SUZ12 kd ASO. FIG. 14E shows Reactome 3′ UTR Mediate Translational Regulation pathway enrichment for Oligo 63. FIG. 14F shows Reactome 3′ UTR Mediate Translational Regulation pathway enrichment for SUZ12 kd ASO. FIG. 14G shows Reactome Cell Cycle Mitotic pathway enrichment for Oligo 63. FIG. 14H shows Reactome Cell Cycle Mitotic pathway enrichment for SUZ12 kd ASO. FIG. 14I shows Reactome G1 S Transition pathway enrichment for Oligo 63. FIG. 14J shows Reactome G1 S Transition pathway enrichment for SUZ12 kd ASO. The values in the bottom panel for FIGS. 14A, 14C, 14E, 14G and 14I are, from left to right, 2e+01, 3e+00, 2e+00, 1e+00, 7e-01, −1e-03, −6e-01, −1e+00, −2e+00, −3e+00 and −1e+01. The values in the bottom panel for FIGS. 14B, 14D, 14F, 14H and 14J are, from left to right, 11.8, 4.6, 3.3., 2.2, 1.2, 0.1, −1.0, −2.3, −3.6, −5.2 and −20.1.

### DETAILED DESCRIPTION OF CERTAIN EMBODIMENTS OF THE DISCLOSURE

It has been found that, while only slightly more than 1% of the human genome is transcribed into mRNAs that encode protein, the majority of the genome is transcribed. The product of much of this transcription is non-coding RNA. Aspects of the disclosure relate to the discovery that certain non-coding RNAs function as scaffolds that recruit activators and repressors to target genes to control their expression. In some embodiments, it has been found that non-coding RNA scaffolds function at the chromosomal locus containing the target gene and modulating expression of the gene through interactions with repressors (e.g., PRC2) and/or activators (e.g., SETD2), for example. Thus, in some embodiments, it has been found that non-coding RNA scaffolds have distinct regions that interact with activators of the target gene and distinct regions that interact with repressors of the target gene. Thus, in some embodiments, non-coding RNA scaffolds can have different effects on gene expression depending on whether they interact with activators or repressors. Aspects of the disclosure relate to the discovery that oligonucleotides complementary with distinct interaction regions of non-coding RNA scaffolds can be used to block interactions between the non-coding RNA scaffolds and either activators or repressors (or both) to alter gene expression. In some embodiments, expression of a target gene can be increased by selectively blocking interaction of repressors (e.g., PRC2) with the non-coding RNA scaffolds. However, in some embodiments, expression of a target gene can be decreased by selectively blocking interaction of activators (e.g., histone modifying enzymes associated with active chromatin, e.g., histone lysine methyltransferases) with the non-coding RNA scaffolds.

In some embodiments, non-coding RNA scaffolds recruit epigenetic regulating complexes that modify chromatin to activate or repress target gene expression. In some embodiments, non-coding RNA scaffolds interact with repressors, such as, for example, Polycomb Repressive Complex 2 (PRC2) and activators, such as, for example, SETD2. Since each non-coding RNA interacts with repressors and activators through distinct sequences it is possible to identify these sites of interaction and efficiently design steric-blocking oligonucleotides that specifically block the binding of repressors or activators to individual non-coding RNAs thus activating or repressing (depending on which site in the non-coding RNA is targeted) expression of a target gene (e.g., a protein coding target gene). Accordingly, using methods provided herein oligonucleotides can induce significant changes in target mRNA and protein levels without affecting neighboring non-target genes.

In some embodiments, a non-coding RNA scaffold has a first interaction region and a second interaction region. In some embodiments, the first interaction region interacts with a repressor. In some embodiments, the second interaction region interacts with an activator. In some embodiments, non-coding RNA scaffolds interact with an activator (e.g., an activating complex). In some embodiments, non-coding RNA scaffolds interact with a repressor (e.g., a repressor complex e.g., PRC2). In some embodiments, a non-coding RNA scaffold may interact with one or more of the following activators: Dlx-2, SETD2, and Trithorax Group Proteins (TrxG). In some embodiments, a non-coding RNA scaffold may interact with one or more of the following repressors: PCG proteins, PRC2 and subunits thereof, EZH2, and G9a.

In some embodiments, the location of an interaction region on the non-coding RNA scaffold can be identified, for example, by an RNA immunoprecipitation assay using an antibody directed against a repressor or activator that interacts with the non-coding RNA scaffold. The interaction region can be identified by sequencing of RNA (or cDNA prepared from the RNA) that immunoprecipitates with the repressor or activator. Regions of the non-coding RNA scaffold that interact with the repressor or activator will generally be protected from nuclease degradation during the immunoprecipitation assay and thus will be detectable in a subsequent sequencing reaction. An electrophoretic mobility shift assay may be used, in some embodiments, to confirm that, or test whether, an interaction region interacts with a particular repressor or activator.

Hybridization techniques, e.g. RNA-FISH, can be utilized to determine whether the ncRNA scaffold of interest localizes to a genomic locus. In some embodiments, standard hybridization techniques, e.g., single-molecule RNA-fluorescent in situ hybridization (RNA-FISH), can be used to determine whether a non-coding RNA scaffold co-localizes with an actively transcribed chromosomal locus or a locus that is not actively transcribed. Immunostaining using antibody that detect the different chromatin states can be combined with the RNA-FISH to identify euchromatin or heterochromatin co-localized with a particular non-coding RNA scaffold.

In some embodiments, oligonucleotides, e.g. steric-blocking oligonucleotides, are designed based on RIP-sequencing (RIP-seq) data to hybridize to sites of interaction (interaction regions) along the ncRNA scaffolds to sterically block interactions with repressors or activators. In some embodiments, the oligonucleotide is a mixmer, comprising locked nucleic acids interspersed with 2′-O-methyl nucleotides, designed to bind its target without inducing degradation of the target. Candidate oligonucleotides, e.g. mixmers, that reduce interaction of a repressor or activator with the non-coding RNA scaffold at the target gene locus. RIP-seq data may be used to identify the first interaction region of the ncRNA scaffold.

As an example of a repressor complex, PRC2 is herein described. In some embodiments, PRC2 acts as a histone methyltransferase and/or epigenetically regulates and/or silences genomic regions (e.g., through methylation of histone H3). Among other functions, PRC2 interacts with long noncoding RNAs (lncRNAs), such as RepA, Xist, and Tsix, to catalyze or facilitate trimethylation of histone H3-lysine27. In some embodiments, PRC2 contains four subunits, Eed, Suz12, RbAp48, and Ezh2. Aspects of the disclosure relate to the recognition that steric-blocking oligonucleotides can be used to prevent or inhibit interaction of PRC2 with a non-coding RNA scaffold (without degrading the non-coding RNA scaffold) to increase expression of an associated target RNA. This is illustrated in the Examples with respect to the SMN gene.

Methods of modulating gene expression are provided, in some embodiments, that may be carried out in vitro, ex vivo, or in vivo. It is understood that any reference to uses of oligonucleotides throughout the description contemplates use of the oligonucleotides in preparation of a pharmaceutical composition or medicament for use in the treatment of condition (e.g., Spinal Muscular Atrophy) associated with altered levels or activity of a target gene. Thus, as one non-limiting example, this aspect of the disclosure includes use of such steric-blocking oligonucleotides in the preparation of a medicament for use in the treatment of disease, in which the treatment involves modulating expression of a target gene.

### Steric Blocking Oligonucleotides for Modulating Expression of Target Genes

In one aspect of the disclosure, steric-blocking oligonucleotides complementary to interaction regions of non-coding RNA scaffolds are provided for modulating expression of target genes in a cell. As used herein, a “steric-blocking oligonucleotide” refers to an oligonucleotide having a structure that sterically hinders binding of an activator or a repressor as described herein to an interaction region of a non-coding RNA scaffold as described herein. In some embodiments, the steric hinderance results in displacement of at least 10%, at least 20%, at least 30%, at least 40%, at least 50%, at least 60%, at least 70%, at least 80%, at least 90% or 100% of the activator or repressor from the non-coding RNA scaffold. Displacement of an activator or repressor (e.g., an activator or repressor that is a protein or protein complex) can be measured, e.g., using chromatin immunoprecipitation (ChIP) or nuclear RNA immunoprecipitation (nRIP) assays as described herein (see, e.g., Examples 1 and 2) on cells treated with a steric-blocking oligonucleotide compared to control cells not treated with a steric-blocking oligonucleotide. Other known methods for measuring displacement include mobility shift assays (see, e.g., Mestre et al. Biochimica et Biophysica Acta 1445 (1999) 86-98). In some embodiments, a steric-blocking oligonucleotide does not induce substantial cleavage or degradation of the non-coding RNA scaffold to which the steric-blocking oligonucleotide binds. In some embodiments, not inducing substantial cleavage or degradation means that the steric-blocking oligonucleotide induces no more than 10%, 5%, 4%, 3%, 2% or 1% cleavage or degradation of the non-coding RNA scaffold in a cell to which the oligonucleotide has been delivered compared to cleavage or degradation of the non-coding RNA scaffold in the cell prior to delivery or to cleavage or degradation of the non-coding RNA scaffold in a control cell to which the oligonucleotide has not been delivered. In some embodiments, a steric-blocking oligonucleotide does not activate RNAse H pathway-mediated degradation of the non-coding RNA scaffold. Non-limiting examples of steric-blocking oligonucleotides include mixmers (e.g., DNA/LNA mixmers, 2′-OME/LNA mixmers) and fully-LNA-modified oligonucleotides. In some embodiments, expression of a target gene is upregulated or increased. However, in some embodiments, expression of a target gene is downregulated or decreased. In some embodiments, steric-blocking oligonucleotides complementary to these interaction regions of non-coding RNA scaffolds inhibits interaction of chromatin modifying complexes that function as activators or repressors with non-coding RNA scaffolds, resulting in chromatin alterations at target genes that are associated with corresponding changes in gene expression. For example, in some embodiments, blocking interaction of chromatin modifying complex that functions as a gene repressor results in reduced methylation of histone H3 and reduced gene inactivation, such that gene expression is upregulated or increased.

An interaction region of a non-coding RNA scaffold refers to a region of the non-coding RNA scaffold that comprises a sequence of nucleotides that interacts directly or indirectly with the repressor (e.g., PRC2 or a subunit thereof) or activator (e.g., SETD2) as described herein. An interaction region can be identified by any method known in the art or described herein.

In some embodiments, a non-coding RNA scaffold has a first interaction region and a second interaction region. In some embodiments, the first interaction region interacts with a repressor. A “repressor” as used herein refers to a molecule (e.g., a protein or protein complex) that decreases expression (either mRNA expression, protein expression, or both) of a target gene in a cell. In some embodiments, a repressor decreases transcription of a gene by inducing epigenetics changes that inhibit or silence the gene. A repressor may decrease expression of a target gene in the cell by at least 5%, 10%, 20%, 30%, 40%, 50%, 60%, 70%, 80%, 90%, or 100% compared to an appropriate control, such as a cell that does not contain the repressor. Exemplary repressors include PCG proteins, PRC2 and subunits thereof, SUZ12, EZH2, EED, AEBP2, JARID2, PCL, RbAp46/48, EZH1 and G9a. In some embodiments, the repressor is EZH2. Methods for measuring expression of mRNA and protein levels are well-known in the art, e.g., quantitative PCR (qPCR), sequencing, Western blot, an enzyme linked immunosorbant assay, mass spectrometry, high-performance liquid chromatography, liquid chromatography, and combinations thereof.

In some embodiments, the second interaction region interacts with an activator. An “activator” as used herein refers to a molecule (e.g., a protein or protein complex) that increases expression (either mRNA expression, protein expression, or both) of a target gene in a cell. In some embodiments, a repressor decreases transcription of a gene by inducing epigenetics changes that induce or activate transcription from the gene. An activator may increase expression of a target gene in the cell by at least 5%, 10%, 20%, 30%, 40%, 50%, 60%, 70%, 80%, 90%, 100%, 150%, 200% or more compared to an appropriate control, such as a cell that does not contain the activator. Exemplary activators include SETD2, Dlx-2, and TrxG. In some embodiments, the activator is SETD2.

In some embodiments, an interaction region is a region of an RNA that crosslinks to a repressor (e.g., a component of PRC2) or an activator (e.g., SETD2) in response to in situ ultraviolet irradiation of a cell that expresses the RNA, or a region of genomic DNA that encodes that RNA region. In some embodiments, an interaction region is a region of an RNA that immunoprecipitates with an antibody that binds specifically to a repressor (e.g., a component of PRC2) or an activator (e.g., SETD2). In some embodiments, an interaction region of a repressor is a region of an RNA that immunoprecipitates with an antibody that binds specifically to SUZ12, EED, EZH2 or RBBP4 (which as noted above are components of PRC2).

In some embodiments, an interaction is a region of an RNA that is protected from nucleases (e.g., RNases) in an RNA-immunoprecipitation assay that employs an antibody that binds specifically to a repressor (e.g., a component of PRC2 such as EZH2) or an activator (e.g., SETD2).

In some embodiments, an interaction region is a region of an RNA within which occurs a relatively high frequency of sequence reads in a sequencing reaction of products of an RNA-immunoprecipitation assay that employs an antibody that binds specifically to a repressor (e.g., a component of PRC2 such as EZH2) or an activator (e.g., SETD2). In some embodiments, an interaction region is a region of an RNA within which occurs a higher frequency (e.g., at least 1.5, 2, or 3 times higher) of sequence reads in a sequencing reaction of products of an RNA-immunoprecipitation assay that employs an antibody that binds specifically to a repressor (e.g., a component of PRC2 such as EZH2) or an activator (e.g., SETD2) compared to the frequency of sequence reads in a control sequencing reaction of products of a control RNA-immunoprecipitation assay that employs a control antibody (e.g., a control isotype-matched antibody). Methods for performing such assays are known in the art (see, e.g., U.S. Patent Application Publication Number US2015/0218560A1, which is herein incorporated by reference in its entirety) and described herein in Examples 1 and 2.

In some embodiments, an interaction region is a sequence of 40 to 60 nucleotides that interacts with a repressor (e.g., a component of PRC2 such as EZH2) or an activator (e.g., SETD2). In some embodiments, an interaction region is a sequence of up to 500 (e.g., up to 500, up to 400, up to 300, up to 200, or up to 100) nucleotides in length that comprises a sequence (e.g., of 40 to 60 nucleotides) that interacts with a repressor (e.g., a component of PRC2) or an activator (e.g., SETD2). In some embodiments, an interaction region has a sequence as set forth in SEQ ID NO: 4 or SEQ ID NO: 1, wherein each T in the sequence is replaced by a U.

In some embodiments, a steric-blocking oligonucleotide is complementary with 5 to 20, 8 to 15, or 8 to 20 nucleotides, e.g., 5, 6, 7, 8, 9, 10, 11, 12, 13, 14, 15, 16, 17, 18, 19, or 20 consecutive nucleotides of an interaction region of a non-coding RNA scaffold. In some embodiments, the region of complementarity is complementary with at least 8, or 5 to 20, 8 to 15, or 8 to 20 consecutive nucleotides of an interaction region of a non-coding RNA scaffold. In some embodiments, a steric-blocking oligonucleotide may have a nucleotide sequence comprising or consisting of 7 to 16, 8 to 15, or 8 to 20, e.g. 7, 8, 9, 10, 11, 12, 13, 14, 15, 16, 17, 18, 19, or 20 contiguous nucleotides complementary with a non-coding RNA scaffold. In some embodiments, the steric-blocking oligonucleotide has a nucleotide sequence complementary with the sequence set forth as:

(SEQ ID NO: 4)
TGTTCCACTATGAAG
or
(SEQ ID NO: 1)
TCCCCTAAACAAAGACGAGGTCTTGCTATGTTGCCCAGACTGGTCTCAAA
CTCCTGAGCTCAAGTGATCCTCCTGCCTCAGCCTTCTAAAATGCCGGGAT
TACAGGCATGAGCCACTGTACCTGGCCTTAAATTTCTTAACATAGCTAGC
ATTTGGAGAAAACCAACCAATAACAACAAAAGACCAACAAAATTAAATTT
AACGAGGACGAAAAGACAGCAAGTGACATAAAAAGTTTAAACATTTTGAT
TTAGACTATGTATCTGTTCCACTATGAAGCTATGAGTAAAAAAAAAAAAT
CAAGCATAAATACTTTCATGCTTTTCCTTAATACACACACACACACACAC
ACACACACAGCTCACATAGCATTTCGAGGGCGATTTTAAGTAAATGTCTT
GGGTAGAACACCTGTTCTAACCCCATCCCAATACACAGTAT.

Oligo 63 described herein is complementary to the bold, underlined sequence.

Complementary, as the term is used in the art, refers to the capacity for precise pairing between two nucleotides. For example, if a nucleotide at a certain position of an oligonucleotide is capable of hydrogen bonding with a nucleotide at the same position of PRC2-associated region, then the oligonucleotide and PRC2-associated region are considered to be complementary to each other at that position. The oligonucleotide and PRC2-associated region are complementary to each other when a sufficient number of corresponding positions in each molecule are occupied by nucleotides that can hydrogen bond with each other through their bases. Thus, “complementary” is a term which is used to indicate a sufficient degree of complementarity or precise pairing such that stable and specific binding occurs between the oligonucleotide, e.g. a steric-blocking oligonucleotide, and target nucleic acid (e.g., a non-coding RNA scaffold, e.g., at a repressor or activator interaction region). For example, if a base at one position of an oligonucleotide is capable of hydrogen bonding with a base at the corresponding position of a PRC2-associated region, then the bases are considered to be complementary to each other at that position. In some embodiments, 100% complementarity is not required.

The steric-blocking oligonucleotide may be at least 80% complementary to (optionally one of at least 85%, 90%, 91%, 92%, 93%, 94%, 95%, 96%, 97%, 98%, 99%, or 100% complementary to) the consecutive nucleotides of a target non-coding RNA scaffold. In some embodiments, the steric-blocking oligonucleotide may contain 1, 2, or 3 base mismatches compared to the portion of the consecutive nucleotides of a target non-coding RNA scaffold. In some embodiments, the steric-blocking oligonucleotide may have up to 3 mismatches over 15 bases, or up to 2 mismatches over 10 bases.

In some embodiments, a complementary nucleotide or oligonucleotide sequence need not be 100% complementary to that of its target to be specifically hybridizable. In some embodiments, a complementary nucleic acid sequence for purposes of the present disclosure is specifically hybridizable when binding of the sequence to the target molecule (e.g., target non-coding RNA scaffold.) interferes with the normal function of the target (e.g., target non-coding RNA scaffold.) to cause a loss of activity (e.g., inhibiting PRC2-associated repression with consequent up-regulation of gene expression) and there is a sufficient degree of complementarity to avoid non-specific binding of the sequence to non-target sequences under conditions in which avoidance of non-specific binding is desired, e.g., under physiological conditions in the case of in vivo assays or therapeutic treatment, and in the case of in vitro assays, under conditions in which the assays are performed under suitable conditions of stringency.

In some embodiments, the steric-blocking oligonucleotide is at least 7 nucleotides in length and up 20 or more nucleotides in length, e.g. 7, 8, 9, 10, 11, 12, 13, 14, 15, 16, 17, 18, 19, 20, or more nucleotides in length.

In some embodiments, any one or more thymidine (T) nucleotides (or modified nucleotide thereof) or uridine (U) nucleotides (or a modified nucleotide thereof) in a sequence provided herein, including a sequence provided in the sequence listing, may be replaced with any other nucleotide suitable for base pairing (e.g., via a Watson-Crick base pair) with an adenosine nucleotide. In some embodiments, any one or more thymidine (T) nucleotides (or modified nucleotide thereof) or uridine (U) nucleotides (or a modified nucleotide thereof) in a sequence provided herein, including a sequence provided in the sequence listing, may be suitably replaced with a different pyrimidine nucleotide or vice versa. In some embodiments, any one or more thymidine (T) nucleotides (or modified nucleotide thereof) in a sequence provided herein, including a sequence provided in the sequence listing, may be suitably replaced with a uridine (U) nucleotide (or a modified nucleotide thereof) or vice versa. In some embodiments, GC content of the steric-blocking oligonucleotide is preferably between about 30-60%. Contiguous runs of three or more Gs or Cs may not be preferable in some embodiments. Accordingly, in some embodiments, the oligonucleotide does not comprise a stretch of three or more guanosine nucleotides.

In some embodiments, the steric-blocking oligonucleotide specifically binds to, or is complementary to an RNA that is encoded in a genome (e.g., a human genome) as a single contiguous transcript (e.g., a non-spliced RNA).

In some embodiments, steric-blocking oligonucleotides disclosed herein may increase expression of mRNA. In some embodiments, expression may be increased by at least about 2 fold, 5 fold, 10 fold, 15 fold, 20 fold, 30 fold, 40 fold, 50 fold or 100 fold, or any range between any of the foregoing numbers. It has also been found that increased mRNA expression has been shown to correlate to increased protein expression.

In some embodiments, steric-blocking oligonucleotides disclosed herein may decrease expression of mRNA. In some embodiments, expression may be decreased by at least about 2 fold, 5 fold, 10 fold, 15 fold, 20 fold, 30 fold, 40 fold, 50 fold or 100 fold, or any range between any of the foregoing numbers. It has also been found that decreased mRNA expression has been shown to correlate to increased protein expression.

Oligonucleotides (e.g. steric-blocking oligonucleotides) that are designed to interact with RNA (e.g., target non-coding RNA scaffold) to modulate gene expression are a distinct subset of base sequences from those that are designed to bind a DNA target (e.g., are complementary to the underlying genomic DNA sequence from which the RNA is transcribed).

### Oligonucleotide Structure and Modifications

The steric-blocking oligonucleotides described herein may be modified, e.g., comprise a modified sugar moiety, a modified internucleoside linkage, a modified nucleotide and/or combinations thereof. In addition, the oligonucleotides may exhibit one or more of the following properties: do not mediate alternative splicing; are not immune stimulatory; are nuclease resistant; have improved cell uptake compared to unmodified oligonucleotides; are not toxic to cells or mammals; or have improved endosomal exit.

Oligonucleotides of the disclosure can be stabilized against nucleolytic degradation such as by the incorporation of a modification, e.g., a nucleotide modification. For example, in some embodiments, nucleic acid sequences of the disclosure include a phosphorothioate at least at the first, second, or third internucleoside linkage at the 5′ or 3′ end of the nucleotide sequence.

As another example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” e.g., comprise nucleic acid analogues in which the ribose ring is “locked” by a methylene bridge connecting the 2′-O atom and the 4′-C atom.

Any of the modified chemistries or formats of oligonucleotides described herein can be combined with each other, and that one, two, three, four, five, or more different types of modifications can be included within the same molecule.

In some embodiments, oligonucleotides include only one type of internucleoside linkage (e.g., oligonucleotides may be fully phosphorothioated). However, in some embodiments, oligonucleotides include a mix of different internucleoside linkages (e.g., a mix of phosphorothioate and phosphodiester linkages). For example, in some embodiments, oligonucleotides may include 50% phosphorothioate linkages and 50% phosphodiester linkages. In some embodiments, oligonucleotides provided herein may have a central stretch of 2, 3, 4, 5, 6, 7, or more nucleotide residues linked by a first linkage type, and flanking nucleotide residues that are linked by a second linkage type. In some embodiments, oligonucleotides provided herein may have a central stretch of 2, 3, 4, 5, 6, 7, or more nucleotide residues linked by phosphodiester linkages, and flanking nucleotide residues that are linked by phosphorothioates. In some embodiments, flanking nucleotide residues are independently 2, 3, 4, 5, 6, 7 or more nucleotide residues in length.

In some embodiments, a steric-blocking oligonucleotide may comprise one or more modified nucleotides (also referred to herein as nucleotide analogs). In some embodiments, the oligonucleotide may comprise at least one ribonucleotide, at least one deoxyribonucleotide, and/or at least one bridged nucleotide. In some embodiments, the oligonucleotide may comprise a bridged nucleotide, such as a locked nucleic acid (LNA) nucleotide, a constrained ethyl (cEt) nucleotide, or an ethylene bridged nucleic acid (ENA) nucleotide. Examples of such nucleotides are disclosed herein. In some embodiments, the oligonucleotide comprises a nucleotide analog disclosed in one of the following United States Patent or Patent Application Publications: U.S. Pat. No. 7,399,845, U.S. Pat. No. 7,741,457, U.S. Pat. No. 8,022,193, U.S. Pat. No. 7,569,686, U.S. Pat. No. 7,335,765, U.S. Pat. No. 7,314,923, U.S. Pat. No. 7,335,765, and U.S. Pat. No. 7,816,333, US 20110009471, the entire contents of each of which are incorporated herein by reference for all purposes. The oligonucleotide may have one or more 2′ O-methyl nucleotides. The oligonucleotide may consist entirely of 2′ O-methyl nucleotides.

Often the steric-blocking oligonucleotide has one or more nucleotide analogues. For example, the oligonucleotide may have at least one nucleotide analogue that results in an increase in Tₘ of the oligonucleotide in a range of 1° C., 2° C., 3° C., 4° C., or 5° C. compared with an oligonucleotide that does not have the at least one nucleotide analogue. The oligonucleotide may have a plurality of nucleotide analogues that results in a total increase in Tₘ of the oligonucleotide in a range of 2° C., 3° C., 4° C., 5° C., 6° C., 7° C., 8° C., 9° C., 10° C., 15° C., 20° C., 25° C., 30° C., 35° C., 40° C., 45° C. or more compared with an oligonucleotide that does not have the nucleotide analogue.

The oligonucleotide may be of up to 50 nucleotides in length in which 2 to 10, 2 to 15, 2 to 16, 2 to 17, 2 to 18, 2 to 19, 2 to 20, 2 to 25, 2 to 30, 2 to 40, 2 to 45, or more nucleotides of the oligonucleotide are nucleotide analogues. The oligonucleotide may be of 7 to 30 nucleotides in length in which 2 to 10, 2 to 15, 2 to 16, 2 to 17, 2 to 18, 2 to 19, 2 to 20, 2 to 25, 2 to 30 nucleotides of the oligonucleotide are nucleotide analogues. The oligonucleotide may be of 7 to 20 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides of the oligonucleotide are nucleotide analogues. Optionally, the oligonucleotides may have every nucleotide except 1, 2, 3, 4, 5, 6, 7, 8, 9, or 10 nucleotides modified.

In some embodiments, the oligonucleotide comprises at least one nucleotide modified at the 2′ position of the sugar, e.g., a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (e.g., higher target binding affinity) than 2′-deoxyoligonucleotides against a given target.

A number of nucleotide modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, modified internucleoside linkages such as phosphorothioates, phosphotriesters, methyl phosphonates, short chain alkyl or cycloalkyl intersugar linkages or short chain heteroatomic or heterocyclic intersugar linkages. In some embodiments, oligonucleotides may have phosphorothioate backbones; heteroatom backbones, such as methylene(methylimino) or MMI backbones; amide backbones (see De Mesmaeker et al. Ace. Chem. Res. 1995, 28:366-374); morpholino backbones (see Summerton and Weller, U.S. Pat. No. 5,034,506); or peptide nucleic acid (PNA) backbones (in which the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity in which the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

Cyclohexenyl nucleic acid oligonucleotide mimetics are described in Wang et al., J. Am. Chem. Soc., 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264, 562; 5, 264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Modified oligonucleotides also include oligonucleotides that are based on or constructed from arabinonucleotide or modified arabinonucleotide residues. Arabinonucleosides are stereoisomers of ribonucleosides, differing only in the configuration at the 2′-position of the sugar ring. In some embodiments, a 2′-arabino modification is 2′-F arabino. In some embodiments, the modified oligonucleotide is 2′-fluoro-D-arabinonucleic acid (FANA) (as described in, for example, Lon et al., Biochem., 41:3457-3467, 2002 and Min et al., Bioorg. Med. Chem. Lett., 12:2651-2654, 2002; the disclosures of which are incorporated herein by reference in their entireties). Similar modifications can also be made at other positions on the sugar, particularly the 3′ position of the sugar on a 3′ terminal nucleoside or in linked oligonucleotides and the 5′ position of 5′ terminal nucleotide.

PCT Publication No. WO 99/67378 discloses arabinonucleic acids (ANA) oligomers and their analogues for improved sequence specific inhibition of gene expression via association to complementary messenger RNA.

Other modifications include ethylene-bridged nucleic acids (ENAs) (e.g., International Patent Publication No. WO 2005/042777, Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Horie et al., Nucleic Acids Symp. Ser (Oxf), 49:171-172, 2005; the disclosures of which are incorporated herein by reference in their entireties). For example, ENAs include, but are not limited to, 2′-O,4′-C-ethylene-bridged nucleic acids.

Examples of LNAs are described in WO/2008/043753 and include compounds of the following general formula.

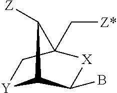

where X and Y are independently selected among the groups —O—,

—S—, —N(H)—, N(R)—, —CH₂— or —CH— (if part of a double bond),

—CH₂—O—, —CH₂—S—, —CH₂—N(H)—, —CH₂—N(R)—, —CH₂—CH₂— or —CH₂—CH— (if part of a double bond),

—CH═CH—, where R is selected from hydrogen and C₁₋₄-alkyl; Z and Z* are independently selected among an internucleoside linkage, a terminal group or a protecting group; B constitutes a natural or non-natural nucleotide base moiety; and the asymmetric groups may be found in either orientation.

In some embodiments, the LNA used in the oligonucleotides described herein comprises at least one LNA unit according any of the formulas

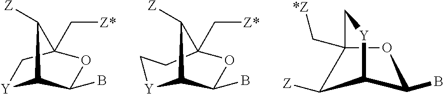

in which Y is —O—, —S—, —NH—, or N(R^{H}); Z and Z* are independently selected among an internucleoside linkage, a terminal group or a protecting group; B constitutes a natural or non-natural nucleotide base moiety, and RH is selected from hydrogen and C₁₋₄-alkyl.

In some embodiments, the Locked Nucleic Acid (LNA) used in the oligonucleotides described herein comprises at least one Locked Nucleic Acid (LNA) unit according any of the formulas shown in Scheme 2 of PCT/DK2006/000512.

In some embodiments, the LNA used in the oligomer of the disclosure comprises internucleoside linkages selected from —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(R^{H})—O—, O—PO(OCH₃)—O—, —O—PO(NR^{H})—O—, —O—PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR^{H}—CO—O—, where R^{H} is selected from hydrogen and C₁₋₄-alkyl.

Examples of LNA units are shown below:

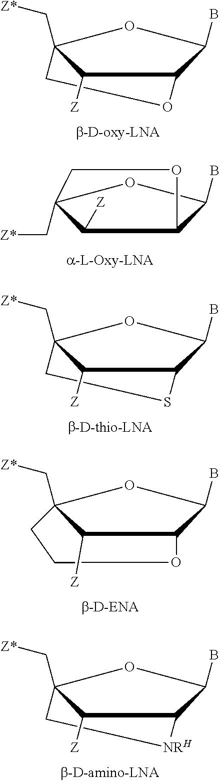

The term “thio-LNA” comprises a locked nucleotide in which at least one of X or Y in the general formula above is selected from S or —CH₂—S—. Thio-LNA can be in both beta-D and alpha-L-configuration.

The term “amino-LNA” comprises a locked nucleotide in which at least one of X or Y in the general formula above is selected from —N(H)—, N(R)—, CH₂—N(H)—, and —CH₂—N(R)— where R is selected from hydrogen and C₁₋₄-alkyl. Amino-LNA can be in both beta-D and alpha-L-configuration.

The term “oxy-LNA” comprises a locked nucleotide in which at least one of X or Y in the general formula above represents —O— or —CH₂—O—. Oxy-LNA can be in both beta-D and alpha-L-configuration.

The term “ena-LNA” comprises a locked nucleotide in which Y in the general formula above is —CH₂—O— (where the oxygen atom of —CH₂—O— is attached to the 2′-position relative to the base B).

One or more substituted sugar moieties can also be included, e.g., one of the following at the 2′ position: OH, SH, SCH₃, F, OCN, OCH₃OCH₃, OCH₃O(CH₂)n CH₃, O(CH₂)n NH₂ or O(CH₂)n CH₃ where n is from 1 to about 10; C1 to C10 lower alkyl, alkoxyalkoxy, substituted lower alkyl, alkaryl or aralkyl; Cl; Br; CN; CF₃; OCF₃; O-, S-, or N-alkyl; O-, S-, or N-alkenyl; SOCH₃; SO₂CH₃; ONO₂; NO₂; N₃; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A modification includes 2′-methoxyethoxy [2′-O—CH₂CH₂OCH₃, also referred as 2′-O-(2-methoxyethyl)] (Martin et al, HeIv. Chim. Acta, 1995, 78, 486). Other modifications include 2′-methoxy (2′-O—CH₃), 2′-propoxy (2′-OCH₂CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Oligonucleotides can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, isocytosine, pseudoisocytosine, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 5-propynyluracil, 8-azaguanine, 7-deazaguanine, N6 (6-aminohexyl)adenine, 6-aminopurine, 2-aminopurine, 2-chloro-6-aminopurine and 2,6-diaminopurine or other diaminopurines. See, e.g., Kornberg, “DNA Replication,” W.H. Freeman & Co., San Francisco, 1980, pp 75-77; and Gebeyehu, G., et al. Nucl. Acids Res., 15:4513 (1987)). A “universal” base, e.g., inosine, can also be included. 5-Me-C substitutions have been shown to increase nucleic acid duplex stability by 0.6-1.2° C. (Sanghvi, in Crooke, and Lebleu, eds., Antisense Research and Applications, CRC Press, Boca Raton, 1993, pp. 276-278) and may be used as base substitutions.

In some embodiments, oligonucleotide modification includes modification of the 5′ or 3′ end of the oligonucleotide. In some embodiments, the 3′ end of the oligonucleotide comprises a hydroxyl group or a thiophosphate. It should be appreciated that additional molecules (e.g. a biotin moiety or a fluorophor) can be conjugated to the 5′ or 3′ end of the oligonucleotide. In some embodiments, the oligonucleotide comprises a biotin moiety conjugated to the 5′ nucleotide.

In some embodiments, an oligonucleotide described herein may be a mixmer or comprise a mixmer sequence pattern. The term ‘mixmer’ refers to oligonucleotides which comprise both naturally and non-naturally occurring nucleotides or comprise two different types of non-naturally occurring nucleotides. Mixmers have a higher binding affinity than unmodified oligonucleotides and may be used to specifically bind a target molecule, e.g., to block a binding site on the target molecule. Generally, mixmers do not recruit an RNAse to the target molecule and thus do not promote cleavage of the target molecule.

In some embodiments, the mixmer comprises or consists of a repeating pattern of nucleotide analogues and naturally occurring nucleotides, or one type of nucleotide analogue and a second type of nucleotide analogue. However, the mixmer need not comprise a repeating pattern and may instead comprise any arrangement of nucleotide analogues and naturally occurring nucleotides or any arrangement of one type of nucleotide analogue and a second type of nucleotide analogue. The repeating pattern, may, for instance be every second or every third nucleotide is a nucleotide analogue, such as LNA, and the remaining nucleotides are naturally occurring nucleotides, such as DNA, or are a 2′ substituted nucleotide analogue such as 2′MOE or 2′ fluoro analogues, or any other nucleotide analogues described herein. It is recognized that the repeating pattern of nucleotide analogues, such as LNA units, may be combined with nucleotide analogues at fixed positions—e.g. at the 5′ or 3′ termini.

In some embodiments, the mixmer does not comprise a region of more than 5, more than 4, more than 3, or more than 2 consecutive naturally occurring nucleotides, such as DNA nucleotides. In some embodiments, the mixmer comprises at least a region consisting of at least two consecutive nucleotide analogues, such as at least two consecutive LNAs. In some embodiments, the mixmer comprises at least a region consisting of at least three consecutive nucleotide analogue units, such as at least three consecutive LNAs.

In some embodiments, the mixmer does not comprise a region of more than 7, more than 6, more than 5, more than 4, more than 3, or more than 2 consecutive nucleotide analogues, such as LNAs. It is to be understood that the LNA units may be replaced with other nucleotide analogues, such as those referred to herein.

In some embodiments, the mixmer comprises at least one nucleotide analogue in one or more of six consecutive nucleotides. The substitution pattern for the nucleotides may be selected from the group consisting of Xxxxxx, xXxxxx, xxXxxx, xxxXxx, xxxxXx and xxxxxX, in which “X” denotes a nucleotide analogue, such as an LNA, and “x” denotes a naturally occurring nucleotide, such as DNA or RNA.

In some embodiments, the mixmer comprises at least two nucleotide analogues in one or more of six consecutive nucleotides. The substitution pattern for the nucleotides may be selected from the group consisting of XXxxxx, XxXxxx, XxxXxx, XxxxXx, XxxxxX, xXXxxx, xXxXxx, xXxxXx, xXxxxX, xxXXxx, xxXxXx, xxXxxX, xxxXXx, xxxXxX and xxxxXX, in which “X” denotes a nucleotide analogue, such as an LNA, and “x” denotes a naturally occurring nucleotide, such as DNA or RNA. In some embodiments, the substitution pattern for the nucleotides may be selected from the group consisting of XxXxxx, XxxXxx, XxxxXx, XxxxxX, xXxXxx, xXxxXx, xXxxxX, xxXxXx, xxXxxX and xxxXxX. In some embodiments, the substitution pattern is selected from the group consisting of xXxXxx, xXxxXx, xXxxxX, xxXxXx, xxXxxX and xxxXxX. In some embodiments, the substitution pattern is selected from the group consisting of xXxXxx, xXxxXx and xxXxXx. In some embodiments, the substitution pattern for the nucleotides is xXxXxx.

In some embodiments, the mixmer comprises at least three nucleotide analogues in one or more of six consecutive nucleotides. The substitution pattern for the nucleotides may be selected from the group consisting of XXXxxx, xXXXxx, xxXXXx, xxxXXX, XXxXxx, XXxxXx, XXxxxX, xXXxXx, xXXxxX, xxXXxX, XxXXxx, XxxXXx, XxxxXX, xXxXXx, xXxxXX, xxXxXX, xXxXxX and XxXxXx, in which “X” denotes a nucleotide analogue, such as an LNA, and “x” denotes a naturally occurring nucleotide, such as DNA or RNA. In some embodiments, the substitution pattern for the nucleotides is selected from the group consisting of XXxXxx, XXxxXx, XXxxxX, xXXxXx, xXXxxX, xxXXxX, XxXXxx, XxxXXx, XxxxXX, xXxXXx, xXxxXX, xxXxXX, xXxXxX and XxXxXx. In some embodiments, the substitution pattern for the nucleotides is selected from the group consisting of xXXxXx, xXXxxX, xxXXxX, xXxXXx, xXxxXX, xxXxXX and xXxXxX. In some embodiments, the substitution pattern for the nucleotides is xXxXxX or XxXxXx. In some embodiments, the substitution pattern for the nucleotides is xXxXxX.

In some embodiments, the mixmer comprises at least four nucleotide analogues in one or more of six consecutive nucleotides. The substitution pattern for the nucleotides may be selected from the group consisting of xXXXX, xXxXXX, xXXxXX, xXXXxX, xXXXXx, XxxXXX, XxXxXX, XxXXxX, XxXXXx, XXxxXX, XXxXxX, XXxXXx, XXXxxX, XXXxXx and XXXXxx, in which “X” denotes a nucleotide analogue, such as an LNA, and “x” denotes a naturally occurring nucleotide, such as DNA or RNA.

In some embodiments, the mixmer comprises at least five nucleotide analogues in one or more of six consecutive nucleotides. The substitution pattern for the nucleotides may be selected from the group consisting of xXXXXX, XxXXXX, XXxXXX, XXXxXX, XXXXxX and XXXXXx, in which “X” denotes a nucleotide analogue, such as an LNA, and “x” denotes a naturally occurring nucleotide, such as DNA or RNA.

The oligonucleotide may comprise a nucleotide sequence having one or more of the following modification patterns.

(a) (X)Xxxxxx, (X)xXxxxx, (X)xxXxxx, (X)xxxXxx, (X)xxxxXx and (X)xxxxxX,

(b) (X)XXxxxx, (X)XxXxxx, (X)XxxXxx, (X)XxxxXx, (X)XxxxxX, (X)xXXxxx, (X)xXxXxx, (X)xXxxXx, (X)xXxxxX, (X)xxXXxx, (X)xxXxXx, (X)xxXxxX, (X)xxxXXx, (X)xxxXxX and (X)xxxxXX,

(c) (X)XXXxxx, (X)xXXXxx, (X)xxXXXx, (X)xxxXXX, (X)XXxXxx, (X)XXxxXx, (X)XXxxxX, (X)xXXxXx, (X)xXXxxX, (X)xxXXxX, (X)XxXXxx, (X)XxxXXx (X)XxxxXX, (X)xXxXXx, (X)xXxxXX, (X)xxXxXX, (X)xXxXxX and (X)XxXxXx,

(d) (X)xxXXX, (X)xXxXXX, (X)xXXxXX, (X)xXXXxX, (X)xXXXXx, (X)XxxXXXX, (X)XxXxXX, (X)XxXXxX, (X)XxXXx, (X)XXxxXX, (X)XXxXxX, (X)XXxXXx, (X)XXXxxX, (X)XXXxXx, and (X)XXXXxx,

(e) (X)xXXXXX, (X)XxXXXX, (X)XXxXXX, (X)XXXxXX, (X)XXXXxX and (X)XXXXXx, and

(f) XXXXXX, XxXXXXX, XXxXXXX, XXXxXXX, XXXXxXX, XXXXXxX and XXXXXXx, in which “X” denotes a nucleotide analogue, (X) denotes an optional nucleotide analogue, and “x” denotes a DNA or RNA nucleotide unit. Each of the above listed patterns may appear one or more times within an oligonucleotide, alone or in combination with any of the other disclosed modification patterns.

In some embodiments, the mixmer contains a modified nucleotide, e.g., an LNA, at the 5′ end. In some embodiments, the mixmer contains a modified nucleotide, e.g., an LNA, at the first two positions, counting from the 5′ end.

In some embodiments, the mixmer is incapable of recruiting RNAseH. Oligonucleotides that are incapable of recruiting RNAseH are described, for example, in WO2007/112754 and WO2007/112753. Mixmers may be designed to comprise a mixture of affinity enhancing nucleotide analogues, such as in non-limiting example LNA nucleotides and 2′-O-methyl nucleotides. In some embodiments, the mixmer comprises modified internucleoside linkages (e.g., phosphorothioate internucleoside linkages or other linkages) between at least two, at least three, at least four, at least five or more nucleotides.

A mixmer may be produced using any appropriate method. Representative U.S. patents and U.S. patent publications that report the preparation of mixmers include U.S. patent publication Nos. US20060128646, US20090209748, US20090298916, US20110077288, and US20120322851, and U.S. Pat. No. 7,687,617.

In some embodiments, the oligonucleotides are chemically linked to one or more moieties or conjugates that enhance the activity, cellular distribution, or cellular uptake of the oligonucleotide. For example, one or more oligonucleotides, of the same or different types, can be conjugated to each other; or oligonucleotides can be conjugated to targeting moieties with enhanced specificity for a cell type or tissue type. Such moieties include, but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552, 538; 5,578,717, 5,580,731; 5,580,731; 5,591,584; 5,109,124; 5,118,802; 5,138,045; 5,414,077; 5,486, 603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

These moieties or conjugates can include conjugate groups covalently bound to functional groups such as primary or secondary hydroxyl groups. Conjugate groups of the disclosure include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this disclosure, include groups that improve uptake, enhance resistance to degradation, and/or strengthen sequence-specific hybridization with the target nucleic acid. Groups that enhance the pharmacokinetic properties, in the context of this disclosure, include groups that improve uptake, distribution, metabolism or excretion of the compounds of the present disclosure. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

### Methods for Modulating Gene Expression

In some embodiments, methods are provided for increasing expression of a target gene and its translated protein in a cell. The methods, in some embodiments, involve delivering to the cell a steric-blocking oligonucleotide complementary with a first interaction region of a target non-coding RNA scaffold and a steric-blocking oligonucleotide complementary with a second interaction region of a target non-coding RNA scaffold, in amounts sufficient to increase expression of a mature mRNA of the target gene in the cell. In some embodiments, multiple different oligonucleotides may be delivered together or separately. In such embodiments, the oligonucleotides may be linked together or unlinked.

In some embodiments, methods are provided for treating conditions associated with altered gene expression relating to non-coding RNA scaffolds (e.g., spinal muscular atrophy or other conditions (e.g., ALS)) in a subject. The methods, in some embodiments, involve administering to a subject a steric-blocking oligonucleotide complementary with an interaction region of a target non-coding RNA scaffold that interacts with a repressor or activator, in amounts sufficient to modulate expression of protein translated from a target gene in the subject to levels sufficient to improve one or more conditions associated with a disease of the subject.

In one aspect, the disclosure relates to methods for modulating target gene expression in a cell for research purposes (e.g., to study the function of the gene in the cell). In another aspect, the disclosure relates to methods for modulating gene expression in a cell for gene or epigenetic therapy. The cells can be in vitro, ex vivo, or in vivo (e.g., in a subject who has a disease resulting from reduced expression or activity of a target gene or protein, respectively). In some embodiments, methods for modulating gene expression in a cell comprise delivering a steric-blocking oligonucleotide as described herein. In some embodiments, delivery of the steric-blocking oligonucleotide to the cell results in expression of gene that is at least 5%, 10%, 20%, 30%, 40%, 50%, 60%, 70%, 80%, 90%, 100%, 200% or more greater than expression of gene in a control cell to which the steric-blocking oligonucleotide has not been delivered. In certain embodiments, delivery of the steric-blocking oligonucleotide to the cell results in of expression of gene that is at least 50% greater than of expression of gene in a control cell to which the steric-blocking oligonucleotide has not been delivered.

In another aspect of the disclosure, methods comprise administering to a subject (e.g. a human) a composition comprising a steric-blocking oligonucleotide as described herein to increase protein levels in the subject. In some embodiments, the increase in protein levels is at least 5%, 10%, 20%, 30%, 40%, 50%, 60%, 70%, 80%, 90%, 100%, 200%, or more, higher than the protein levels in the subject before administering.

As another example, to increase expression of a target gene in a cell, the methods include introducing into the cell a steric-blocking oligonucleotide that is sufficiently complementary to a target non-coding RNA scaffold encoded from a genomic position encompassing or in proximity to the target gene.

In another aspect of the disclosure provides methods of treating a condition (e.g., Spinal Muscular Atrophy) associated with decreased expression of a target gene in a subject, the method comprising administering a steric-blocking oligonucleotide as described herein that blocks interaction of a non-coding RNA scaffold with a repressor. In another aspect of the disclosure provides methods of treating a condition associated with increased expression of a target gene in a subject, the method comprising administering a steric-blocking oligonucleotide as described herein that blocks interaction of a non-coding RNA scaffold with an activator.

A subject can include a non-human mammal, e.g. mouse, rat, guinea pig, rabbit, cat, dog, goat, cow, or horse. In preferred embodiments, a subject is a human. Steric blocking oligonucleotides have been employed as therapeutic moieties in the treatment of disease states in animals, including humans. Steric blocking oligonucleotides can be useful therapeutic modalities that can be configured to be useful in treatment regimens for the treatment of cells, tissues, and animals, especially humans.

For therapeutics, an animal, preferably a human, suspected of having a particular condition is treated by administering steric-blocking oligonucleotide in accordance with this disclosure. For example, in one non-limiting embodiment, the methods comprise the step of administering to the animal in need of treatment, a therapeutically effective amount of a steric-blocking oligonucleotide as described herein.

### Formulation, Delivery, And Dosing

The oligonucleotides described herein can be formulated for administration to a subject for treating a condition (e.g., Spinal Muscular Atrophy) associated with aberrant levels of protein translated from a target gene (e.g. SMN). It should be understood that the formulations, compositions and methods can be practiced with any of the oligonucleotides disclosed herein. In some embodiments, formulations are provided that comprise a steric-blocking oligonucleotide complementary with first interaction region of a target non-coding RNA scaffold and/or a steric-blocking oligonucleotide complementary with a second interaction region of a target non-coding RNA scaffold. In some embodiments, formulations are provided that comprise a steric-blocking oligonucleotide complementary to a first interaction region of a non-coding RNA scaffold that is linked via a linker with a steric-blocking oligonucleotide complementary to a second interaction region of a non-coding RNA scaffold. Thus, it should be appreciated that in some embodiments, the steric-blocking oligonucleotides are linked, and in other embodiments, the oligonucleotides are not linked. Oligonucleotides that are not linked may be administered to a subject or delivered to a cell simultaneously (e.g., within the same composition) or separately.

The formulations may conveniently be presented in unit dosage form and may be prepared by any appropriate method. The amount of active ingredient (e.g., an oligonucleotide or compound of the disclosure) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect, e.g. tumor or symptom regression.

Pharmaceutical formulations of this disclosure can be prepared according to any appropriate method for the manufacture of pharmaceuticals. Such formulations can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

A formulated oligonucleotide composition can assume a variety of states. In some examples, the composition is at least partially crystalline, uniformly crystalline, and/or anhydrous (e.g., less than 80, 50, 30, 20, or 10% water). In another example, the oligonucleotide is in an aqueous phase, e.g., in a solution that includes water. The aqueous phase or the crystalline compositions can, e.g., be incorporated into a delivery vehicle, e.g., a liposome (particularly for the aqueous phase) or a particle (e.g., a microparticle as can be appropriate for a crystalline composition). Generally, the oligonucleotide composition is formulated in a manner that is compatible with the intended method of administration.

In some embodiments, the composition is prepared by at least one of the following methods: spray drying, lyophilization, vacuum drying, evaporation, fluid bed drying, or a combination of these techniques; or sonication with a lipid, freeze-drying, condensation, and other self-assembly.

Oligonucleotide preparations can be formulated or administered (together or separately) in combination with another agent, e.g., another therapeutic agent or an agent that stabilizes a oligonucleotides, e.g., a protein that complexes with oligonucleotides. Still other agents include chelators, e.g., EDTA (e.g., to remove divalent cations such as Mg²⁺), salts, RNAsc inhibitors (e.g., a broad specificity RNAsc inhibitor such as RNAsin) and so forth. In some embodiments, the other agent used in combination with the oligonucleotide is an agent that also regulates gene expression. In some embodiments, the other agent is a growth hormone, a histone deacetylase inhibitor, a hydroxycarbamide (hydroxyurea), a natural polyphenol compound (e.g., resveratrol, curcumin), prolactin, or salbutamol. Examples of histone deacetylase inhibitors that may be used include aliphatic compounds (e.g., butyrates (e.g., sodium butyrate and sodium phenylbutyrate) and valproic acid), benzamides (e.g., M344), and hydroxamic acids (e.g., CBHA, SBHA, Entinostat (MS-275)) Panobinostat (LBH-589), Trichostatin A, Vorinostat (SAHA)),

In some embodiments, the oligonucleotide preparation includes another oligonucleotide, e.g., a second oligonucleotide that modulates expression and/or mRNA processing of a second gene or a second oligonucleotide that modulates expression of the first gene. Still other preparations can include at least 3, 5, ten, twenty, fifty, or a hundred or more different oligonucleotide species. Such oligonucleotides can mediate gene expression with respect to a similar number of different genes. In one embodiment, the oligonucleotide preparation includes at least a second therapeutic agent (e.g., an agent other than an oligonucleotide).

### Route of Delivery

A composition that includes oligonucleotides can be delivered to a subject by a variety of routes. Exemplary routes include: intrathecal, intracerebral, intramuscular, intravenous, intradermal, topical, rectal, parenteral, anal, intravaginal, intranasal, pulmonary, ocular, etc. The term “therapeutically effective amount” is the amount of oligonucleotide present in the composition that is needed to provide the desired level of gene expression in the subject to be treated to give the anticipated physiological response. The term “physiologically effective amount” is that amount delivered to a subject to give the desired palliative or curative effect. The term “pharmaceutically acceptable carrier” means that the carrier can be administered to a subject with no significant adverse toxicological effects to the subject.

The oligonucleotide molecules of the disclosure can be incorporated into pharmaceutical compositions suitable for administration. Such compositions typically include one or more species of oligonucleotides and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration.

The pharmaceutical compositions of the present disclosure may be administered in a number of ways depending upon whether local or systemic treatment is desired and upon the area to be treated. Administration may be topical (including ophthalmic, vaginal, rectal, intranasal, transdermal), oral, or parenteral. In some embodiments, administration is parenteral, e.g. intramuscular, intravenous (e.g., as a bolus or as a diffusible infusion), intradermal, intraperitoneal, intrathecal, intraventricular, intracranial, subcutaneous, transmucosal, buccal, sublingual, endoscopic, rectal, oral, vaginal, topical, pulmonary, intranasal, urethral, or ocular. Administration can be provided by the subject or by another person, e.g., a health care provider.

The route and site of administration may be chosen to enhance targeting. For example, to target muscle cells, intramuscular injection into the muscles of interest would be a logical choice.

Topical administration refers to the delivery to a subject by contacting the formulation directly to a surface of the subject. The most common form of topical delivery is to the skin, but a composition disclosed herein can also be directly applied to other surfaces of the body, e.g., to the eye, a mucous membrane, to surfaces of a body cavity or to an internal surface Formulations for topical administration may include transdermal patches, ointments, lotions, creams, gels, drops, suppositories, sprays, liquids, and powders. Conventional pharmaceutical carriers, aqueous, powder or oily bases, thickeners and the like may be necessary or desirable.

Compositions for oral administration include powders or granules, suspensions or solutions in water, syrups, slurries, emulsions, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening and/or flavoring agents can be added.

Parenteral administration includes intravenous drip, subcutaneous, intraperitoneal or intramuscular injection, intrathecal or intraventricular administration. In some embodiments, parental administration involves administration directly to the site of disease (e.g. injection into a tumor).

Formulations for parenteral administration may include sterile aqueous solutions which may also contain buffers, diluents, and other suitable additives. Intraventricular injection may be facilitated by an intraventricular catheter, for example, attached to a reservoir. For intravenous use, the total concentration of solutes should be controlled to render the preparation isotonic.

Pulmonary delivery compositions can be delivered by inhalation by the patient of a dispersion so that the composition, preferably oligonucleotides, within the dispersion can reach the lung where it can be readily absorbed through the alveolar region directly into blood circulation. Pulmonary delivery can be effective both for systemic delivery and for localized delivery to treat diseases of the lungs.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Suitable pH adjusters or buffers include organic salts prepared from organic acids and bases, such as sodium citrate, sodium ascorbate, and the like; sodium citrate is preferred. Pulmonary administration of a micellar oligonucleotide formulation may be achieved through metered dose spray devices with propellants such as tetrafluoroethane, heptafluoroethane, dimethylfluoropropane, tetrafluoropropane, butane, isobutane, dimethyl ether, and other non-CFC and CFC propellants.

Exemplary delivery devices include devices which are introduced into the vasculature, e.g., devices inserted into the lumen of a vascular tissue, or which devices themselves form a part of the vasculature, including stents, catheters, heart valves, and other vascular devices. These devices, e.g., catheters or stents, can be placed in the vasculature of the lung, heart, or leg.

Other devices include non-vascular devices, e.g., devices implanted in the peritoneum, or in organ or glandular tissue, e.g., artificial organs. The device can release a therapeutic substance in addition to an oligonucleotide, e.g., a device can release insulin.

In some embodiments, unit doses or measured doses of a composition that includes oligonucleotide are dispensed by an implanted device. The device can include a sensor that monitors a parameter within a subject. For example, the device can include pump, e.g., and, optionally, associated electronics.

Tissue, e.g., cells or organs can be treated with oligonucleotides, ex vivo and then administered or implanted in a subject. The tissue can be autologous, allogeneic, or xenogeneic tissue. For example, tissue can be treated to reduce graft v. host disease. In some embodiments, the tissue is allogeneic and the tissue is treated to treat a disorder characterized by unwanted gene expression in that tissue. For example, tissue, e.g., hematopoietic cells, e.g., hone marrow hematopoietic cells, can be treated to inhibit unwanted cell proliferation. Introduction of treated tissue, whether autologous or transplant, can be combined with other therapies. In some implementations, the oligonucleotide treated cells are insulated from other cells, e.g., by a semi-permeable porous barrier that prevents the cells from leaving the implant, but enables molecules from the body to reach the cells and molecules produced by the cells to enter the body. In one embodiment, the porous barrier is formed from alginate.

### Dosage

In some aspects, the disclosure features methods of administering oligonucleotides (e.g., as a compound or as a component of a composition) to a subject (e.g., a human subject). For example, transcriptional oligonucleotides can be effective in vivo when combined with either oligonucleotides or small molecules that promote correct splicing of target gene transcripts. A variety of doses, routes of administration, and dosing regiments can be employed.

In some embodiments, in order to access the central nervous system (CNS), the two agents may be administered by either intracerebroventricular (ICV) or intrathecal (IT) injection. In human clinical use, IT injection is a useful route of administration into the CNS. The IT injection can be a bolus injection or longer term infusion. Systemic exposure administration of oligonucleotides may be achieved by subcutaneous (SC) injection, although intravenous (IV) and intraperitoneal (IP) routes also may be used. To achieve both CNS and peripheral tissue exposure in human patients, both IT and SC injections may be used. Due to the long half-life of oligonucleotides in the brain and spinal cord, IT injections may be in a range of once every 3 months to once every 6 months; however, in some embodiments, multiple injections at closer intervals may be used at the start of treatment as a “loading” regimen. A variety of dose schedules may be used for SC injection, with once monthly injection being an example regimen.

In some embodiments, the methods involve administering an agent (e.g., a oligonucleotide,) in a unit dose to a subject. In one embodiment, the unit dose is between about 10 mg and 25 mg per kg of bodyweight. In one embodiment, the unit dose is between about 1 mg and 100 mg per kg of bodyweight. In one embodiment, the unit dose is between about 0.1 mg and 500 mg per kg of bodyweight. In some embodiments, the unit dose is more than 0.001, 0.005, 0.01, 0.05, 0.1, 0.5, 1, 2, 5, 10, 25, 50, or 100 mg per kg of bodyweight.

The defined amount can be an amount effective to treat or prevent a disease or disorder, e.g., a disease or disorder associated with the target gene. The unit dose, for example, can be administered by injection (e.g., intravenous or intramuscular), an inhaled dose, or a topical application.

In some embodiments, the unit dose is administered daily. In some embodiments, less frequently than once a day, e.g., less than every 2, 4, 8, or 30 days. In another embodiment, the unit dose is not administered with a frequency (e.g., not a regular frequency). For example, the unit dose may be administered a single time. In some embodiments, the unit dose is administered more than once a day, e.g., once an hour, every two hours, every four hours, every eight hours, every twelve hours, etc.

In one embodiment, a subject is administered an initial dose and one or more maintenance doses of a oligonucleotide. The maintenance dose or doses are generally lower than the initial dose, e.g., one-half less of the initial dose. A maintenance regimen can include treating the subject with a dose or doses ranging from 0.0001 to 100 mg/kg of body weight per day, e.g., 100, 10, 1, 0.1, 0.01, 0.001, or 0.0001 mg per kg of bodyweight per day. The maintenance doses may be administered no more than once every 1, 5, 10, or 30 days. Further, the treatment regimen may last for a period of time which will vary depending upon the nature of the particular disease, its severity, and the overall condition of the patient. In some embodiments, the dosage may be delivered no more than once per day, e.g., no more than once per 24, 36, 48, or more hours, e.g., no more than once for every 5 or 8 days. Following treatment, the patient can be monitored for changes in the subject's condition and for alleviation of the symptoms of the disease state. The dosage of the oligonucleotide may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances.

The steric-blocking oligonucleotides may be administered together, e.g., simultaneously. Alternatively, the dosing of the two oligonucleotides could be staggered such that one may be administered prior to the other. If simultaneous administration is desired, two oligonucleotides (e.g., targeting different non-coding RNA scaffold associated with different genes or targeting different regions within a single non-coding RNA scaffold) either could be mixed together or actually covalently linked in one chemical composition. For instance, two oligonucleotides could be linked in a Multi-Target Oligonucleotide (MTO). In this composition, the two separate oligonucleotides sequences are joined together in one oligonucleotide and are separated by a cleavable linker. This linker could be a nucleotide or non-nucleotide linker. In one embodiment, the two oligonucleotides sequences are separated by 2, 3 or 4 DNA nucleotides, typically poly dA or dT. In some embodiments, the target gene upregulating sequences can be heavily modified for increased stability. The MTO is stable in blood and tissues. Once taken up into cells, the linker in the MTO is cleaved within endosomes in the cells, thus releasing the two separate gene targeting oligonucleotides to act via their distinct mechanisms of action and target sites.

Accordingly, in some embodiments, a pharmaceutical composition includes a plurality of oligonucleotide species. In some embodiments, the pharmaceutical composition comprises a first oligonucleotide complementary with a first interaction region of a non-coding RNA scaffold, and a second oligonucleotide complementary to a second interaction region of a non-coding RNA scaffold. In some embodiments, the pharmaceutical composition includes a compound comprising the general formula A-B-C, in which A is a steric-blocking oligonucleotide complementary to a first interaction region of a non-coding RNA scaffold, B is a linker, and C is a steric-blocking oligonucleotide complementary to a second interaction region of a non-coding RNA scaffold.

Following successful treatment, it may be desirable to have the patient undergo maintenance therapy to prevent the recurrence of the disease state, in which the compound of the disclosure is administered in maintenance doses, ranging from 0.0001 mg to 100 mg per kg of body weight.

The concentration of the oligonucleotide composition is an amount sufficient to be effective in treating or preventing a disorder or to regulate a physiological condition in humans. The concentration or amount of oligonucleotide administered will depend on the parameters determined for the agent and the method of administration, e.g. intramuscular.

Certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a oligonucleotide can include a single treatment or, preferably, can include a series of treatments. It will also be appreciated that the effective dosage of a oligonucleotide used for treatment may increase or decrease over the course of a particular treatment. For example, the subject can be monitored after administering a oligonucleotide composition. Based on information from the monitoring, an additional amount of the oligonucleotide composition can be administered.

### Kits

In certain aspects of the disclosure, kits are provided, comprising a container housing a composition comprising a steric-blocking oligonucleotide. In some embodiments, the composition is a pharmaceutical composition comprising a steric-blocking oligonucleotide and a pharmaceutically acceptable carrier. In some embodiments, the individual components of the pharmaceutical composition may be provided in one container. Alternatively, it may be desirable to provide the components of the pharmaceutical composition separately in two or more containers, e.g., one container for steric-blocking oligonucleotides, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

The present disclosure is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Example 1

Spinal muscular atrophy (SMA) is a group of hereditary diseases that causes muscle damage leading to impaired muscle function, difficulty breathing, frequent respiratory infection, and eventually death. SMA is the leading genetic cause of death in infants and children. There are four types of SMA that are classified based on the onset and severity of the disease. SMA type 1 is the most severe form and is one of the most common causes of infant mortality, with symptoms of muscle weakness and difficulty breathing occurring at birth. SMA type II occurs later, with muscle weakness and other symptoms developing from ages 6 month to 2 years. Symptoms appear in SMA type III during childhood and in SMA type IV, the mildest form, during adulthood. All four types of SMA have been found to be associated with mutations in the Survival of Motor Neuron (SMN) gene family, particularly SMN1.

SMN protein plays a critical role in RNA splicing in motor neurons. Loss of function of the SMN1 gene is responsible for SMA. Humans have an extra SMN gene copy, called SMN2. Both SMN genes reside within a segmental duplication on Chromosome 5q13 as inverted repeats. SMN1 and SMN2 are almost identical. In some cases, SMN1 and SMN2 differ by 11 nucleotide substitutions, including seven in intron 6, two in intron 7, one in coding exon 7, and one in non-coding exon 8. The substitution in exon 7 involves a translationally silent C to T transition compared with SMN1, that results in alternative splicing because the substitution disrupts recognition of the upstream 3′ splice site, in which exon 7 is frequently skipped during precursor mRNA splicing. This mutation causes the inefficient splicing of SMN2 transcripts.

While most SMN1 transcripts are spliced properly, leading to the translation of a full-length protein, the majority of SMN2 transcripts lack exon 7. Consequently, SMN2 encodes primarily the exon 7-skipped protein isoform (“dc17,” SMNΔ7), which is truncated protein which is unstable, mislocalized, partially functional, and rapidly degraded in cells. Therefore, the SMN2 locus leads to the expression of far less SMN protein than the SMN1 gene. SMA patients have mutations in the SMN1 gene and rely solely on the SMN2 gene for SMN protein production. It is apparent that the SMN2 gene does produce some functional SMN protein since patients lacking SMN1 but having increased DNA copy number of the SMN2 gene have a more mild disease phenotype. In addition to SMA, altered SMN expression has been implicated in other motor neuron diseases, such as Amyotrophic Lateral Sclerosis (ALS), Primary Lateral Sclerosis, Progressive Muscular Atrophy, Progressive Bulbar Palsy or Pseudobulbar Palsy.

Methods and a related steric-blocking oligonucleotide that elevates SMN protein levels in cells (e.g., cells of a SMA patient), e.g., by increasing SMN2 transcription and correcting its splicing, are provided herein. Further aspects are described in detailed herein.

Modulation of SMN Expression by Targeting lncRNA Recruitment by Chromatin Regulators

Spinal motor neurons are the most sensitive to the genetic SMN defect and motor function loss putatively caused by their early death is the primary symptom in SMA patients. Below, it is shown that SMN2 can produce full-length SMN protein in SMA patient cells that is equivalent to that seen in unaffected cells by selectively and sterically blocking a transcriptional repressive complex from being recruited to the SMN genes by a novel long noncoding RNA. The long noncoding RNA, SMN-AS1 acts in cis to recruit Polycomb Repressive Complex 2 (PRC2) to SMN2. By using a short steric-blocking (SB) oligonucleotide that binds to SMN-AS1 at the site of the PRC2 interaction, PRC2 association and H3K27me3 levels are reduced within the SMN2 gene. As a result, increased SMN mRNA and protein in SMA patient fibroblasts and neuronal cultures were observed. Using this technology to sterically block PRC2 and lncRNA interactions, it is possible to selectively block PRC2 activity and effectively upregulate target gene expression for therapeutic benefit.

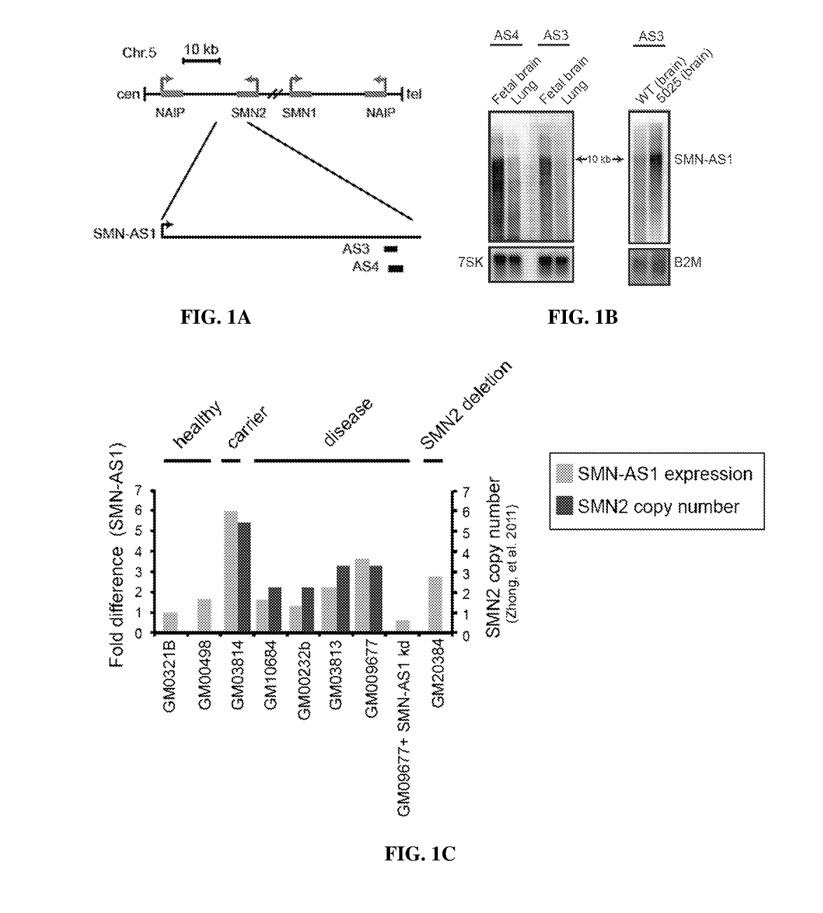

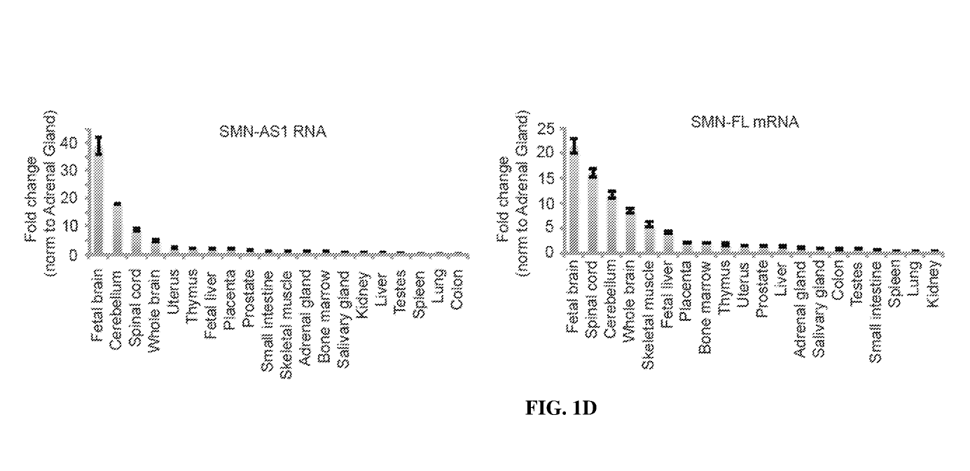

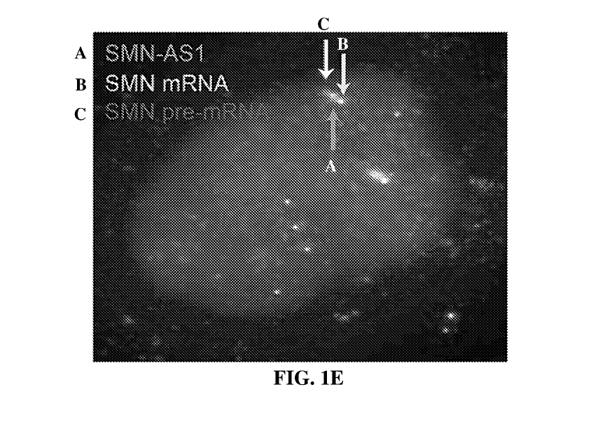

The SMN-AS1 is transcribed antisense to the SMN1 and SMN2 genes. Northern blot, RNA sequencing, and PacBio sequencing data confirmed that this lncRNA is approximately 10 kb, unspliced, and not polyadenylated (FIGS. 1A-1B). The AS3 probe detects SMN-AS1 in the SMNΔ7 mouse model carrying the human SMN2 transgene, but not in wild-type mice carrying only the mouse Smn gene. Strand-specific RT-qPCR shows that SMN-AS1 levels correlate with SMN2 copy number, as determined using droplet dPCR by Zhong et al. (3) (FIG. 1C). Furthermore, a gapmer targeting SMN-AS1 is effective at knocking down SMN-AS1 in the GM09677 fibroblasts. This lncRNA is ubiquitously expressed; however, its expression is higher in CNS tissue, e.g. the brain and spinal cord, relative to the periphery (FIG. 1B, 1D). Furthermore, there is a direct correlation between SMN-FL expression and the amount of SMN-AS1 detected in selected tissues. Single-molecule RNA-fluorescent in situ hybridization (RNA-FISH) demonstrated that SMN-AS1 co-localizes with the actively transcribed SMN locus, whose expression is predominantly specific to one allele (FIG. 1E). Nascent SMN transcripts, detected by RNA-FISH probes designed to hybridize to the introns, colocalize with SMN-AS1 lncRNA. This suggests that SMN-AS1 localizes to the SMN genomic locus and may function in cis.

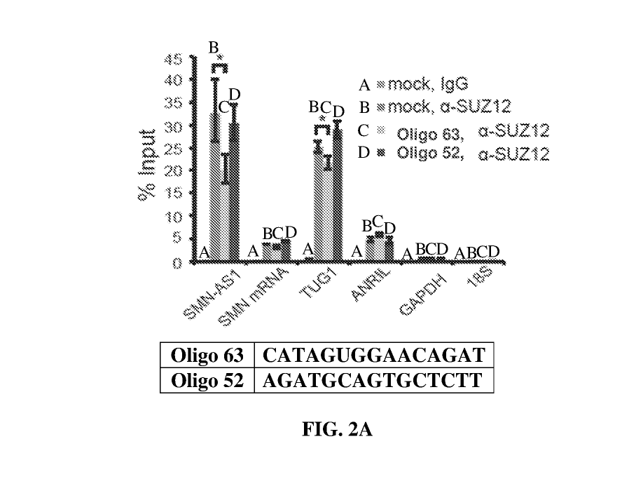

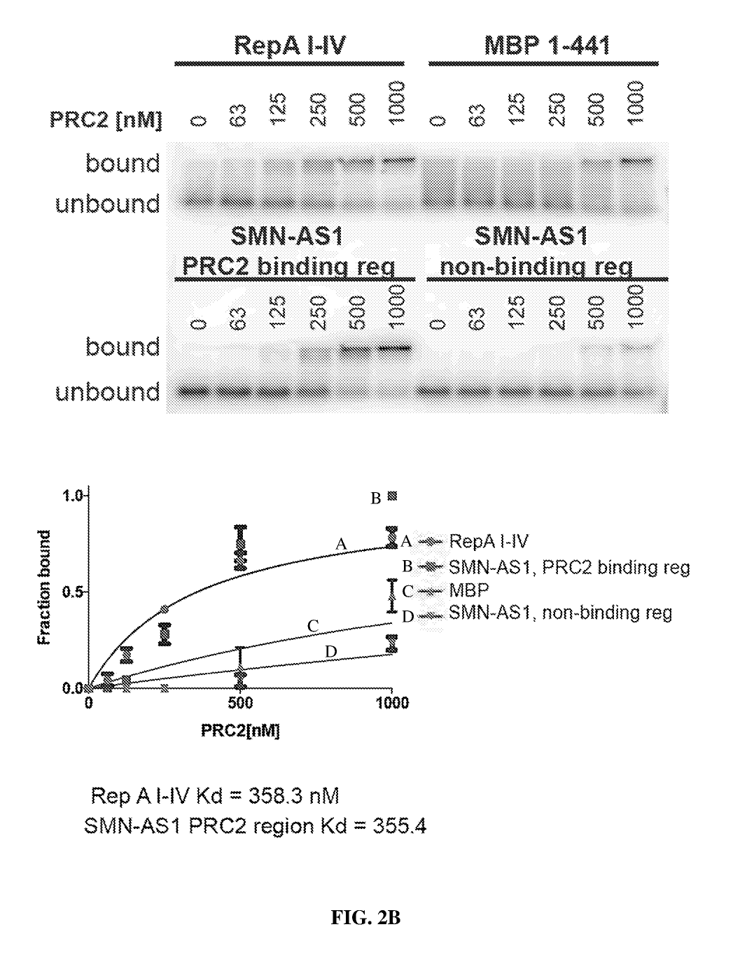

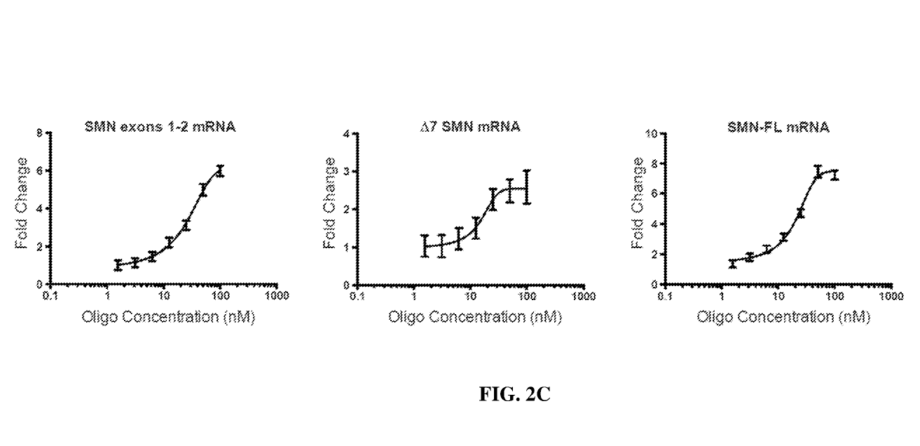

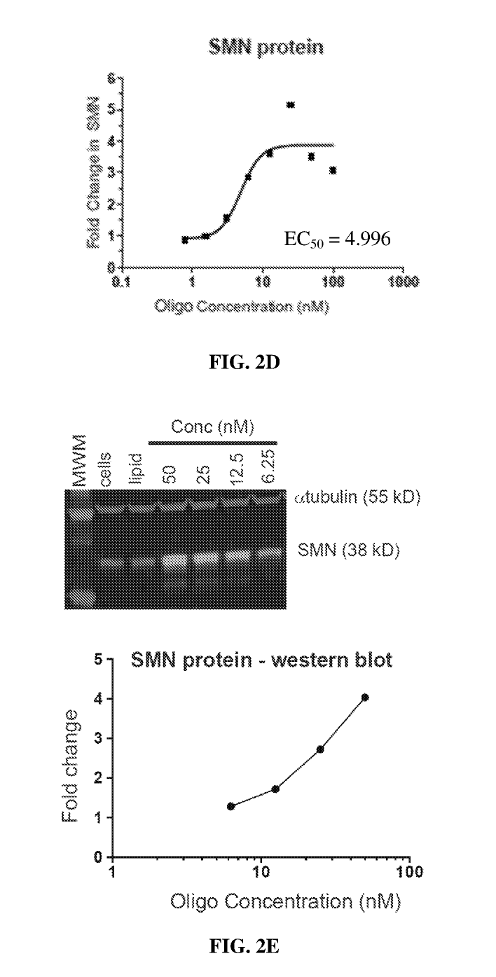

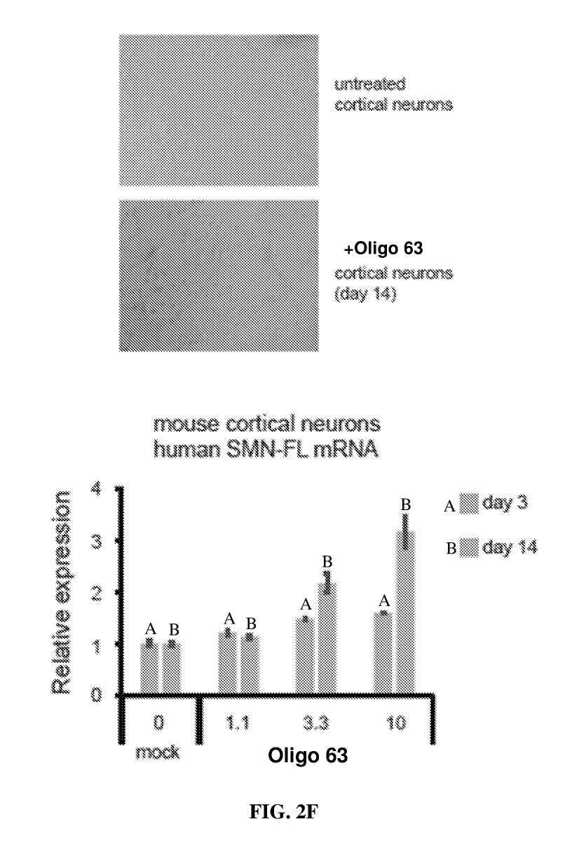

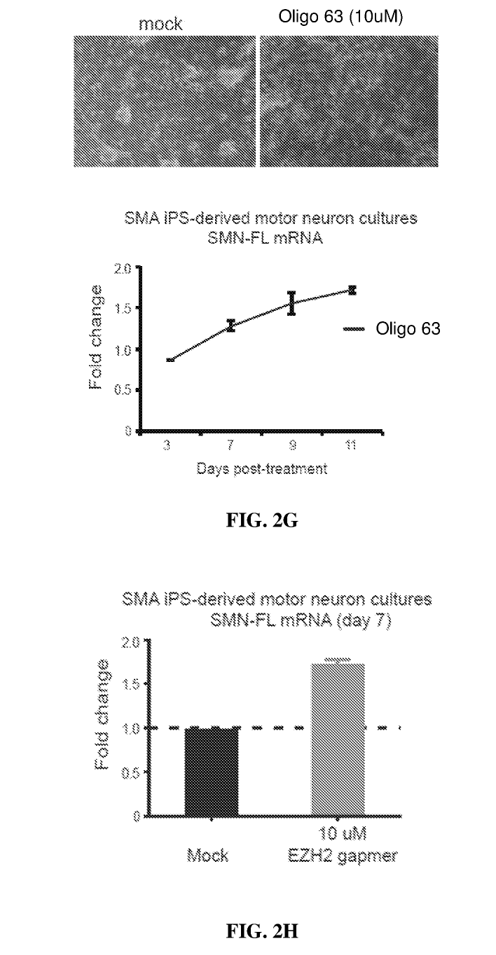

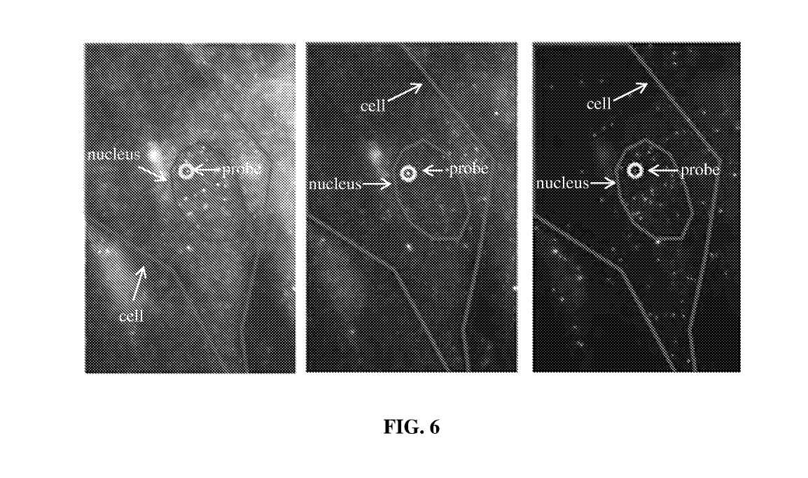

LncRNAs recruit chromatin-modifying complexes via direct interactions to regulate gene expression. Native RNA immunoprecipitation (RIP) using an antibody to SUZ12, a component of the PRC2 complex, demonstrated that SMN-AS1 was associated with PRC2 in SMA fibroblasts (FIG. 2A). In support of this finding, SMN-AS1 was also associated with EZH2 by RIP (FIG. 5). The interaction with SMN-AS1 is strand-specific, as SMN mRNA did not show significant association with PRC2. ANRIL, a lncRNA that recruits PRC2 to act in cis at the p15/CDKN2A gene is a positive control for this assay. Highly expressed RNAs, such as 18S and GAPDH were not found to be associated with PRC2. Short steric-blocking oligonucleotide “mixmers” consisting of locked nucleic acids were interspersed with 2′-O-methyl nucleotides were designed to hybridize to potential sites of interaction along SMN-AS1 to sterically block PRC2 association based on RIP-sequencing data. These mixmers are designed to bind to the target RNA with high affinity but do not trigger the RNase H pathway for exonuclease degradation. As a result, SMN-AS1 is still present at the SMN locus (FIG. 6). SMA fibroblasts were transfected with the oligonucleotide Oligo 63, and RIP experiments showed significantly less SMN-AS1 associated with PRC2 (p=0.006) (FIG. 2A). In contrast, a mixmer targeting SMN-AS1 that was not identified in this context as a direct interaction site with PRC2 (Oligo 52) did not change their association. The effect of Oligo 63 was specific to the SMN-AS1/PRC2 interaction since it did not alter the association of PRC2 with SMN mRNA, ANRIL, GAPDH, and 18S. Taken together, this steric-blocking mixmer was used to selectively prevent PRC2 from interacting with a target lncRNA without interfering with other PRC2/lncRNA interactions. The structure of Oligo 63 follows: lnaCs; omeAs; lnaT; omeAs; lnaGs; omeUs; lnaGs; omeGs; lnaAs; omeAs; lnaCs; omeAs; lnaGs; omeAs; lnaT, in which ome indicates a 2′-OMe (O-Methyl), lna indicates an LNA, s indicates a 3′ thiophosphate, A is adenine, T is thymine, U is uridine, C is cytosine, and G is guanine.

RNA elcctromobility shift assays (EMSA) was also performed to determine whether the interaction between a purified recombinant human PRC2 complex and the SMN-AS lncRNA is direct (FIG. 2B). A 444-nucleotide region of SMN-AS1 containing the PRC2 interaction site (PRC2 region) was combined with the PRC2 complex consisting of EED, SUZ12, and EZH2 and observed the RNA shifted to the bound fraction. However, another 444-nucleotide region of SMN-AS1 that did not appear to interact directly with PRC2 by RIP-sequencing (SMN-AS1, neg region) also did not show a concentration-dependent protein-RNA mobility shift. The RepA region of Xist directly interacts with PRC2 and is required for PRC2 recruitment to the X chromosome for human X inactivation. The 441-nucleotide RepA region used in the EMSA showed a shift when hound to PRC2. MBP1, an RNA known not to bind PRC2 also did not show any specific interactions with PRC2 in the assay. When Kd of the fraction of RNA bound by PRC2 was calculated, the SMN-AS1/PRC2 interaction site bound to PRC2 with similar affinity as the RepA region of Xist, whereas the negative control region of SMN-AS1 and MBP1 did not (FIG. 2B). These data provide evidence that there are specific and direct interactions between PRC2 and the SMN-AS lncRNA.

Because Oligo 63 had the effect of blocking the interactions between SMN-AS1 and PRC2, whether this resulted in changes in SMN mRNA expression was examined. SMA fibroblasts transfected with Oligo 63 showed increases in exon 7-inclusive (SMN-FL) SMN mRNA as well as exon 7 excluding (SMNΔ7) transcripts in a concentration-dependent manner (FIG. 2C). Overall SMN mRNA levels were measured with primers that span exon 1 and exon 2 to measure all isoforms and observed a 6-fold increase over the baseline levels. The degree of increase for SMNΔ7 mRNA was approximately 2.5-fold and the increase in SMN-FL mRNA was 7-fold over baseline levels. This non-proportional increase may arise from increased functional SMN protein leading to more effective splicing at the SMN2 gene itself or PRC2 playing a role in splicing. Indeed, at the FGFR2 locus, PRC2 is recruited to play a role in differential splicing at an actively transcribed gene. Determining whether the larger relative increase in SMN-FL mRNA is due to changes in splicing associated with changes in PRC2 activity and/or the SMN-containing splicing activity on the gene itself is difficult to distinguish. However, because the overall levels of SMN mRNA have increased, the primary effect of Oligo 63 is likely due to increased transcription. In agreement with this, SMN protein levels also increased when the SMA fibroblasts were treated with Oligo_63. The ELISA shows a concentration-dependent increase in SMN protein levels on day 5 correlating with increased SMN mRNA levels (FIG. 2D). Western blot were performed to determine which SMN isoforms changed upon oligo treatment. The results show an increase primarily of the 38-kd SMN-FL protein in response to an increase in Oligo 63 treatment up to 4-fold above the lipid or untreated SMA fibroblasts (FIG. 2E).

RNA-sequencing on SMA fibroblasts treated with Oligo 63 was performed to determine the specificity of the treatment. Overall, there were very few changes in gene expression. Indeed, when these results were compared with data from SMA fibroblasts treated with a splice-switching oligo that works directly on the SMN pre-mRNA to include exon 7, many of the gene expression changes observed with Oligo 63 treatment were also observed with the splice-switching oligo treatment. The similar gene expression profiles arising from independent mechanisms to increase SMN-FL suggests that the expression changes may be due to increased functional SMN.

To assess the activity of Oligo 63 in a terminally differentiated disease-relevant cell type, cortical neurons were prepared ex vivo from E15/E16 embryos of wild-type littermates of the Δ7 mouse model, harboring 2 copies of the human SMN2 transgene. The cortical neurons were treated gymnotically with Oligo 63 for 14 days resulting in no phenotypic changes (FIG. 2F). Although by day 3, no significant changes were seen, by day 14, a concentration-dependent increase in human SMN-FL mRNA levels was observed. No changes were observed in the mouse smn mRNA levels from the cortical neurons at any time point because Oligo 63 is not predicted to hind to the mouse smn antisense lncRNA, providing further evidence that Oligo 63 acts specifically on the human SMN-AS1/PRC2 interaction. The delayed increase in human SMN-FL mRNA levels, relative to the increases observed 2 days after transfection, may be partially due to the bioavailability available after unassisted delivery and/or due to the non-proliferating state of the cells. Indeed, the rate of H3K27me3 removal from chromatin of non-dividing cells is slower than in proliferating cells. In addition, iPS-derived motor neuron cultures from SMA patients were treated with Oligo 63 over an 11-day period. Consistently, SMN-FL mRNA increased over time compared to the untreated neuronal cultures in which the mRNA levels remained constant (FIG. 2G). Taken together, these results indicate that steric-blocking mixmers can effectively increase SMN-FL mRNA levels in neuronal cell types relevant to SMA.

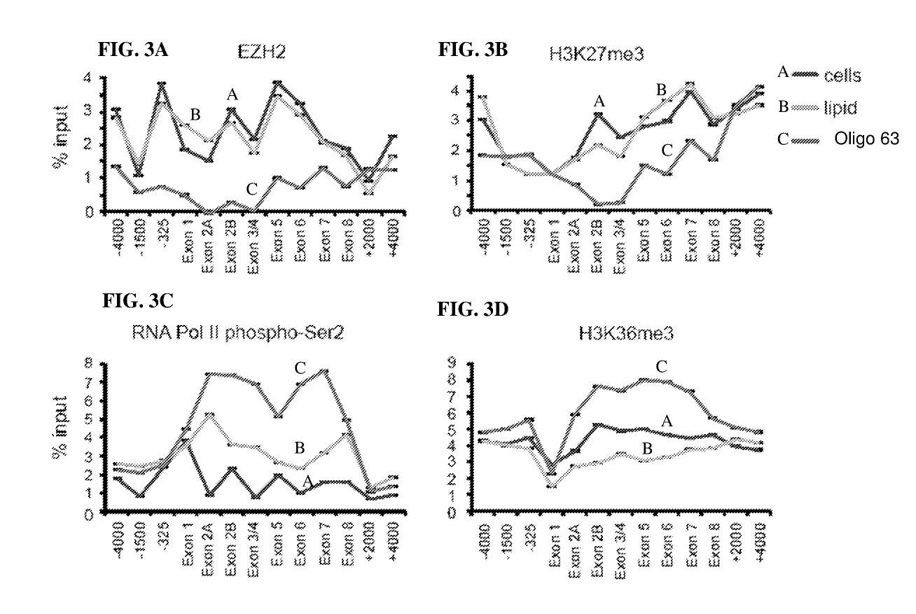

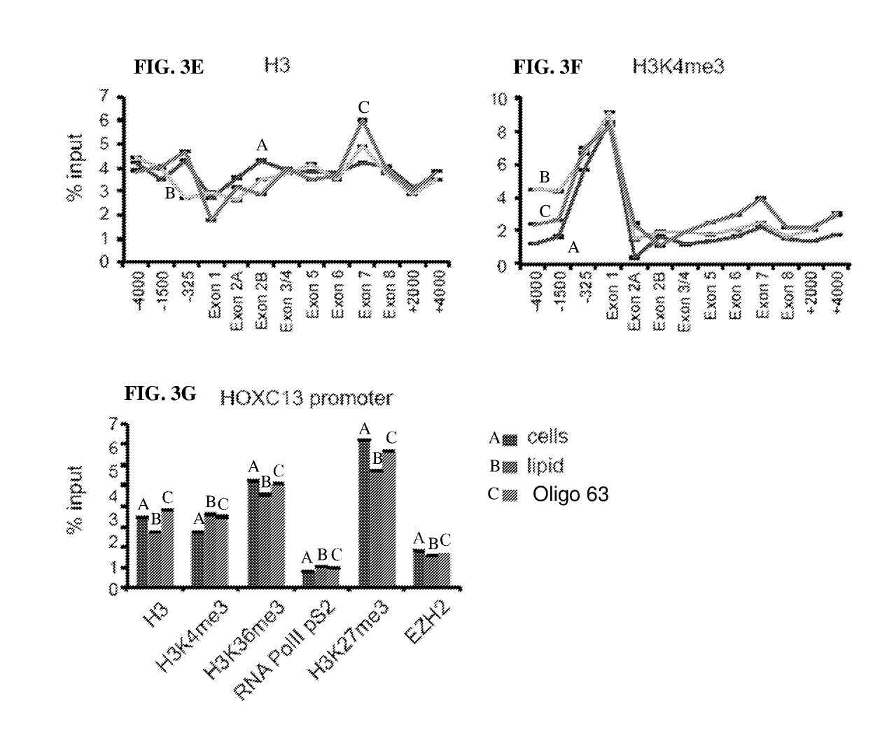

Chromatin immunoprecipitation (ChIP) was used to look at possible changes in chromatin at the SMN locus when SMA fibroblasts were treated with Oligo_63. Oligo 63 treatment resulted in the loss of EZH2 associated with the SMN gene body as well as decreased H3K27me3 levels (FIGS. 3A-3B). Concomitantly, there was an increase in the transcriptionally elongating form of RNA Polymerase II, phosphorylated at serine 5 along the CTD, and increased levels of the H3K36me3 mark of transcriptionally active chromatin associated with the SMN locus (FIGS. 3C-3D). The loss of PRC2 at a transcriptionally active gene, along with the increase in RNA polymerase II occupancy and H3K36me3 levels suggest that chromatin changes favoring increased transcription allow for increased amounts of SMN mRNA and protein. H3 levels were similar amongst all samples (FIG. 3E). The peak of H3K4me3 at the promoter of SMN did not show changes at this already actively transcribed gene (FIG. 3F). In addition, no changes were observed in PRC2 association with the HOXC13 promoter upon Oligo 63 treatment (FIG. 3G), supporting the RNA-sequencing results showing selectivity of Oligo_63.

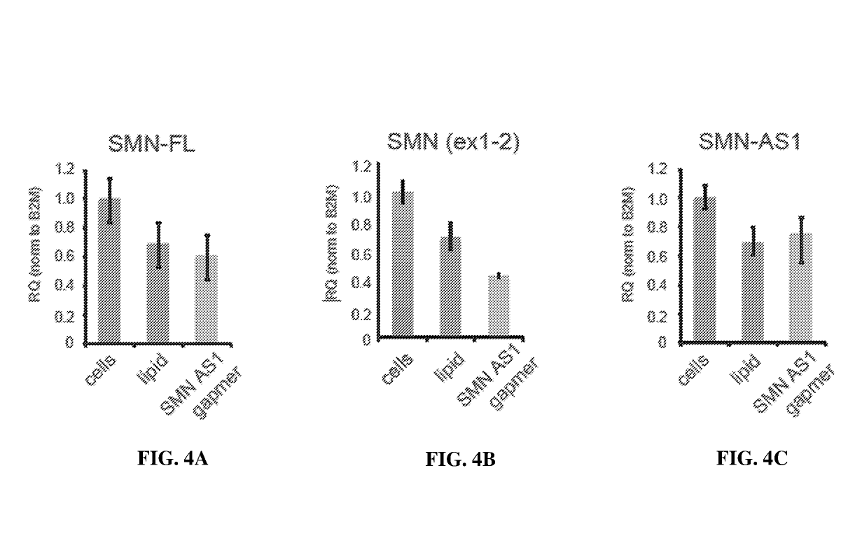

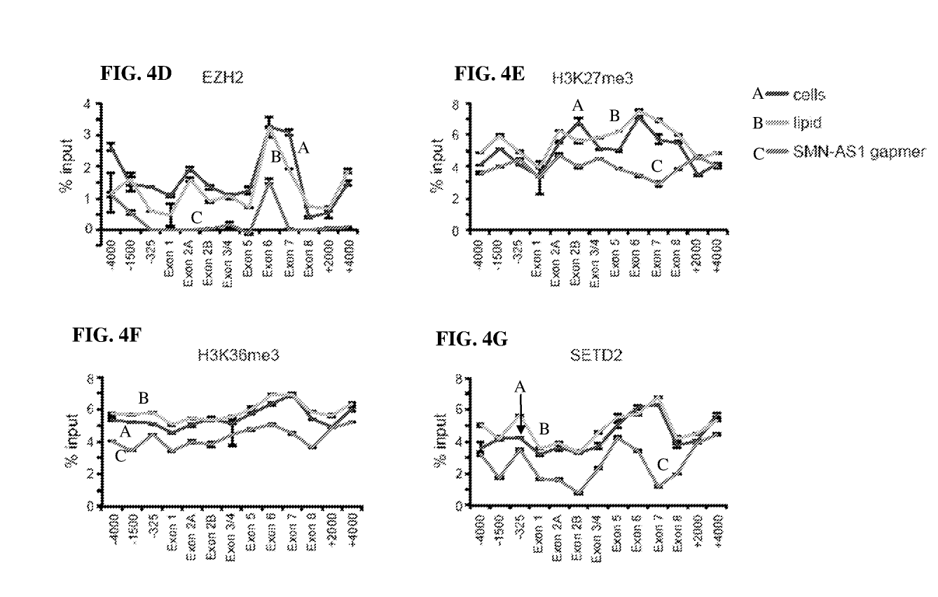

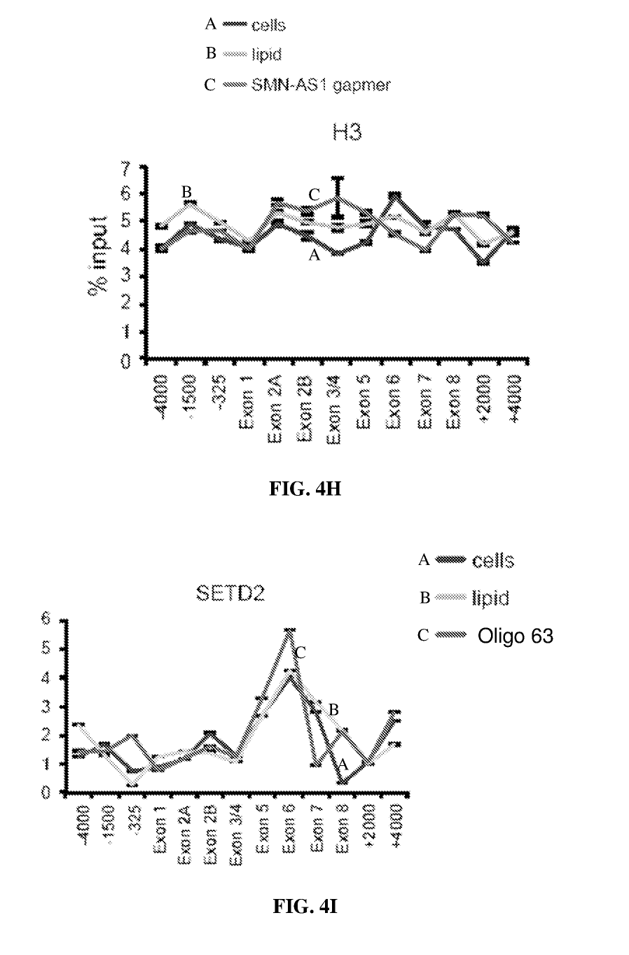

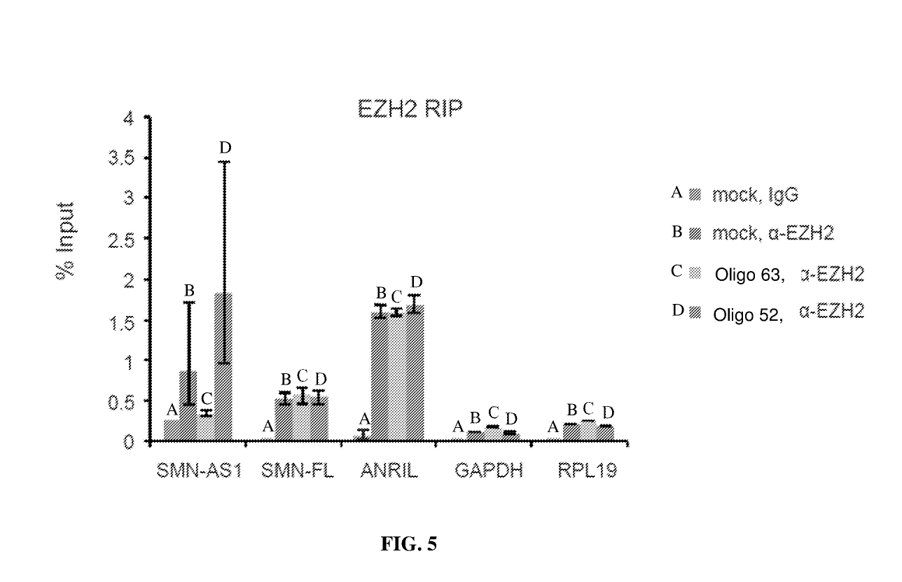

When the SMN-AS1 lncRNA was knocked down with a SMN-AS1 gapmer, an increase in SMN mRNA levels was not observed (FIGS. 4A-4C). ChIP results showed the loss of PRC2 and H3K27me3 levels (FIG. 4D). However, the knockdown of SMN-AS1 led to a decrease in H3K36me3 levels (FIG. 4D) and no increase in RNA polymerase II. Whether the H3K36me3 methyltransferase SETD2 might be affected was tested. Indeed, SMN-AS1 lncRNA knockdown resulted in the loss of SETD2 association with the gene body. RIP demonstrated that SETD2 normally is associated with the SMN-AS lncRNA and treatment with Oligo 63 does not affect SETD2 association. Taken together, SMN-AS1 lncRNA recruits both repressive PRC2 and activating SETD2 complexes to the SMN locus to modulate levels of transcription. By selectively blocking PRC2 recruitment to the SMN locus, Oligo 63 is able to maintain the recruitment of SETD2, thereby increasing transcription, indicating that SMN-AS lncRNA functions as a scaffold (a non-coding RNA scaffold) for both an activator (SETD2) and a repressor (PRC2).

The severity of SMA, in general, inversely correlates with the number of copies of the SMN2 gene. It is believed that the fraction of SMN-FL that arises from SMN2 contributes to the survival of spinal motor neurons but that insufficient levels of SMN eventually lead to cell death. If SMN transcription can be upregulated from the existing copies of SMN2 that a patient has, there may be enough SMN-FL mRNA and functional SMN protein produced to confer therapeutic benefit. In parallel, the discovery of a PRC2-based mechanism that regulates SMN gene expression provided the opportunity to demonstrate that endogenous mRNA levels can be increased by specifically blocking PCR2 activity at a target gene using a SB mixmer to disrupt PRC2 recruitment via a lncRNA. This novel approach provides specificity to modifying an epigenetic mechanism that regulates many key developmental and regulatory genes.

### Example 2

Gene Activation of SMN by Selective Disruption of lncRNA Recruitment of PRC2 for the Treatment of Spinal Muscular Atrophy

### Summary

Spinal muscular atrophy (SMA) is a neurodegenerative disease characterized by progressive motor neuron loss and caused by mutations in SMN1 (Survival of Motor Neuron 1). Currently, there is no disease-modifying therapy and the disease severity inversely correlates with the copy number of SMN2, a duplicated gene that is nearly identical to SMN1. The present disclosure delineates a novel mechanism of transcriptional regulation in the SMN2 locus. A previously uncharacterized long noncoding RNA, SMN-AS1, represses SMN2 expression by recruiting Polycomb Repressive Complex 2 (PRC2) to its locus. Using sterically blocking oligonucleotides to disrupt the interaction between SMN-AS1 and PRC2, the recruitment of PRC2 is inhibited while SMN2 expression is increased in primary neuronal cultures. Proof-of-concept evidence that SMA may be treatable by applying a novel gene-upregulation technology is demonstrated herein. Additionally, these data suggest that such approach can selectively upregulate genes that are epigenetically repressed by lncRNA and PRC2.

### Introduction

Polycomb Repressive Complex 2 (PRC2) is a histone methyltransferase complex that plays essential roles in development and disease (Di Croce and Helin, 2013; Simon and Kingston, 2013; Kadoch et al., 2016). Mammalian PRC2 is composed of four obligatory subunits, EED, SUZ12, RbAp48, and EZH1 or EZH2. EZH1 and EZH2 are the histone methyltransferases that confer the trimethylation of lysine 27 of histone H3 (H3K27me3) and PRC2-mediated H3K27me3 is associated with the maintenance of gene repression (Simon and Kingston, 2013). The formation of an EZH1- or EZH2-containing PRC2 complex depends on chromosomal location and cell type (Margueron et al., 2008). On its own, the core PRC2 complex does not contain any sequence-specific DNA binding activity. However, it interacts with other DNA-binding subunits in a substoichiometric manner and is recruited to specific Polycomb Response Elements (PREs) (Vizan et al., 2015). Despite only a few mammalian PREs identified to date (Sing et al., 2009; Woo et al., 2010, 2013; Basu et al., 2014), emerging data suggests that PRC2 interacts with a large number of RNA transcripts (Zhao et al., 2010; Davidovich et al., 2015) and a subset of which may aid PRC2 recruitment to specific genomic locations (reviewed by Davidovich and Cech, 2015) to repress the expression of neighboring genes. Combining a functional genomic approach and in-depth mining of epigenetic databases led to the identification of PRC2-regulated genes including SMN2, a disease-modifying gene for SMA.

Spinal Muscular Atrophy (SMA) is the leading genetic cause of infant mortality, caused by deletions or mutation of SMN1 which gene product is critical for mRNA processing (Rossoll et al., 2002). SMN1 is uniquely duplicated in the human genome and yields SMN2, which is nearly identical in sequence. However, a C-to-T point mutation in exon 7 of SMN2 results in preferential skipping of exon 7 during pre-mRNA splicing and production of a truncated and unstable protein. A small fraction (10-20%) of pre-mRNA transcribed from SMN2 is spliced correctly to include exon 7 and produces a full-length SMN (SMN-FL, inclusive of exon 7) that is identical to the SMN1 gene product (Monani, 2005; Vitte et al., 2007).

Spinal motor neurons are highly sensitive to SMN1 deficiency and their premature death causes motor function deficit in SMA patients (Monani, 2005; Burghes and Beattie, 2009). The SMN2-derived SMN-FL mRNA can extend spinal motor neuron survival yet insufficient level of SMN-FL mRNA eventually leads to cell death. Clinically, SMA patients who have increased SMN2 genomic copy number have a less severe disease phenotype (Lefebvre et al., 1997; Feldkotter et al., 2002). Therefore, it was thought that increasing SMN2 transcription could phenocopy the beneficiary effect of SMN2 gene amplification and compensate for SMN1 deficiency. In addition, SMN1 heterozygotes are asymptomatic while affected homozygotes have 10-20% of normal SMN levels, so it was predicted that a modest SMN2 upregulation would provide significant therapeutic benefit. Here, it is established that PRC2 interacts with a newly identified long noncoding RNA (lncRNA) transcribed within the SMN2 locus and regulates SMN2 expression through PRC2-associated epigenetic modulation. Furthermore, the selective upregulation of SMN2 expression by sterically blocking the lncRNA-mediated recruitment of PRC2 to the SMN2 locus is demonstrated.

### Results

### PRC2 Modulates SMN2 Expression

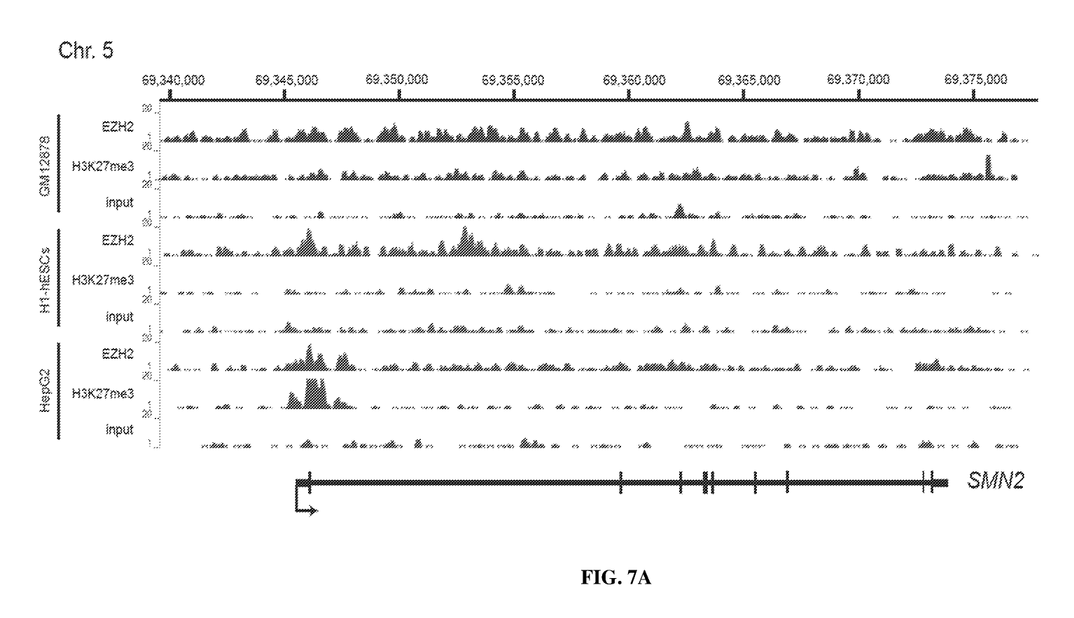

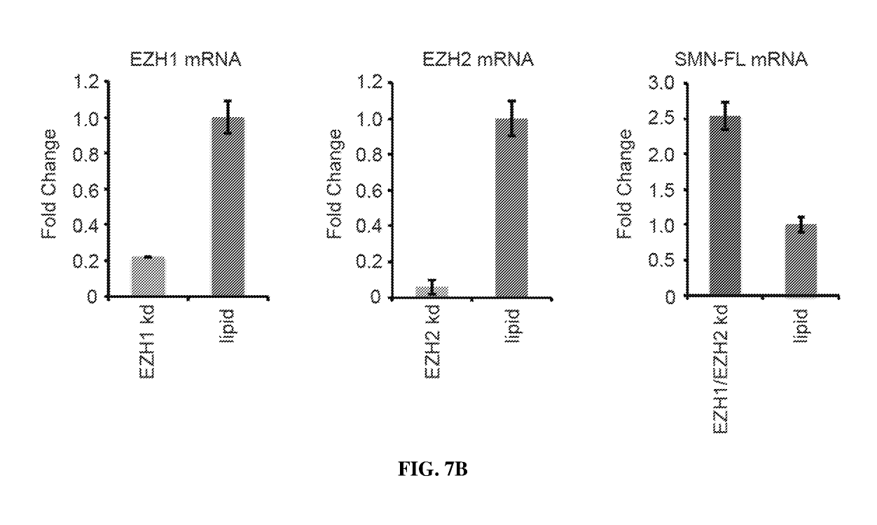

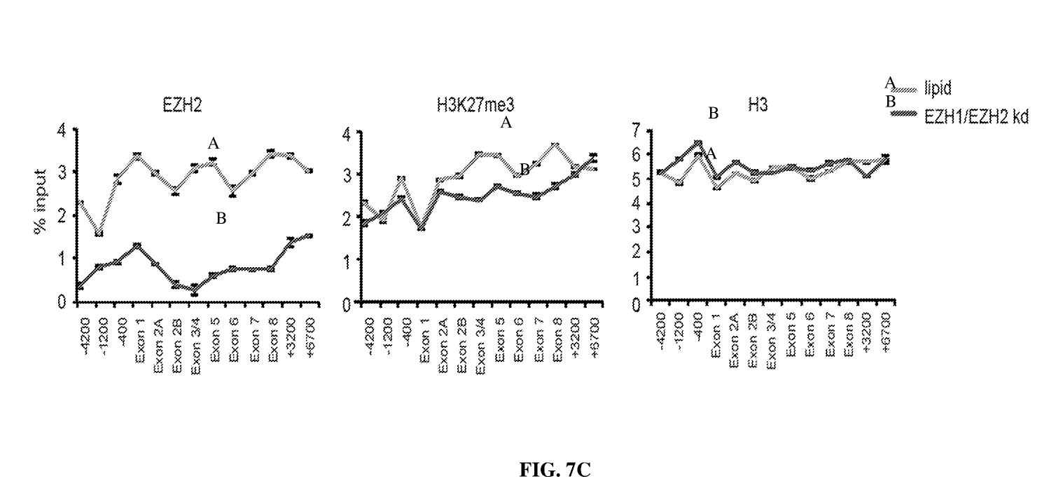

In depth and focused analysis of publically available chromatin immunoprecipitation (ChIP) sequencing data suggests that PRC2 is associated with SMN2 in multiple cell types (FIG. 7A). Enriched EZH2 interaction and its associated repressive chromatin marks, H3K27me3, suggest that PRC2 activity is targeted to the gene. To determine whether disruption of PRC2 activity could lead to increases in SMN2 expression, EZH1 and EZH2 mRNAs were knocked down in SMA fibroblasts using antisense oligonucleotide (ASO) designed for RNaseH-mediated degradation. Two days post-transfection, there were significant decreases in EZH1 and EZH2 mRNA levels. Knockdown of both EZH1 and EZH2 in the SMA fibroblasts was associated with an increase in SMN-FL mRNA (FIG. 7B). The SMN1 and SMN2 loci (from here on collectively termed “SMN locus”) were further analyzed for chromatin changes upon EZH1/EZH2 knockdown through ChIP. Because SMN1 and SMN2 have >99% sequence identity (27,890 of 27,924 basepair match), it is not possible to distinguish between the two genes using this technique. The decreased association of EZH2 as well as decreased H3K27me3 levels at the locus were observed, without any changes in total H3 (FIG. 7C). This suggests that PRC2 directly regulates the expression of SMN.

### Identification of SMN-AS1 at the SMN Locus

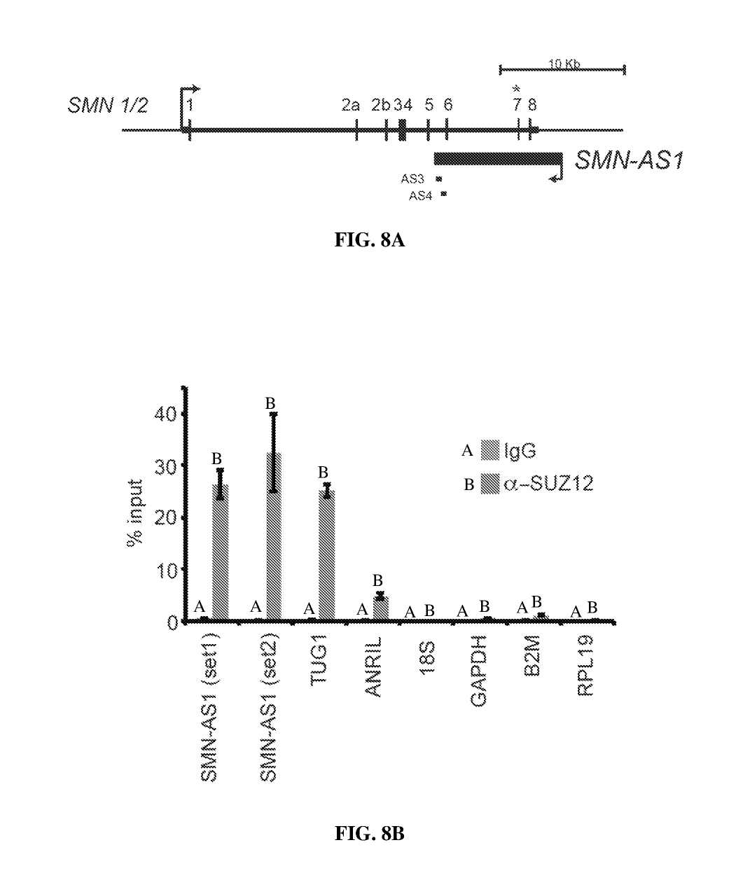

Detailed analysis of RNA immunoprecipitation (RIP)-seq datasets revealed a previously undescribed PRC2 interacting antisense RNA within the mouse Smn locus (Zhao et al., 2010). Whether the antisense transcript exists in human and may have a role in PRC2-mediated SMN repression was investigated. Next generation RNA-sequencing revealed that a lncRNA, SMN-AS1, is transcribed from the SMN loci (FIGS. 8A, 1C). Due to the high sequence identity between the SMN1 and SMN2 loci, the lncRNA, SMN-AS1 was expected to be transcribed from both loci. Furthermore, its expression in both SMN1- or SMN2-mutated cell lines was observed (FIG. 1C). Northern blot analysis of human fetal brain and adult lung tissues revealed that SMN-AS1 is up to 10 kb long, is heterogeneous in size, and has differential expression between the two tissue types (FIG. 1B). To confirm the specificity of the SMN-AS probe, a humanized SMA mouse model carrying two copies of the human SMN2 genomic locus (5025 strain) was used (Le et al., 2005). Comparing the brain tissues from wild type and 5025 mice, a similar set of transcripts in the SMN2-harboring transgenic mice and in the human fetal brain were observed (FIG. 1B). By reverse transcription quantitative PCR (RT-qPCR), SMN-AS1 was detected in patient cell lines and the level of expression correlated with SMN2 copy number (FIG. 1C). In addition, it was found that SMN2 mRNA and SMN-AS1 expression is highly correlated with CNS tissues (FIG. 1D). Finally, strand-specific single-molecule RNA-fluorescent in situ hybridization (RNA-FISH) detected the SMN-AS1 at the SMN locus (FIG. 1E). Together, these data demonstrate the presence of an antisense transcript within the SMN locus.

### SMN-AS1 Binds PRC2

To investigate the role of SMN-AS1 in the PRC2-mediated epigenetic regulation of the SMN2 gene, native RIP (nRIP) was performed using an antibody against the PRC2 subunit, SUZ12, followed by RT-qPCR with 2 distinct probe sets directed to different regions of SMN-AS1. RIP-qPCR showed that SMN-AS1 is strongly associated with PRC2 in SMA fibroblasts (FIG. 8B). The association was stronger than, or comparable to, that of well-established PRC2 interacting lncRNAs including TUG1 (Zhang et al., 2014) and ANRIL (Kotake et al., 2011). Additionally, PRC2 did not associate with the abundantly expressed negative controls such as GAPDH and RPL19. Similar results were observed with the nRIP for EZH2 (FIG. 5) further supporting the association of SMN-AS1 with PRC2. Because nRIP identifies both direct and indirect interactions, RNA electrophoretic mobility shift assays (RNA EMSA) were performed to specifically detect direct interactions. Using a 441-nucleotide (nt) RNA containing the PRC2 interacting region of SMN-AS1 (SMN-AS1, PRC2 binding region) as identified by RIP-seq (Zhao et al., 2010), it was observed that purified recombinant human PRC2 (EED/SUZ12/EZH2) specifically changed the migration of this region of SMN-AS1 (FIG. 2B). Binding was concentration-dependent and was as robust as that of the 434-nt RepA RNA, a conserved domain of Xist RNA that is a well-documented PRC2-interacting lncRNA (Zhao et al., 2010; Cifuentes-Rojas et al., 2014). Dissociation constants (Kd) of both transcripts were estimated to be 350-360 nM. As specificity controls, a low level of background binding to a non-PRC2 interacting 441-nt region of the SMN-AS1 transcript (SMN-AS1, non-binding region) and to another non-specific mRNA of similar length, maltose-binding protein (MBP) from E. coli (Cifuentes-Rojas et al., 2014) was observed. These data demonstrate that SMN-AS1 lncRNA interacts directly and specifically with PRC2.

### Blocking PRC2:SMN-AS1 Interaction Upregulates SMN2 and Produces Epigenetic Changes

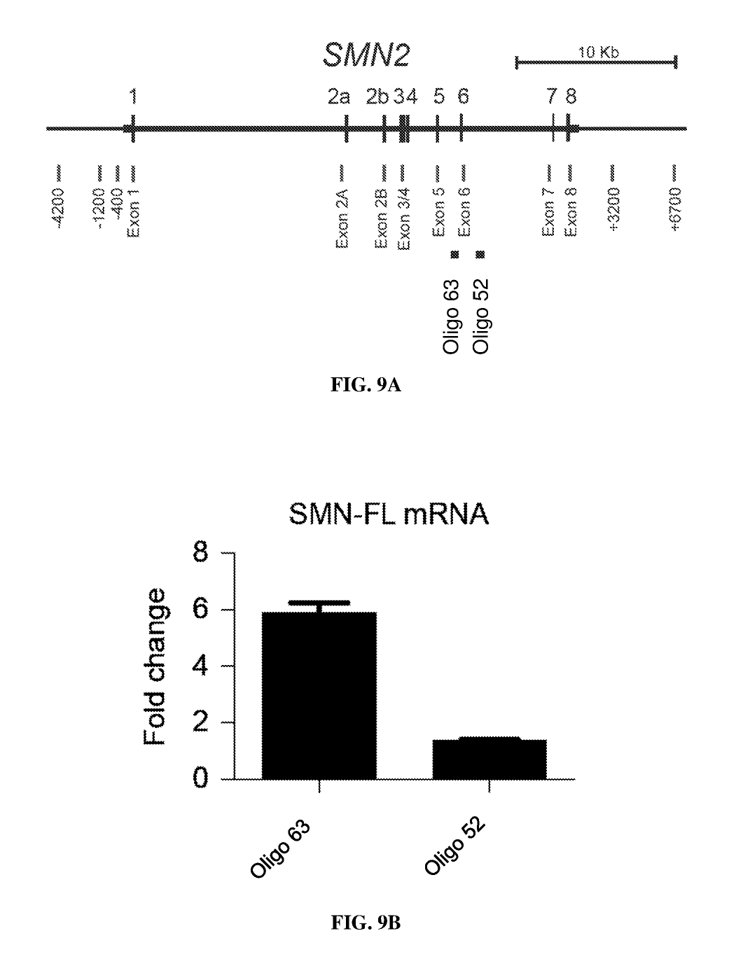

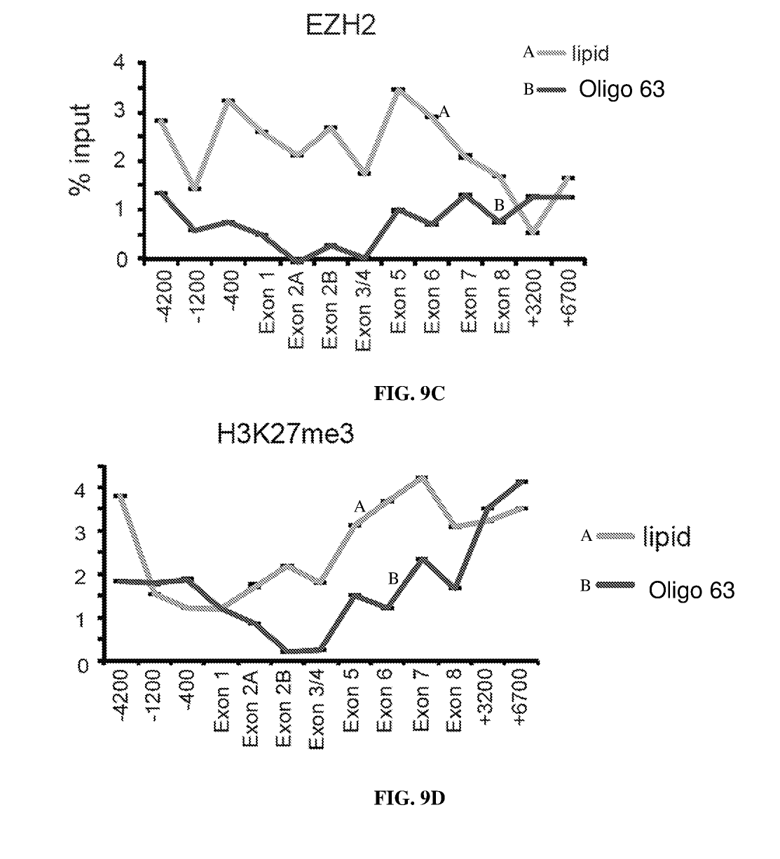

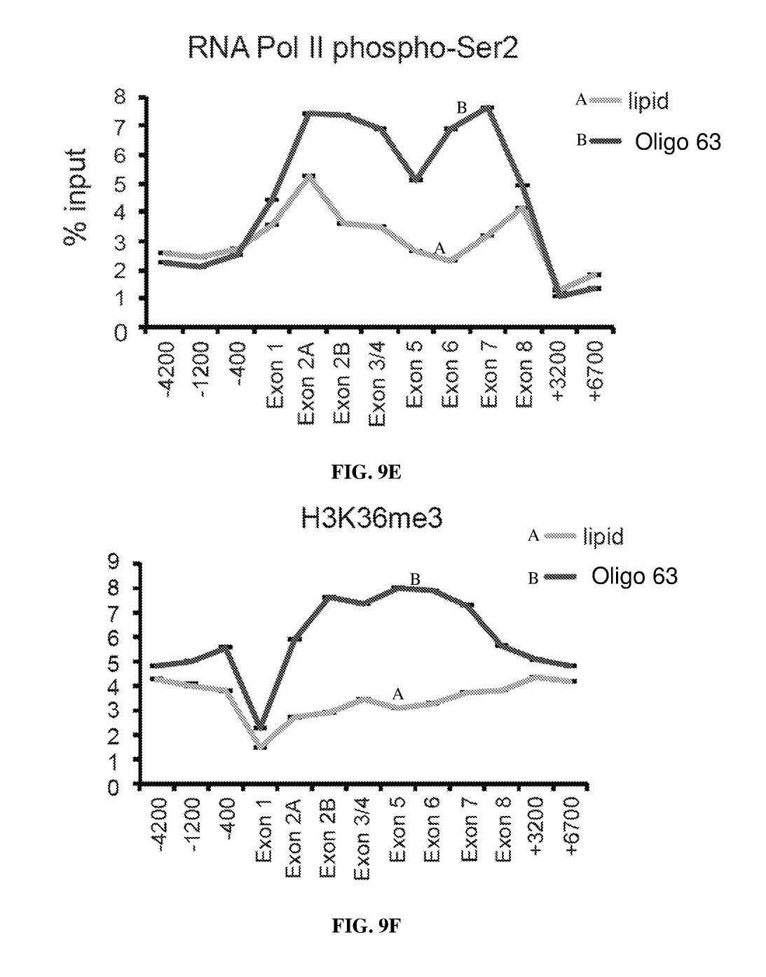

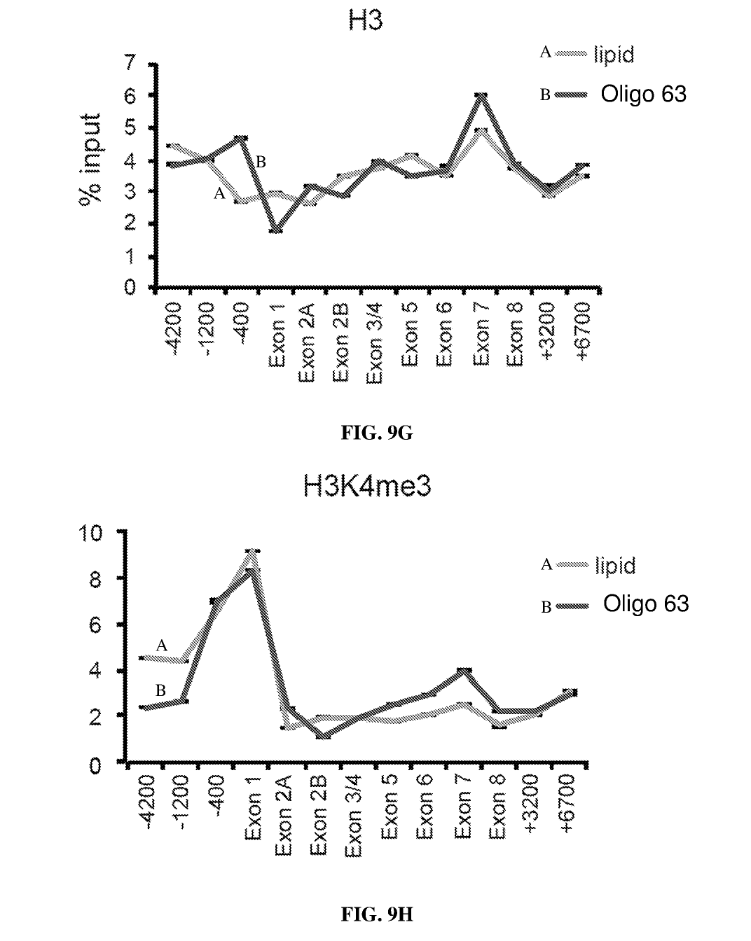

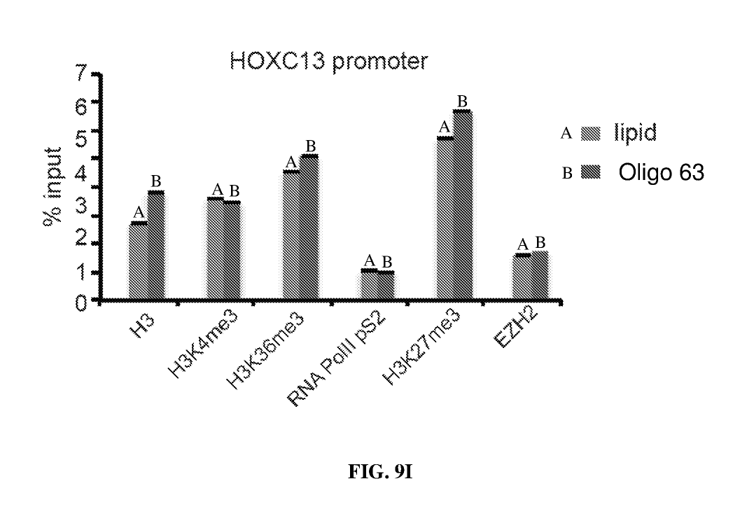

To investigate the effect of disrupting PRC2:SMN-AS1 interaction, ASOs targeting the PRC2-binding site of the lncRNA were designed. ASOs hybridize to target RNA sequences via Watson-Crick complementarity pairing. Depending on the arrangement of DNA- and LNA-modified nucleotides, such interaction can lead to either RNaseH-mediated degradation of target RNAs or hindrance of the interaction between target RNAs and their binding partners. For RNaseH-mediated degradation, a “gapmer” formatted ASO composed of a central DNA segment greater than 6 nucleotides (i.e. gap) flanked by 2 to 4 locked nucleic acid (LNA)-modified nucleotides is required. These gapmer ASOs were used to knockdown EZH1 and EZH2 in earlier experiments (FIG. 7C). In contrast to the gapmer arrangement, a “mixmer”-formatted ASO lacks the central DNA segment and does not support the RNaseH-mediated degradation mechanism. Instead, the binding of a mixmer ASO prevents the interaction between target RNA and its RNA or protein binding partners (Kauppinen et al., 2005). Mixmer ASOs consisting of LNA interspersed with 2′-O-methyl nucleotides (2′-OMe) for high-affinity binding to SMN-AS1 were generated. Screening multiple mixmer ASOs led to a focus on one efficacious mixmer ASO, Oligo 63 (FIG. 9A). Transfecting Oligo 63 into SMA fibroblasts significantly increased SMN-FL expression where as transfecting with another mixmer ASO, Oligo 52, which was not predicted to sterically block PRC2 recruitment, did not change SMN-FL expression (FIG. 9B). Consistently, nRIP showed that Oligo 63, but not Oligo 52, disrupted the binding of PRC2 to SMN-AS1, as shown by RIP-qPCR (FIG. 9B). Furthermore, no effect of Oligo 63 or Oligo 52 was observed on ANRIL, GAPDH, or RPL19 control RNAs. These results were also observed when the nRIP was performed using an antibody against EZH2 (FIG. 5). As expected, single molecule RNA-FISH for the localization of SMN-AS1 after transfection with Oligo 63 showed no change in both the abundance and the localization of SMN-AS1 in 93% of cells examined (39 of 42 nuclei) (FIG. 6). Together, these results demonstrate that selective inhibition of PRC2:SMN-AS1 interaction by a mixmer ASO leads to increase SMN2 expression.

To provide molecular insight on how the active mixmer induced SMN expression, the chromatin changes at the SMN locus in response to the disruption of PRC2:SMN-AS1 interaction was characterized using ChIP analysis. When treated with Oligo 63, a loss of EZH2 association as well as decreased H3K27me3 levels, the histone mark associated with PRC2 activity with the SMN gene body, were observed (FIGS. 9C-9D). Thus, the mixmer ASO could indeed block the recruitment and activity of PRC2 at the SMN2 locus. Concomitantly, there was an increase in binding of RNA Pol II-phosphoSer2 (RNA Polymerase II, phosphorylated at serine 2) and elevated levels of H3K36me3, both of which indicate transcriptional elongation (FIGS. 9E-9F). By contrast, pan-H3 levels were similar amongst all samples (FIG. 9G). Moreover, H3K4me3, a mark of transcription initiation, did not change at the promoter (FIG. 9H), suggesting that the regulation is occurring at the transcriptional elongation level. No changes in PRC2 association were observed at another well-established Polycomb target locus, HOXC13, upon treatment (FIG. 9I). It was concluded that PRC2 recruitment and activity at the SMN locus can be selectively inhibited by sterically blocking the specific PRC2:SMN-AS1 interaction.

SETD2 Interacts with SMN-AS1 to Promote Transcription

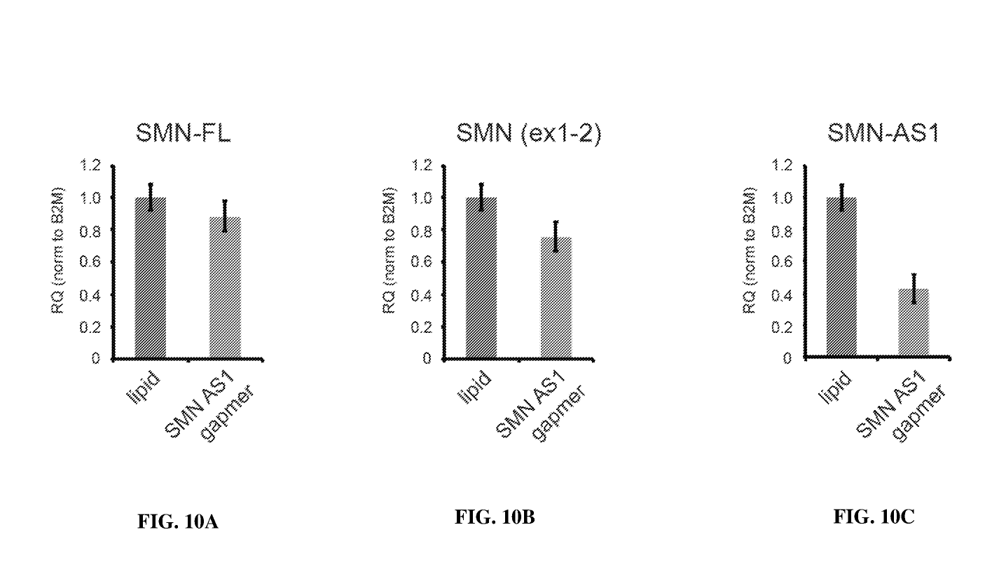

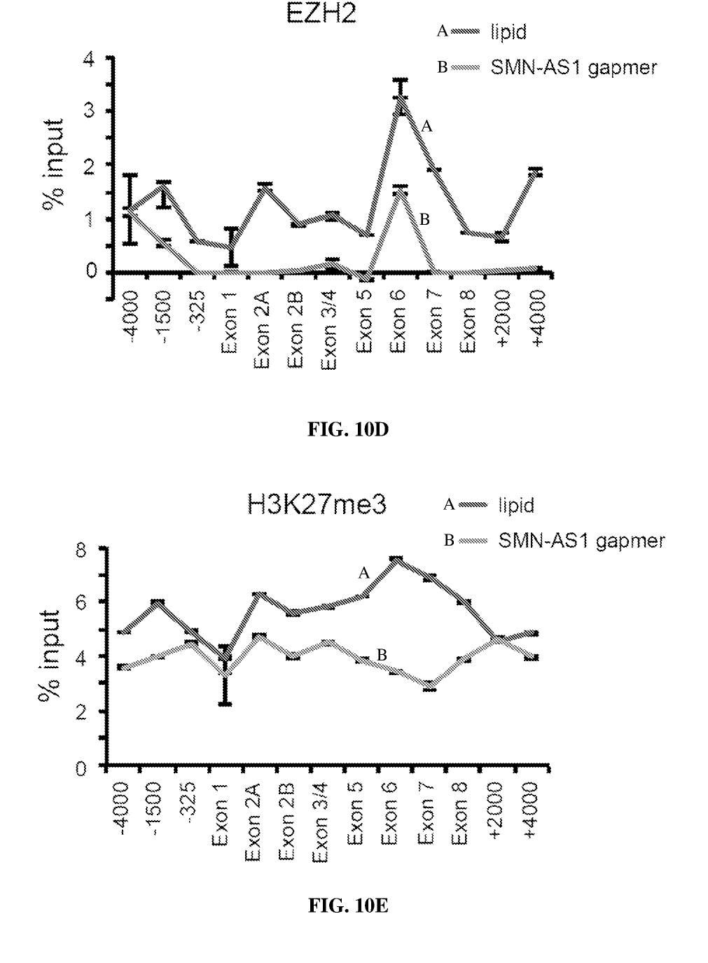

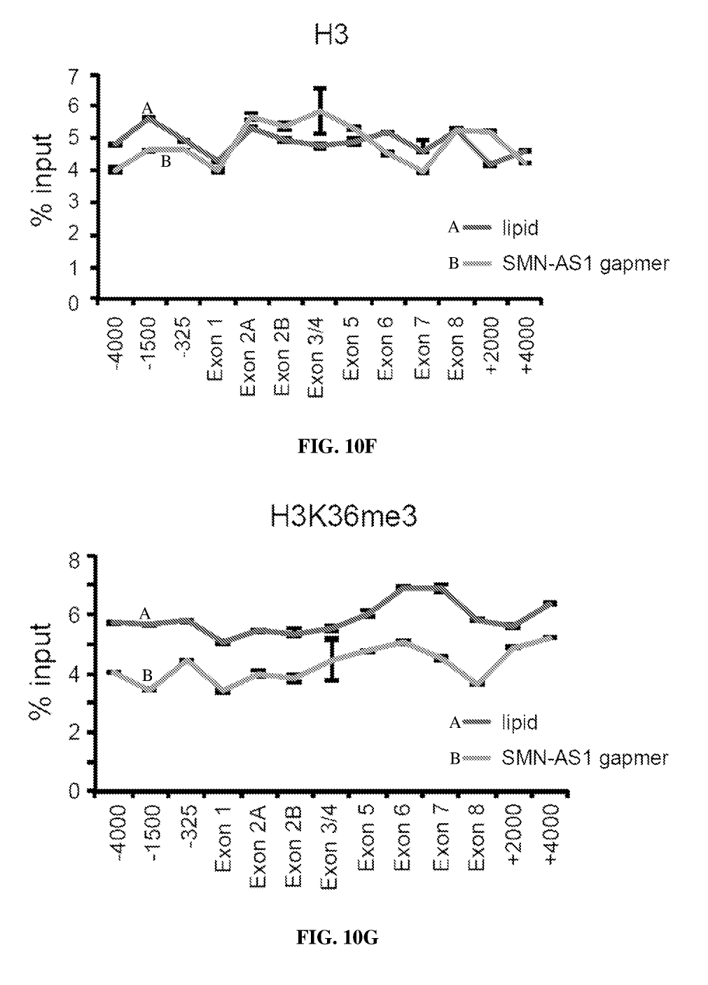

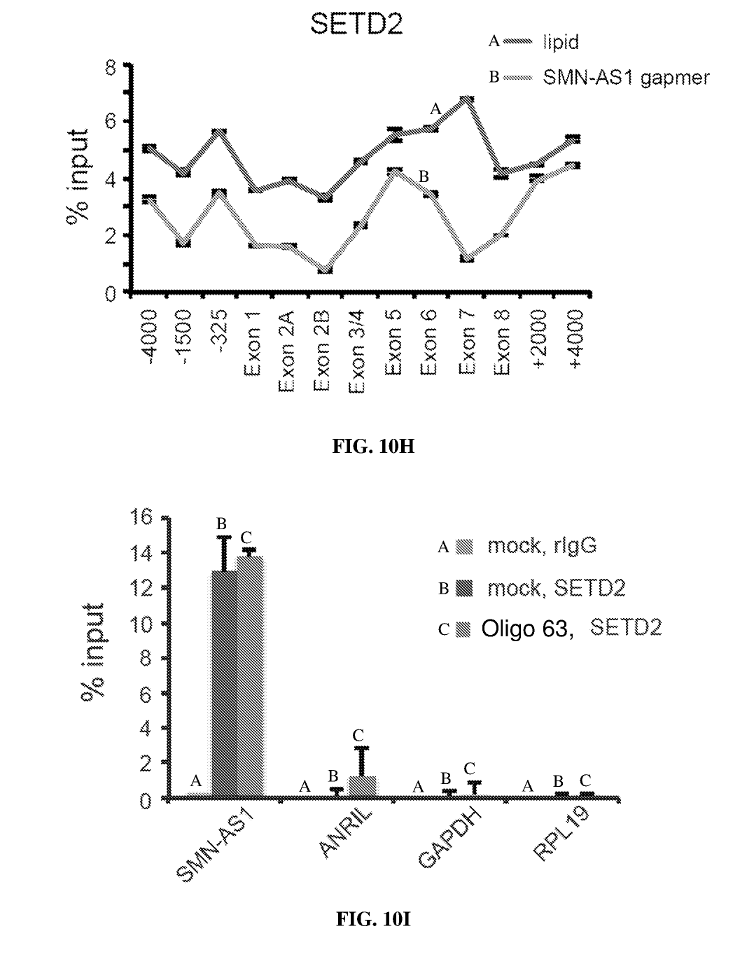

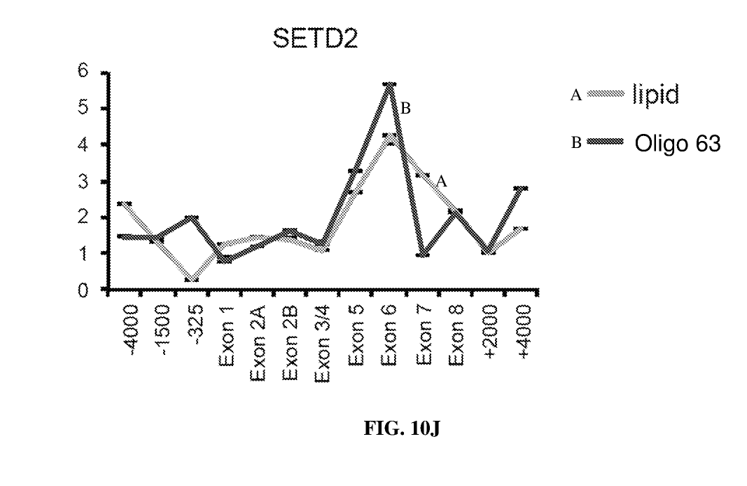

The data herein suggests that SMN-AS1 recruits PRC2 to suppress the expression of its protein-coding neighbor, SMN2. Next, it was examined as to whether RNaseH-mediated degradation of SMN-AS1 by a gapmer ASO would lead to SMN2 upregulation. Surprisingly, knocking down SMN-AS1 did not lead to an increase of SMN mRNA (FIGS. 10A-10C) despite the loss of PRC2 binding and H3K27me3 levels at the SMN2 locus as observed by ChIP (FIGS. 10D-10E). Interestingly, SMN-AS1 knockdown led to a decrease in H3K36me3 levels (FIG. 10G) and no increase in RNA Pol II, phosphor-Ser2. Next, the effect of a H3K36me3 methyltransferase SETD2, a chromatin modifier associated with active transcription, binding to the SMN2 locus was examined. Indeed, SMN-AS1 knockdown resulted in the loss of SETD2 association with the gene body. nRIP demonstrated that SETD2 binds to SMN-AS1 and that, unlike PRC2 binding, SETD2 association is unaffected by treatment with Oligo 63 (FIGS. 10I and 10J). These findings indicate that SMN-AS1 can bind two chromatin-modifying complexes with opposing activities, PRC2 and SETD2, but that only PRC2 is selectively blocked from associating with SMN-AS1 with Oligo 63. Taken together, these data show that degrading SMN-AS1 does not upregulate SMN expression in contrast to sterically blocking PRC2:SMN-AS1 interaction. Without wishing to be bound by theory, is thought that the SMN-AS1 functions as molecular scaffold that recruits both a transcriptional activator and a repressor to the SMN2 locus to fine-tune its expression. Therefore, selective disruption of PRC2 binding and its recruitment is necessary for SMN2 upregulation.

### Blocking PRC2 Recruitment Results in SMN2 Upregulation in Fibroblasts

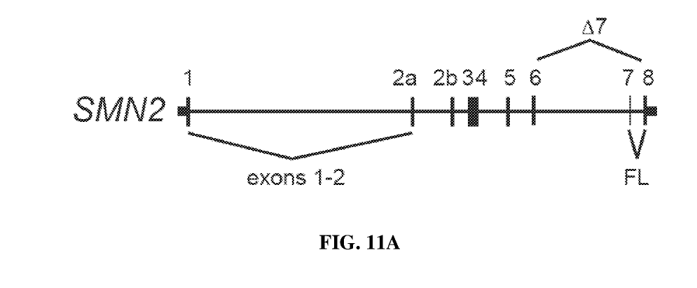

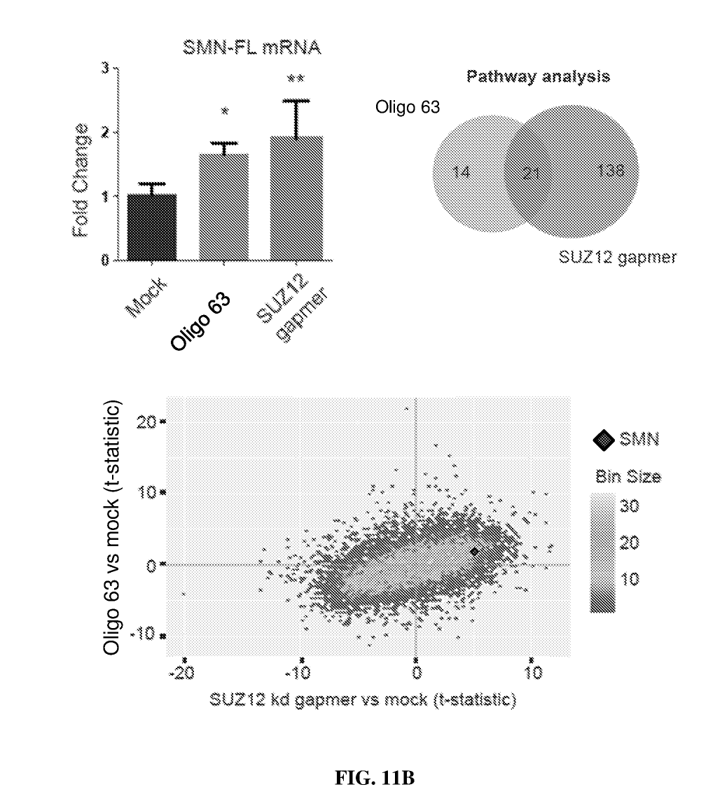

SMN2 mRNA upregulation resulting from the disruption of the PRC2:SMN-AS1 interaction and the subsequent epigenetic changes at the SMN locus were further characterized. The SMA fibroblast line GM09677, which carries two copies of the SMN2 gene and is homozygous for SMN1 exons 7 and 8 deletion, was used. Consistent with the transcriptional activation mechanism, RT-qPCR analyses with a few primer sets detect a concentration-dependent increase of various SMN mRNA transcripts, including all SMN isoforms (exon 1-2) as well as isoforms including or excluding exon 7, SMN-FL, and SMNΔ7, respectively (FIGS. 2C, 11A). In agreement with this, overall SMN protein levels also increased, as shown by ELISA after 5 days of treatment (FIG. 2D). Western blotting revealed that this increase could be attributed to the 38-kDa SMN protein (FIG. 2E). ELISA and Western analyses both indicated up to 4-fold protein upregulation following treatment in SMA fibroblasts. Taken together, blocking the interaction of PRC2 with its recruiting lncRNA resulted in upregulation of both SMN mRNA and protein.

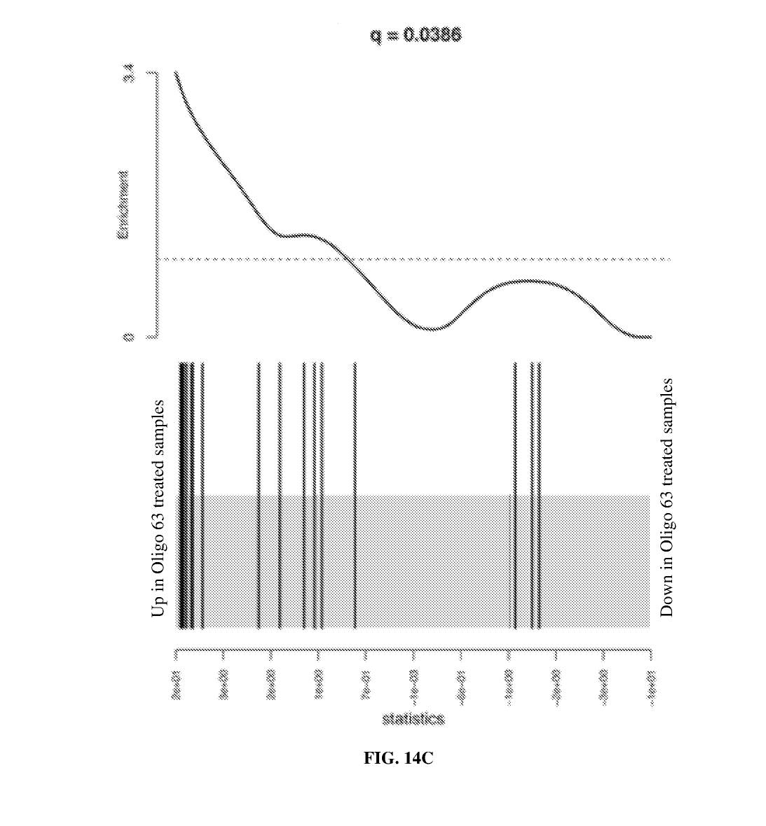

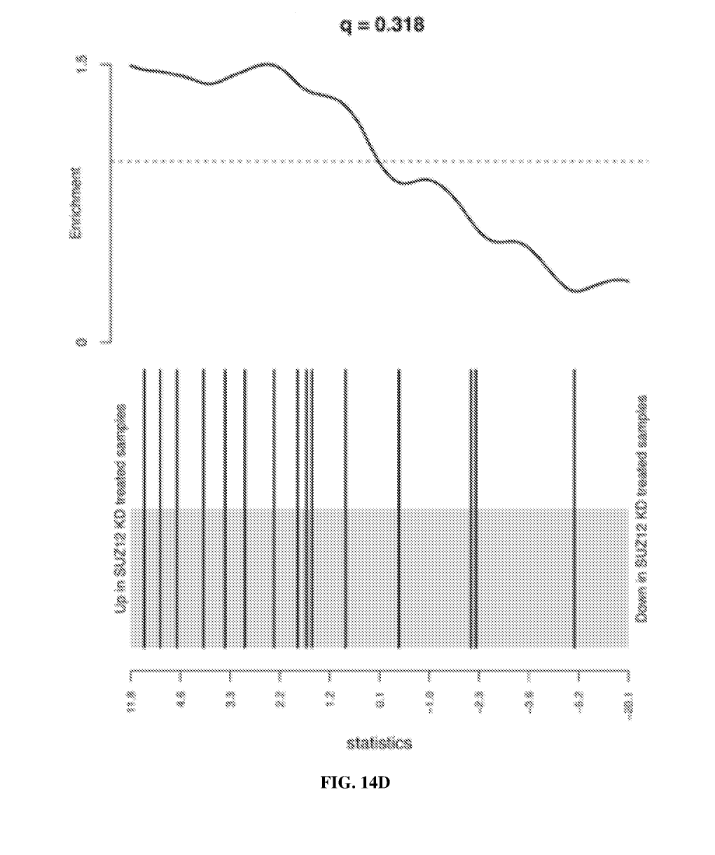

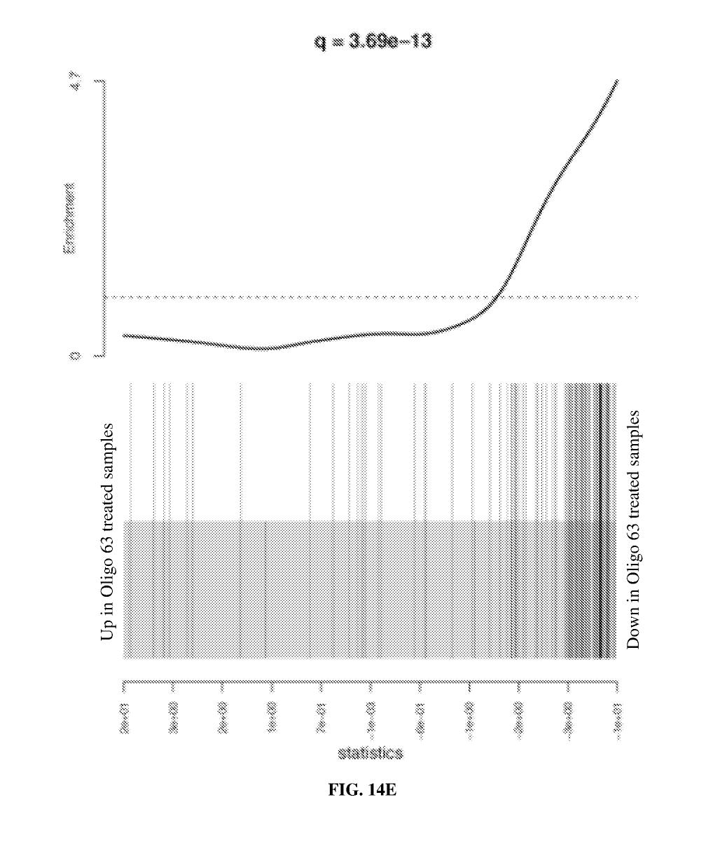

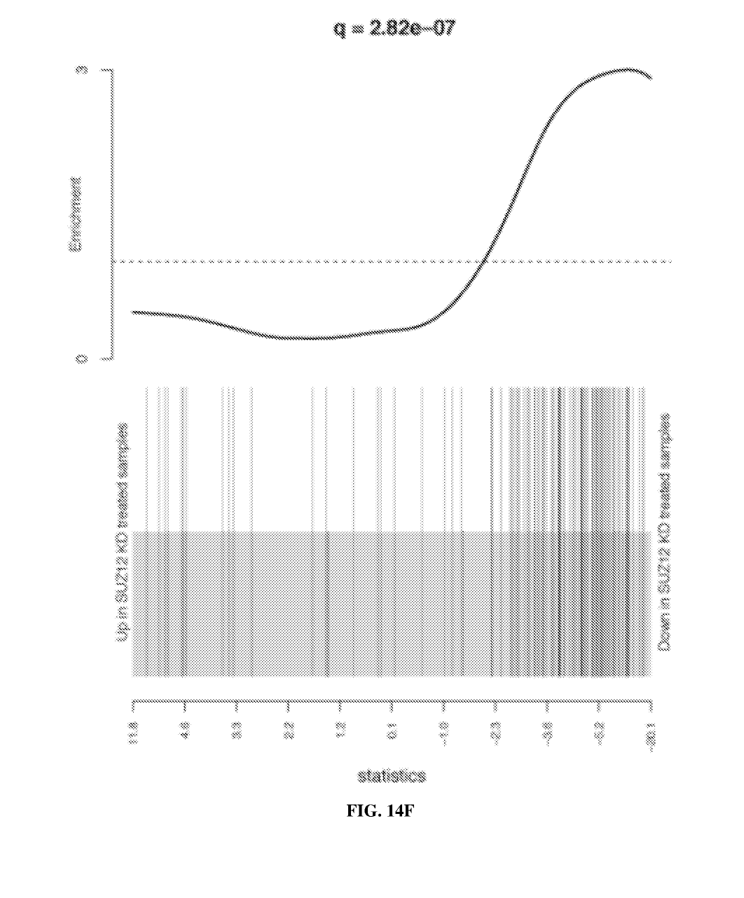

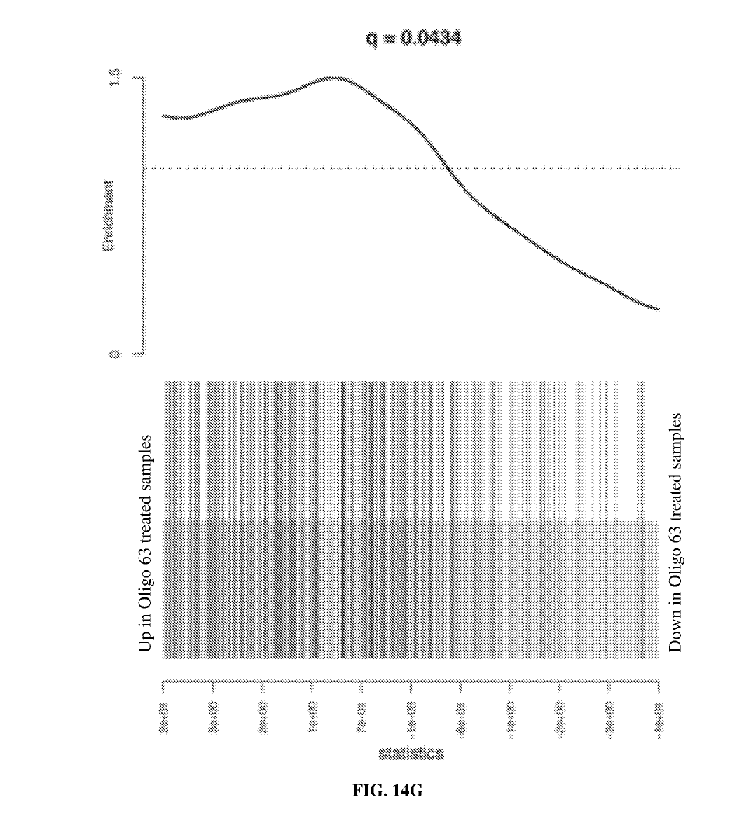

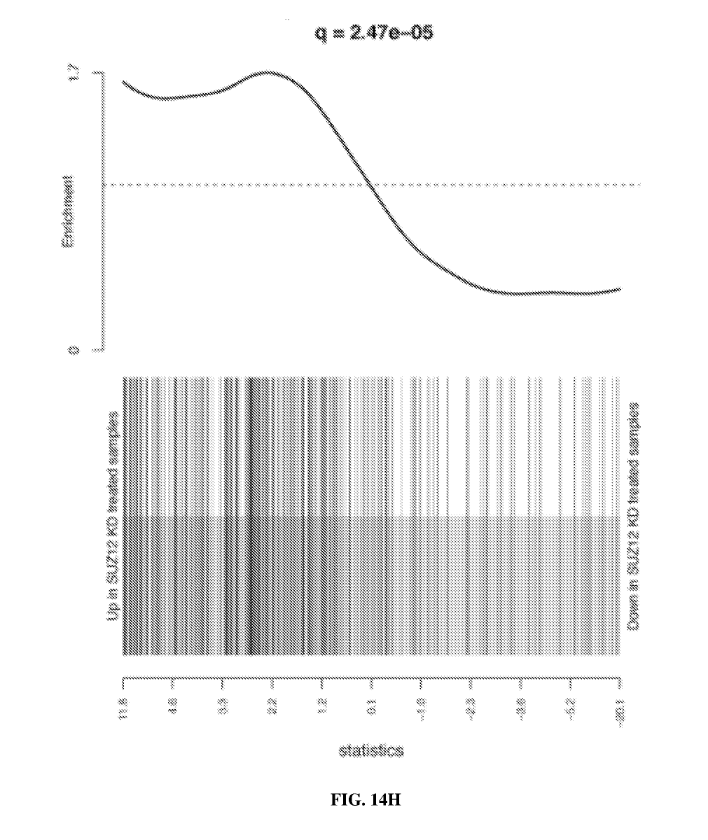

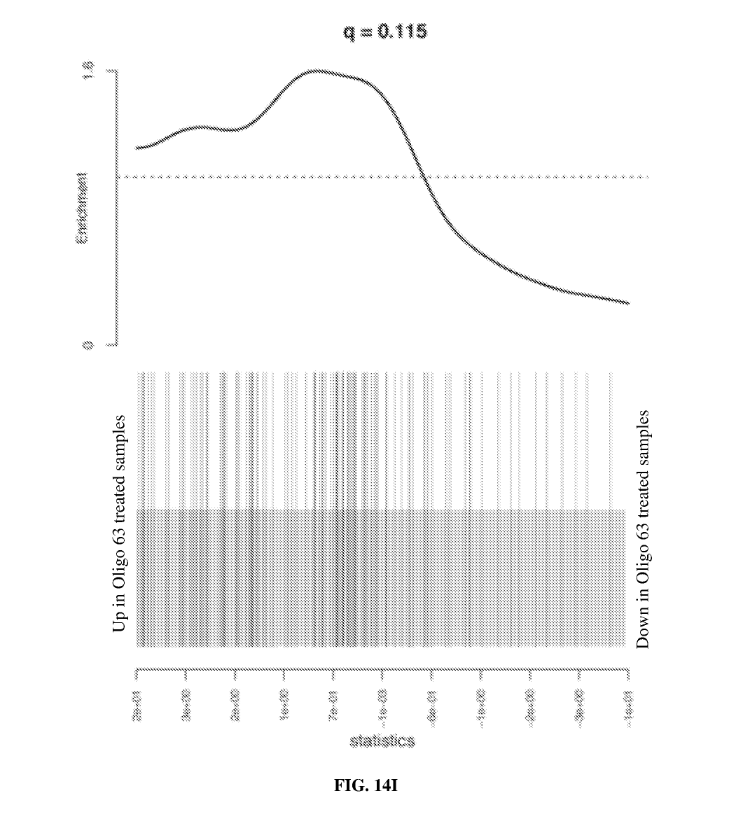

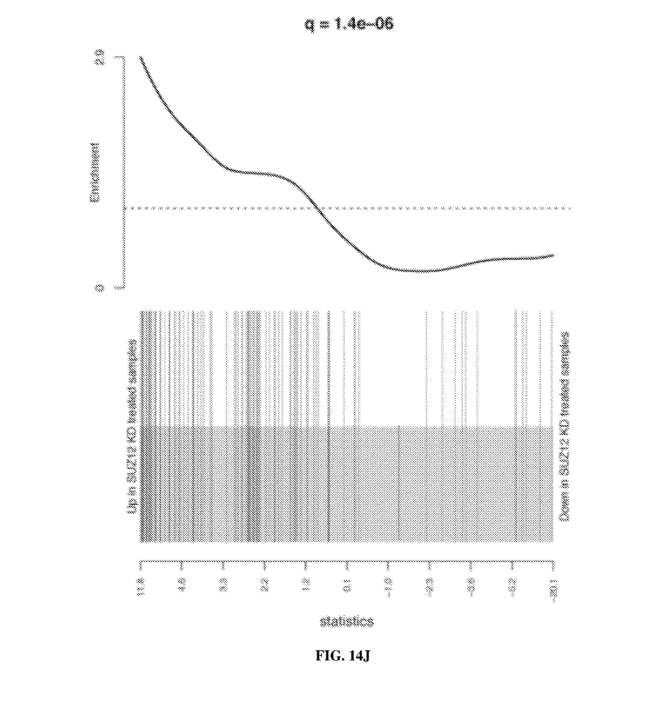

To determine how targeting the disruption of PRC2:SMN-AS1 interactions might affect PRC2 targets globally, RNA-sequencing was performed after transfection of the mixmer oligo, Oligo 63, or a gapmer ASO targeting SUZ12, a subunit of the PRC2 complex in SMA fibroblasts. Treatment with either the mixmer oligo or the SUZ12 gapmer ASO for 2 and 3 days resulted in significant increases in SMN mRNA levels compared to transfection control samples by RT-qPCR (FIG. 11B). Globally, there were approximately four-fold more gene expression changes with the SUZ12 gapmer ASO treatment than with Oligo 63 treatment that had at least a 1.5 fold change (q<0.05) as depicted by a scatterplot of the moderated t-statistics of the gene expression changes that occurred with oligo treatments. Focusing more locally by examining the nearest neighboring genes changing significantly in response to Oligo 63 treatment, the closest differentially expressed genes upstream (ADAMTS6) and downstream (BDP1) of SMN2 are 4.6 Mb and 1.4 Mb away, respectively. The nearest significant neighbor genes that changed after SUZ12 kd were TAF9, 0.8 Mb upstream, and BDP1, 1.4 Mb downstream, of SMN2. Pathway gene set analyses identified significant pathways (q<0.1) with each oligo treatment. While there was overlap between the oligo treatments, many more pathways changed separately with SUZ12 knockdown (FIG. 11B and FIG. 14A-J).

### Blocking PRC2 Recruitment Results in SMN2 Upregulation in Neuronal Cultures

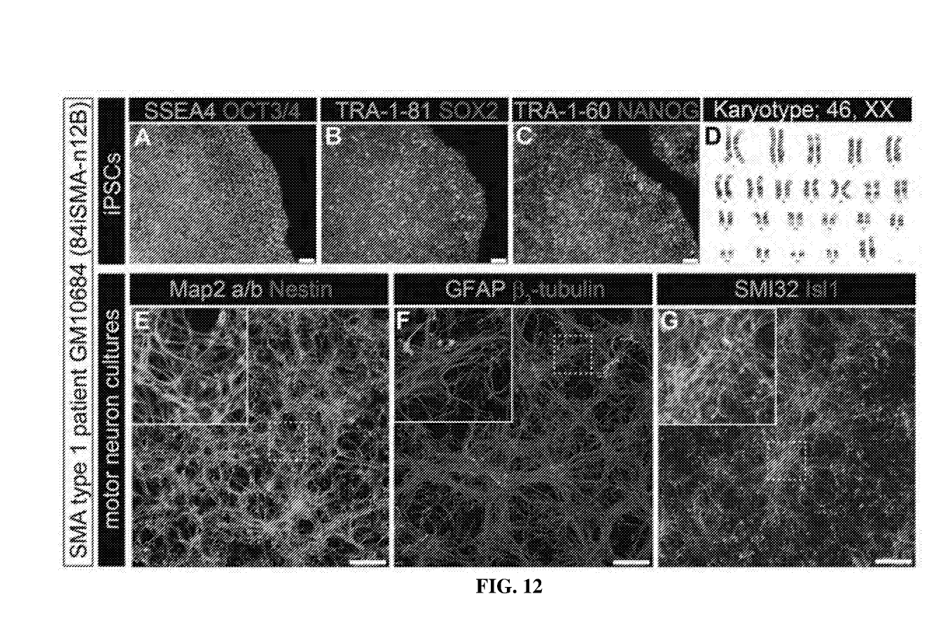

While SMN expression is ubiquitous, its expression is highest in the central nervous system (FIG. 1D) (Boda et al., 2004), particularly in spinal motor neurons where the disease is manifested (Battaglia et al., 1997; Monani, 2005; Burghes and Beattie, 2009). To assess the activity of Oligo 63 in disease-relevant cell types, SMN expression in two neuronal cell types was examined. First, induced pluripotent stem cells (iPSC) derived from SMA patient fibroblasts were generated and differentiated into SMI32+ motor neurons (FIG. 12). After treating with an activating ASO, SMN-FL mRNA increased 1.8-fold relative to untreated motor neurons (FIGS. 2G-2H). As expected, EZH2 knockdown also led to similar increase in SMN-FL mRNA. The delayed increase in human SMN-FL mRNA levels in neurons relative to fibroblasts may be partially due to the mode of delivery (unassisted delivery versus transfection) and/or the non-proliferating state of the neuronal cells versus the highly proliferative fibroblasts. Consistent with the latter, the rate of H3K27me3 removal from the chromatin of non-dividing cells is slower than in proliferating cells (Agger et al., 2007). Taken together, these data show that disrupting the PRC2:SMN-AS1 interaction leads to SMN upregulation in disease-relevant and post-mitotic neuronal cells.

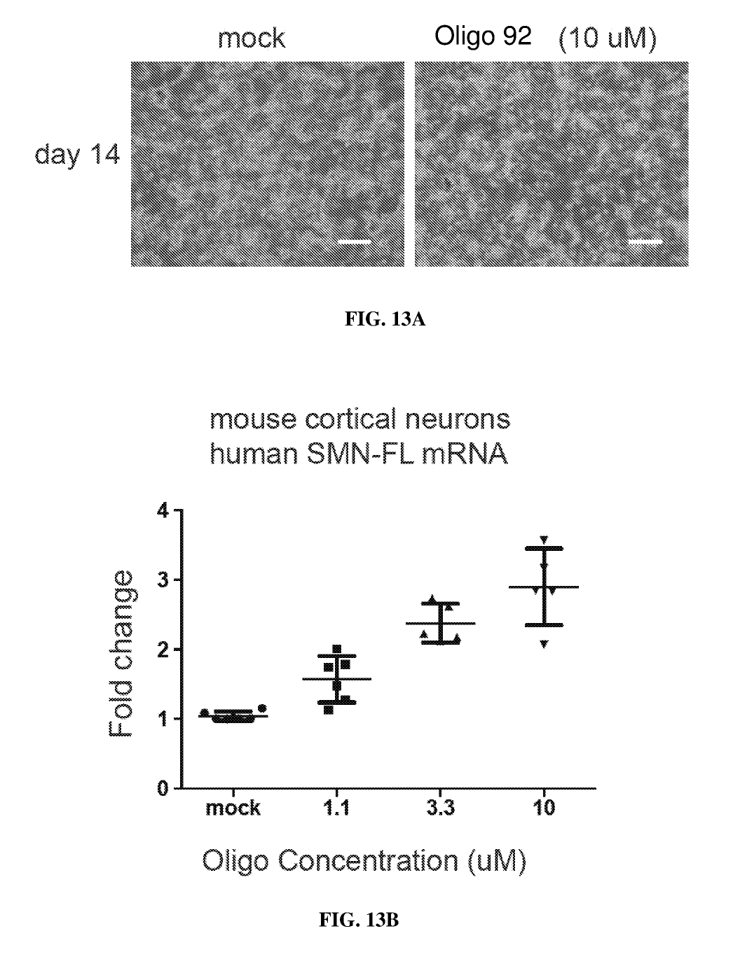

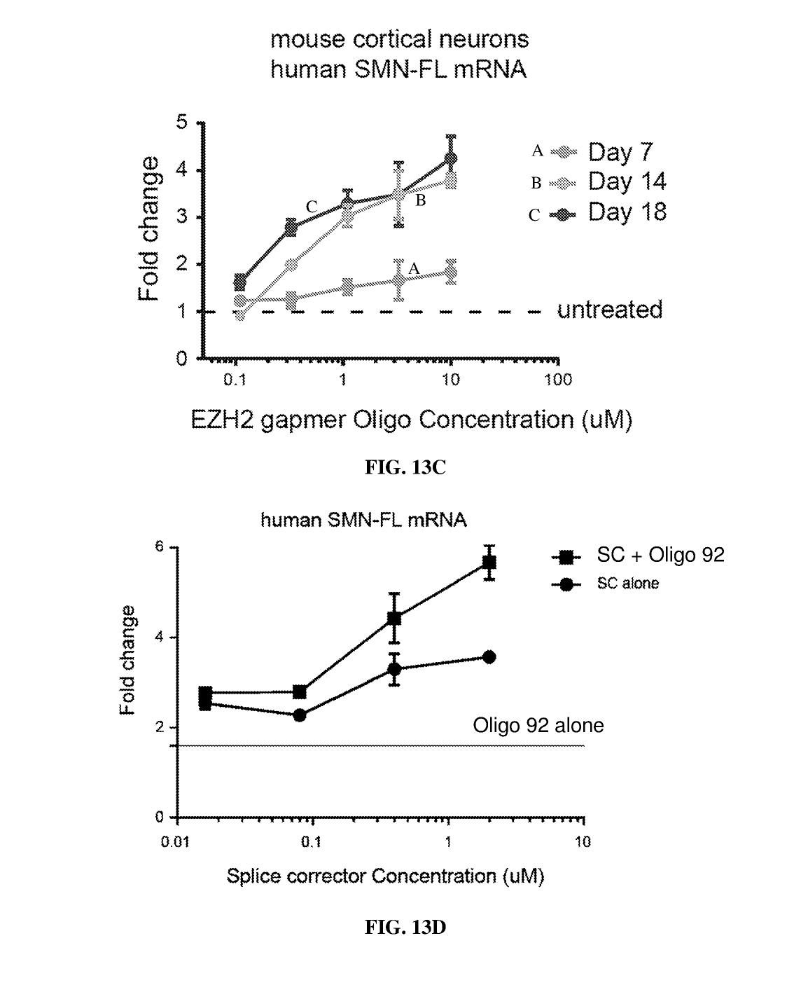

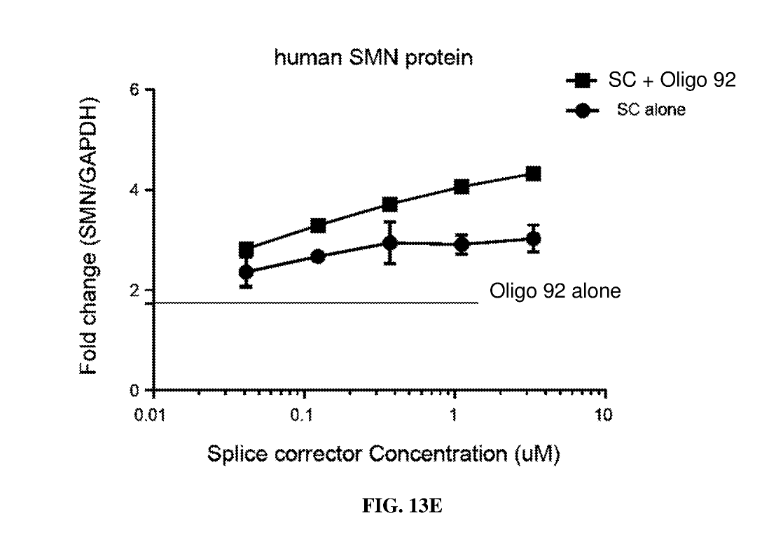

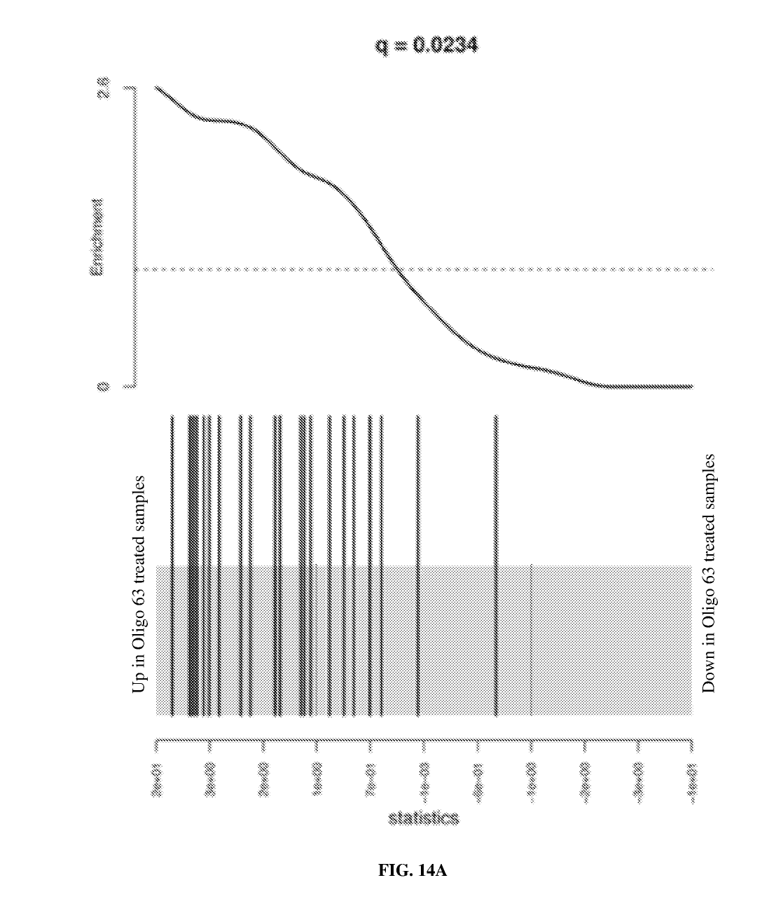

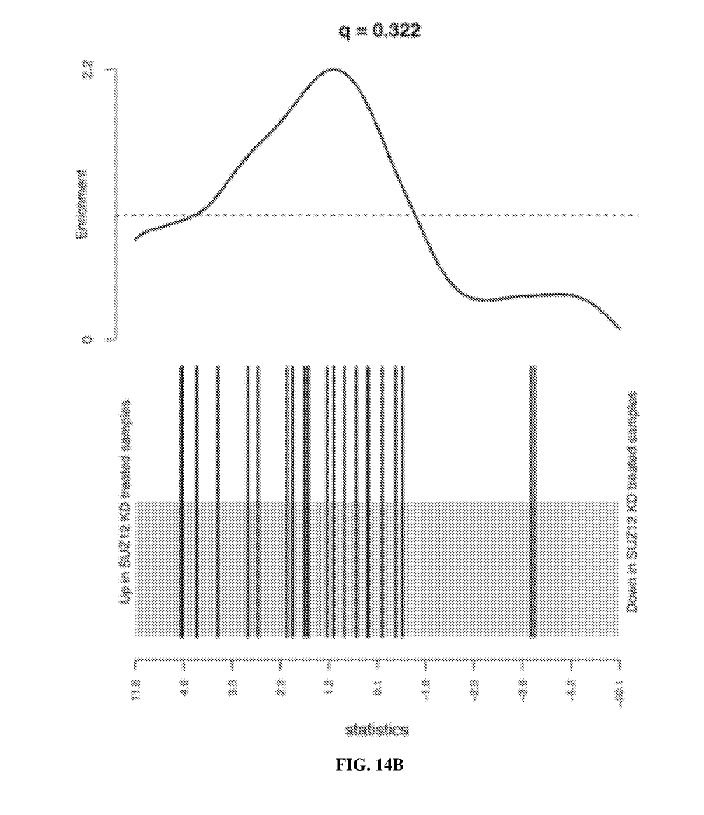

Primary cortical neuronal cells from E14 embryos of the 5025 SMA mice were also prepared and then treated with a chemical variant of Oligo 63, called Oligo 92, that targets the same SMN-AS1 sequence and may have a more favorable in vivo safety profile. Oligo 92 was added to culture medium at 1.1, 3.3, and 10 μM for 14 days without obvious toxicity or changes in cell morphology (FIG. 13A). A concentration-dependent increase in SMN-FL mRNA with a 3-fold increase at 10 μM following 14 days of treatment was observed (FIG. 13B). Consistent with the results obtained from patient fibroblasts (FIG. 7B), cortical neurons treated with an EZH2 gapmer ASO resulted in a concentration-dependent increase in SMN-FL mRNA levels (FIG. 13D). Several other unrelated ASOs were tested and changes in SMN-FL levels were not observed. The findings from ex vivo cortical neurons lend additional support to the transcriptional activation mechanism in terminally differentiated neuronal cells.

Combination of Transcriptional Upregulation and Splice Correcting Oligo Increases SMN-FL mRNA

Splice correcting modifiers are designed to facilitate the inclusion of exon 7 during splicing of SMN2 mRNA. Consequently, SMN-FL mRNA and functional SMN protein containing exon 7 would be produced. While steady-state total SMN mRNA levels would not increase with a splice correcting modifier, the shift to increase SMN-FL mRNA levels has been demonstrated to be beneficial to survival in mice (Hua et al., 2010; Palacino et al., 2015) and in humans (Chiriboga et al., 2016). Since the transcriptional activation approach upregulates SMN through a distinct mechanism from that of a splice corrector, it was thought that combining these two mechanisms would be more effective than either one of the two approaches alone. The 5025 cortical neurons were treated with either a splice correcting ASO (SCO), a transcriptional activating mixmer ASO (Oligo 92), or a combination of the two ASOs for 14 days to measure the levels of SMN-FL mRNA (FIG. 13D). While treatment with the SCO alone resulted in a 2-3-fold increase with the SCO, an additional 1.8-fold increase was observed in the presence of Oligo 92. This effect was also observed with increases in the human SMN protein levels by a human-specific ELISA (FIG. 13E). Whether the transcriptional activating mixmers affected mouse smn levels were previously tested and no changes were observed, as expected, because the transcriptional activating mixmer does not target any sequence within the mouse smn locus. While the SCO upregulated SMN-FL protein levels approximately 2.5-fold, the combination resulted in the increase of SMN levels to 4-fold. These data provide evidence that Oligo 92 increases SMN-FL mRNA and SMN protein levels by a mechanism that is independent and complementary to that of a SCO.

### Discussion

There is presently no approved disease-modifying therapeutic for SMA and treatments are focused on addressing symptoms ranging from respiratory complications to muscle atrophy. Currently, different approaches to treat SMA are being tested in clinical trials, most of which utilize splice correction mechanism to include exon 7 of SMN2 (reviewed by Cherry and Androphy, 2012). Distinct from the splice correction approach, a novel transcriptional upregulation method to selectively upregulate endogenous SMN mRNA and protein is reported. The overall changes in PRC2 and RNA Polymerase II occupancy, and histone modifications suggest that the increase in steady state levels of SMN2 arises at the transcriptional level. Indeed, when mouse primary cortical neurons were treated with the transcription activating ASO and a splice correcting oligo, an enhanced effect of SMN-FL mRNA and protein beyond that offered by a splice-correcting therapy alone was observed, which may potentially confer greater therapeutic benefit.

The discovery and characterization of SMN-AS1, which recruits both a transcriptional repressor and activator to a gene, suggests that lncRNAs may actively regulate and fine tune targeted gene expression. Indeed, localized H3K27me3 chromatin marks deposited by PRC2 along the FGFR2 gene block the inclusion of a particular exon in a cell type-specific manner. When the same region is marked by H3K36me3 (conferred by SETD2 in a different cell type), this alternative exon is included in the FGFR2 transcript (Luco et al., 2010). In the case of the SMN2 locus, knockdown of PRC2 did not promote alternative splicing to include exon 7, but instead led to overall increases in SMN transcripts. It is still being elucidated exactly why the association of SETD2 to SMN-AS1 appears to be required for full transcriptional activation of SMN2. However, in the absence of SMN-AS1 when SETD2 is no longer recruited and H3K36me3 levels remain unchanged, even the concomitant loss of PRC2 is insufficient to increase SMN2 mRNA levels. Taken together, these data suggest that chromatin changes, such as increased H3K36me3 levels, which are associated with productive transcriptional elongation, are important for full transcriptional activity at the SMN2 locus.

In summary, the gene upregulation technology described herein was shown to disrupt the interaction between PRC2 and a lncRNA. It was further shown that the specific blockade of PRC2:SMN-AS1 interaction is more efficacious than degrading SMN-AS1. This approach of preventing PRC2 recruitment to specific genomic locations potentially offers greater selectivity and elicits fewer unintended side effects than that of a small molecule EZH1/2 inhibitor. Notably, the degree of SMN upregulation is at a level that is considered to be therapeutic for SMA. With this proof-of-concept, it is thought that the upregulation platform could be applied to many other diseases in which a desirable gene is epigenetically silenced by a transcriptional repressive complex.

### Materials and Methods

Oligo Sequences.

The sequences of the oligos tested are shown in Table 1. All oligos in Table 1 are fully phosphorothioated with the exception of Oligo 69, which has the same base sequence as Oligo 92, but has a 50/50 mix of phosphorothioate and phosphodiester linkages.

TABLE 1
Oligo sequences

| | SEQ | | |
|---|---|---|---|
| Oligo | ID | Base | Full sequence with chemical |
| Name | NO | Sequence | modifications |
| Oligo | 6 | AGAUGCAG | lnaAs;omeGs;lnaAs;omeUs;lnaGs; |
| 52 | | TGCUCUT | omeCs;lnaAs;omeGs;lnaTs;omeGs; |
| | | | lnamCs;omeUs;lnamCs;omeUs;lnaT |
| Oligo | 7 | CATAGUGG | lnamCs;omeAs;lnaTs;omeAs;lnaGs; |
| 63 | | AACAGAT | omeUs;lnaGs;omeGs;lnaAs;omeAs; |
| | | | lnamCs;omeAs;lnaGs;omeAs;lnaT |
| Splice | 8 | TCACTTTC | moeTs;moemCs;moeAs;moemCs; |
| cor- | | ATAATGCT | moeTs;moeTs;moeTs;moemCs;moeAs; |
| rector | | GG | moeTs;moeAs;moeAs;moeTs;moeGs; |
| | | | moemCs;moeTs;moeGs;moeG |
| Oligo | 9 | CATAGTGG | lnamCs;lnaAs;lnaTs;lnaAs;lnaGs; |
| 92 | | AACAGAT | lnaTs;lnaGs;omeGs;lnaAs;lnaAs; |
| | | | lnamCs;omeAs;lnaGs;omeAs;lnaT |
| Oligo | 9 | CATAGTGG | lnamCs;lnaAs;lnaTs;lnaAs;lnaGo; |
| 69 | | AACAGAT | lnaTo;lnaGo;omeGo;lnaAo;lnaAo; |
| | | | lnamCo;omeAs;lnaGs;omeAs;lnaT |

Key:
lna = Locked Nucleic Acid (LNA);
lnamC = LNA 5′ methyl cytosine;
ome = 2′-O-methyl;
moe = 2′-O-(2-methoxyethyl);
moemC = 2′-O-(2-methoxyethyl) 5′ methyl cytosine;
s = phosphorothioate linkage;
o = phosphodiester linkage.

RNA Sequencing.

RNA from GM09677 fibroblasts that were transfected with Oligo 63, SUZ12 gapmer ASO, and lipid controls. were sequenced (300 bp paired-end) on the NextSeq500 using Illumina TruScq stranded total RNA-scq library preparation kits.

Northern Blots.

RNA preparation: Total RNA from human fetal brain and lung tissue was obtained from ClonTech and treated with RiboMinus (Life Technologies). 500 ng of rRNA-depleted RNA was fractionated on a 1% agarose gel in 1×MOPS buffer. RNA was capillary transferred to BrightStar Plus nylon membrane (Ambion) overnight in 20×SSC buffer, then crosslinked by UV exposure. For mouse Northern blots, RNA was isolated from 5025 WT brain tissue and WT brain tissue, and treated with RiboMinus as above. Approximately 750 ng RNA was loaded per lane.

Probe Preparation.

DNA templates containing a T7 promoter for in vitro synthesis of radiolabeled RNA probes were generated by PCR from a human fetal brain cDNA library or mouse brain cDNA library with primer pairs listed in the Table 2 or SMN-FL (Hua et al., 2010).

TABLE 2
Probes and Primers

| | | SEQ |
|---|---|---|
| | Sequence | ID NO |
| Probe or Primer Name | | |
| PRC2 binding region | TAATACGACTCACTATAGTCCCCTAAACAAAGACG | 10 |
| T7 Forward | AGGTC | |
| PRC2 binding region | ATACTGTGTATTGGGATGGGGT | 11 |
| Reverse | | |
| non binding region T7 | TAATACGACTCACTATAGAAAATCAGCCCCCTGAG | 12 |
| Forward | ACCAA | |
| non binding region | TTTTCGAGATGGAGTCTTGCTCTG | 13 |
| Reverse | | |
| RepA I-IV T7 Forward | TAATACGACTCACTATAGATTGTTTATATATTCTTG | 14 |
| | CCCATCGGGG | |
| RepA I-IV Reverse | CACAAAACCATATTTCCATCCACCAAGC | 15 |
| MBP T7 Forward | TAATACGACTCACTATAGATGAAAATAAAAACAG | 16 |
| | GTGCAC | |
| MBP Reverse | CAGATCTTTGTTATAAATCAGCGATAACG | 17 |
| RT-PCR primer or | | |
| probe name | | |
| SMN-AS1 F (set 1) | GCAGTGCTCTTGTAGTCCCA | 18 |
| SMN-AS1 R (set 1) | CCTCCTTATGGCATAGACACC | 19 |
| SMN-AS1 probe (set 1) | CTTCTGCCAGGAAAGAAGGCAACC | 20 |
| SMN2-FL forward primer | GCTGATGCTTTGGGAAGTATGTTA | 21 |
| SMN2-FL reverse primer | CACCTTCCTTCTTTTTGATTTTGTC | 22 |
| SMN2-FL probe | TACATGAGTGGCTATCATACT | 23 |
| Δ7 SMN2 forward primer | TGGACCACCAATAATTCCCC | 24 |
| Δ7 SMN2 reverse primer | ATGCCAGCATTTCCATATAATAGCC | 25 |
| Δ7 SMN2 probe | TCCAGATTCTCTTGATGATG | 26 |
| ChIP Primer name | | |
| Exon 2B Forward | CATTTGTGAAACTTCGGGTAAACCA | 27 |
| Exon 2B Reverse | GTAAGGAAGCTGCAGTATTCTTCTTTTG | 28 |
| Exon 2B Probe | CACACCTAAAAGAAAACC | 29 |
| Exon 3/4 Forward | TTTACCCAGCTACCATTGCTTCAA | 30 |
| Exon 3/4 Reverse | CGGACAGATTTTGCTCCTCTCTATT | 31 |
| Exon 3/4 Probe | ACCTGTGTTGTGGTTTAC | 32 |
| Exon 5 Forward | CCTTCTGGACCACCAGTAAGTAAAAA | 33 |
| Exon 5 Reverse | GGGATGTTCTACAATGACATTTTACAATCC | 34 |
| Exon 5 Probe | TTGCTTTCACATACAATTTG | 35 |
| Exon 6 Forward | ATCACTCAGCATCTTTTCCTGACAA | 36 |
| Exon 6 Reverse | GCCTCAGACAGTTGTATTTTTTTATTTTTATTTTTTA | 37 |
| | GTAATATA | |
| Exon 6 Probe | ATGTGACTTTGTTTTGTAAATTTA | 38 |
| Exon 7 Forward | AAAATGTCTTGTGAAACAAAATGCTTTTTA | 39 |
| Exon 7 Reverse | CCTTCTTTTTGATTTTGTCTGAAACCTGTA | 40 |
| Exon 7 Probe | AAAATAAAGGAAGTTAAAAAAAATAG | 41 |
| Exon 8 Forward | CGGTGGTGAGGCAGTTGA | 42 |
| Exon 8 Reverse | CCCTTCTCACAGCTCATAAAATTACCAATAAT | 43 |
| Exon 8 Probe | AATCCACATTCAAATTTTC | 44 |
| Down Forward | TCCCATTTTGTAGGTTGCCTGTT | 45 |
| Down Reverse | ACTAAAGAGCTTCTGCACAGCAAA | 46 |
| Down Probe | CACTCTGATGGTAGTTTCT | 47 |
| Down2 Forward | CAGCCTCCTGAGTAGCTAGGATTA | 48 |
| Down2 Reverse | GGTGAAACCCCGTCTCTACTAAAAAA | 49 |
| Down2 Probe | CAGGCACACGCCACCAT | 50 |
| HOXC13prom Forward | GAGACTTCAGCAGTCACAGTGAT | 51 |
| HOXC13prom Reverse | GGAGGAGAGCGCTGTAACT | 52 |
| HOXC13prom Probe | TCCGGTGCACATCCTA | 53 |

Cell Culture for RNA-FISH.

GM09677 Human Eye Lens Fibroblast (Coriell) adherent cells were grown in Eagle's Minimum Essential Medium (EMEM) (ATCC) in a humidified 37° C. incubator at 5% CO₂. F-12K and EMEM media were supplemented with 10% FBS (Fisher Product number SH30071.03), 5 mL of Pen/Strep (Life technologies). F-12 was further supplemented with Normocin (InvivoGen). Cells were grown on 12 mm microscope circular cover glass No. 1 (Fisher #12-545-80) in 24 well flat bottom cell culture plates (E&K). Stellaris® RNA FISH

Probe sets were designed against genomic regions listed in Table 2. They were labeled with Quasar 570® (SMN1/2 exons), Quasar 670 (SMN1/2 introns), and Cal Fluor® Red 610 (SMN1/2-AS1). Stellaris RNA fluorescence in situ hybridization (FISH) was performed as described in the Alternative Protocol for Adherent Cells (UI-207267 Rev. 1.0) with the following modifications: 12 mm diameter coverslips were used. 25 μL hybridization solution was used with a final concentration of each probe set of 250 nM. The wash buffer volumes were halved. The FITC, Cy3, Cy3.5, and Cy5.5 channels were used to capture the signals from each probe set and the FITC channel was used to identify cellular autofluorescence. The filter sets from Chroma were: 49001-ET-FITC, SP102v1-Cy3, SP103v2-Cy3.5, and 41023-Cy5.5. The exposure times were 1 sec for FITC, Quasar 570, and Cal Fluor Red 610, and 2 sec for Quasar 670.

Oligonucleotide Transfection for FISH.

SMA fibroblasts were transfected at 70% confluence by using oligonucleotides complexed with Invitrogen Lipofectamine 3000 (Pub Part #100022234, Pub # MAN0009872, Rev. B. O), and fixed after two days. 2 ng DNA and 4 μL P3000 reagent was used per 50 μL of DNA master mix was. 0.375 μL Lipofectamine 3000 reagent was used per 25 μL of Opti-MEM.

RT-qPCR.

Total RNA from 20 human tissues (Clontech) were used for cDNA synthesis using High Capacity cDNA Reverse Transcription Kit (Applied Biosystems). RT-qPCR SMN-AS1 levels data were normalized to levels from adrenal gland. GM09677 fibroblasts were plated a 24-well tissue culture plate at 4×10⁴ cells/well in MEM containing 10% FBS and 1× non-essential amino acids. Fibroblasts were treated with ASOs the following day. After 2 days cells were lysed and mRNA was purified using E-Z 96 Total RNA Kit (Omega Bio-Tek). SMA iPS-derived motor neurons were lysed with TRIzol for RNA isolation according to the manufacturer's protocol. RNA from mouse cortical neurons was extracted using the RNeasy kit (Qiagen) according to the manufacturers protocol. All cDNAs were synthesized using High Capacity cDNA Reverse Transcription Kit (Applied Biosystems). SMN FL, SMN Δ7, and SMN Exon 1-2, and GUSB mRNA expression was quantified by predesigned TaqMan real-time PCR assays. A list of custom-designed real-time PCR assays is listed in Table 2.

Oligonucleotide Transfection for ChIP.

SMA fibroblasts were transfected at 70% confluence by using oligonucleotides complexed with Lipofectamine 2000 (Invitrogen) following the protocol suggested by the manufacturer in the 96-well and 24-well format. For ChIP, cells were transfected in 15 cm plate and were transfected at 30 nM with Lipofectamine 2000 at a final volume of 20 mL. Cells were harvested 3 days post transfection.

RNA Immunoprecipitation.

RIP was performed using the Magna RIP RNA-Binding Protein Immunoprecipitation Kit (EMD Millipore) using a ChIP-grade anti-SUZ12 (Abcam), anti-EZH2 (Abcam), and anti-SETD2 (USBiological Life Sciences) antibodies. RNA was extracted with Trizol (Life Technologies) and transcribed to cDNA using the High Capacity cDNA Reverse Transcription Kit (Applied Biosystems). qPCR was performed on a StepOnePlus Real Time PCR System (Applied Biosystems) using Taqman Fast Advanced Mastermix (Applied Biosystems).

Electrophoretic Mobility Shift Assay.

DNA templates for EMSA probes containing T7 promoter sequences were generated by PCR using Phusion High Fidelity DNA Polymerase (NEB) and the specific primer sequences are listed in the Table 2. EMSAs were performed as described previously (Cifuentes-Rojas et al., 2014). Briefly, RNA probes were transcribed using the AmpliScribe T7 Flash Transcription Kit (Epicentre) and PAGE purified from 6% TBE urea gel. RNA probes were then dephosphorylated by calf intestinal alkaline phosphatase (NEB), purified by phenol-chloroform extraction, 5′ end-labeled with T4 Polynucleotide Kinase (NEB) and [γ-³²P]ATP (Perkin-Elmer), and purified with Illustra MicroSpin G-50 columns (GE Life Sciences). RNA probes were folded in 10 mM Tris pH 8.0, 1 mM EDTA, 300 mM NaCl by heating to 95° C., followed by incubations at 37° C. and at room temperature for 10 min each. MgCl2 and Hepes pH 7.5 were then added to 10 mM each and probes were put on ice. 1 μl of 2,000 cpm/ml (2 nM final concentration) folded RNA was mixed with PRC2 (EZH2/SUZ12/EED; BPS Bioscience) at the indicated concentration and 50 ng/ml yeast tRNA (Ambion) in 20 μl final concentration of binding buffer (50 mM Tris-HCl pH 8.0, 100 mM NaCl, 5 mM MgCl2, 10 mg/ml BSA, 0.05% NP40, 1 mM DTT, 20 U RNaseOUT [Invitrogen], and 5% glycerol). Binding reactions were incubated for 20 min at 30° C. and applied on a 0.4% hyper-strength agarose (Sigma) gel in THEM buffer (66 mM HEPES, 34 mM Tris, 0.1 mM disodium EDTA, and 10 mM MgCl₂). Gels were run for 1 hr at 130 V with buffer recirculation at 4° C., dried and exposed to a phosphorimager screen. Screens were scanned in a Storm 860 phosphorimager (Molecular Dynamics), data were quantified by Quantity One and normalized as described (Wong and Lohman, 1993). K_{D}s were calculated with Graphpad Prism by fitting the data to a one-site specific binding model.

Western Blot.

Five days post-transfection cells were lysed using the extraction buffer from the SMN ELISA kit (Enzo) with Protease inhibitor cocktail tablets (Roche). The total protein content was determined with the total BCA assay (Promega). Samples and Hi Mark prestained ladder (Invitrogen) were then run on the Bis-tris gel and the protein was transferred to nitrocellulose membrane. Non-specific binding was blocked using blocking buffer from Licor overnight at 4° C. SMN antibody (BD Catalog #610646) and Alpha tubulin antibody (abcam catalog # ab125267) and secondary anti-mouse and anti-rabbit −800 (Licor) were used and the blot was read using a LICOR-odyssey. Band intensities for SMN-FL protein and α-tubulin were quantified using Image Studio software.

ELISA Protocol.

GM09677 fibroblasts were plated a 24-well tissue culture plate at 4×10⁴ cells/well in MEM containing 10% FBS and 1× non-essential amino acids. Fibroblasts were treated with oligonucleotides the following day. After 5 days, cells were lysed and protein was quantified with the SMN ELISA Kit (Enzo Life Sciences, Inc.) and normalized to total protein content as determined by Micro BCA Protein Assay Kit (Thermo Scientific). For the human-specific ELISA used with the cortical neurons, a similar protocol was used. Briefly, cells were washed in cold PBS and lysed in RIPA buffer supplemented with protease inhibitor complete Tablets, mini EDTA—free EAS Ypack (Roche). Lysates were quantified by BCA and approximately 20-30 μg were used. A mouse monoclonal anti-SMN antibody was captured on high binding plates (Pierce) at 1 μg/mL; after blocking with BSA in PBS-0.05% Tween-20, lysates were incubated for 2 hours at RT; a rabbit polyclonal human SMN-specific antibody at 1 μg/mL was used for detection, followed by HRP-goat anti-rabbit (Invitrogen). The signal was measured with SuperSignal ELISA PICO chemiluminescent substrate (Thermo). Total GAPDH in the lysates was also quantified by ELISA (R&D Systems); SMN protein concentration was normalized to total GAPDH content.

Cortical Neuron Isolation.

Brains were isolated from E14 SMNΔ7 embryos and the cortex was dissected with the MACS neuronal tissue dissociation kit (Miltenyi Biotec). The collected cortical neurons were plated at 0.5×10⁶ cells per well in Neurobasal media (ThermoFisher), B-27 supplement (Thermofisher), and GlutaMax (ThermoFisher) in a 24-well plate coated with poly-D-lysine (Fisher). Cells were incubated at 37° C., 5% CO₂ for 4 days, allowing the cells to mature and networks to form before unassisted delivery of Oligo 63. After 14 days, the cells were harvested for RNA isolation.

### iPS Cell Culturing and Motor Neuron Differentiation Protocol.

SMA patient and control subject dermal fibroblasts or lymphoblastoid cell lines (LCLs) were obtained from the Coriell Institute for Medical Research. The iPSCs were grown to near confluence under normal maintenance conditions before the start of the differentiation as per protocols described previously (PMID: 25298370). Briefly, IPSCs were gently lifted by Accutase treatment for 5 min at 37° C. 1.5-2.5×10⁴ cells were subsequently placed in each well of a 384 well plate in defined neural differentiation medium with dual-SMAD inhibition (PMID:19252484). After 2 days, neural aggregates were transferred to low adherence flasks. Subsequently, neural aggregates were plated onto laminin-coated 6-well plates to induce rosette formation in media supplemented with 0.1 μM retinoic acid and 1 μM puromorphine along with 20 ng/ml BDNF, 200 ng/ml ascorbic acid, 20 ng/ml GDNF and 1 mM dbcAMP. Neural rosettes were isolated and the purified rosettes were subsequently supplemented with 100 ng/mL of EGF and FGF. These neural aggregates, termed iPSC-derived motor neuron precursor spheres (iMPS), were expanded over a 5 week period. For terminal differentiation, iMPS were disassociated with accutase and then plated onto laminin-coated plates over a 21 day period prior to harvest using the MN maturation media consisting of Neurobasal supplemented with 1% N₂, ascorbic acid (200 ng/ml), dibutyryl cyclic adenosine monophosphate BDNF (10 ng/ml), and GDNF (10 ng/ml). Oligo 63 treatments were carried out during this terminal differentiation period. Antibodies used for immunocytochemistry were as follows: SSEA4 and SOX2 (Millipore); TRA-1-60, TRA-1-81, OCT4, NANOG (Stemgent); TuJ1 (β3-tubulin) and Map2 a/b (Sigma); ISLET1 (R&D Systems); and SMI32 (Covance).

Chromatin Immunoprecipitation.

Cells were crosslinked with 1% formaldehyde for 10 minutes at room temperature and then quenched with glycine. Chromatin was prepared and sonicated (Covaris 5200) to a size range of 300-500 bp. Antibodies for H3, H3K27me3, H3K36me3, EZH2, and RNA Polymerase II Serine 2 (Abcam) and H3K4me3 (Millipore) were coupled to Protein G magnetic beads (NEB), washed, and then resuspended in IP blocking buffer. Chromatin lysates were added to the beads and immunoprecipitated overnight at 4° C. Antibodies against H3, H3K36me3, RNA Polymerase II phosphoserine 2, H3K27me3, and EZH2 were obtained from Abcam and the H3K4m3 antibody was obtained from Millipore. 10 ug of antibody was used per IP. IPs were washed, RNase A-treated (Roche), Proteinase K-treated (Roche), and then the crosslinks were reversed by incubation overnight at 65° C. DNA was purified, precipitated, and resuspended in nuclease-free water. Custom Taqman probe sets were used to deter nine DNA enrichment. Probes were designed using the custom design tool on the Life Technologies website. Primer sequences are listed in Table 2.

Bioinformatics Methods.

Mock, Oligo 63, and the SUZ12-KD gapmer treated GM09677 SMA fibroblasts were sequenced (151 bp paired-end) on an Illumina NextSeq 500 machine using the Illumina TruSeq polyA stranded RNAseq library preparation kits. For each of the treatments, there were two time points with each time point having two replicates for a total number of 4 samples per condition. FastQC (Andrews S. (2010). FastQC: a quality control tool for high throughput sequence data. Available online at: bioinformatics.babraham.ac.uk/projects/fastqc) was used to examine fastq quality metrics. Adapter and low quality sequences were trimmed from the reads using Trimmomatic (version 0.35) [PubMed ID (PMID): 24695404] with the following modules and settings: Crop to paired end length of 150 bp; IlluminaClip allowing for 2 seed mismatches, paired end seed score of 30, single end seed score of 10, minimum adapter length of 2, and while keeping both reads; SlidingWindow with a window size of 10 bp and sliding window minimum average phred score of 15; and finally reads were discarded if their length went below 36 base pairs. Next, rRNA reads were removed after aligning against rRNA sequences with bowtie2 v. 2.1.0 [PMID: 22388286].

The rRNA-depleted RNAseq fastq files were aligned with the STAR aligner (version 2.5.1a) [PMID: 23104886] to a modified version of hg38 Homo sapiens reference genome with a chromosomal segment duplication containing SMN2 (chr5:69,924,952-70,129,737) masked in order to align all SMN mapping reads to SMN1 and avoid multimapping. HTseq-count version 0.6.1 [PMID: 25260700] counted reads that overlapped gene features in the Gencode v24 gene annotation [PMID: 22955987 and PMID: 16925838].

Read counts were imported into R version 3.2.3 (R Core Team (2015). R: A language and environment for statistical computing. R Foundation for Statistical Computing, Vienna, Austria. R-project.org/) and were analyzed using Bioconductor [PMID: 25633503]. Lowly expressed genes across the samples were filtered using a mixture model from the SCAN.UPC R package version 2.12.1 [PMID: 24128763]. The remaining feature counts were scaled with TMM normalization [PMID: 20196867] and voom transformed [PMID: 24485249]. Limma version 3.26.7 [PMID: 25605792] was used to fit a linear model blocking time and looking at each oligo treatment vs. mock to identify significant changes in differential expression at an FDR (Benjamini Y., Hochberg Y. Controlling the false discovery rate: a practical and powerful approach to multiple testing. J Roy Statist Soc Ser B (Methodological) 1995; 57:289-300.) corrected p value <0.05 with a FC >1.5. Scatter plots were generated using the ggplot2R package 9H. Wickham. ggplot2: elegant graphics for data analysis. Springer New York, 2009.).

Pathway gene sets were obtained from the canonical pathway (C2) collection in the Molecular Signatures Database (MSigDB v5.0) [PMID: 16199517]. Significant pathways were identified using the competitive gene set testing method Camera with inter gene correlation set to 0.01 and with the same design matrix that was used in the differential expression analysis [PMID: 22638577]. A pathway was considered significant if it met a q value threshold <0.10. Barcode plots of the specific pathways were created using the barcodeplot function. Lastly, overrepresentation of differentially expressed genes or pathways between the different oligonucleotide treatments was evaluated with the hypergeometric test.

### REFERENCES

Agger, K., Cloos, P. A., Christensen, J., Pasini, D., Rose, S., Rappsilber, J., Issaeva, I., Canaani, E., Salcini, A. E., and Helin, K. (2007). UTX and JMJD3 are histone H3K27 demethylases involved in HOX gene regulation and development. Nature 449, 731-734.
Basu, A., Dasari, V., Mishra, R. K., and Khosla, S. (2014). The CpG island encompassing the promoter and first exon of human DNMT3L gene is a PcG/TrX response element (PRE). PLoS One 9, e93561.
Battaglia, G., Princivalle, A., Forti, F., Lizier, C., and Zeviani, M. (1997). Expression of the SMN gene, the spinal muscular atrophy determining gene, in the mammalian central nervous system. Hum Mol Genet 6, 1961-1971.
Boda, B., Mas, C., Giudicelli, C., Nepote, V., Guimiot, F., Levacher, B., Zvara, A., Santha, M., LeGall, I., and Simonneau, M. (2004). Survival motor neuron SMN1 and SMN2 gene promoters: identical sequences and differential expression in neurons and non-neuronal cells. Eur J Hum Genet 12, 729-737.
Burghes, A. H., and Beattie, C. E. (2009). Spinal muscular atrophy: why do low levels of survival motor neuron protein make motor neurons sick? Nat Rev Neurosci 10, 597-609.
Cherry, J. J., and Androphy, E. J. (2012). Therapeutic strategies for the treatment of spinal muscular atrophy. Future Med Chem 4, 1733-1750.
Chiriboga, C. A., Swoboda, K. J., Darras, B. T., Iannaccone, S. T., Montes, J., De Vivo, D. C., Norris, D. A., Bennett, C. F., and Bishop, K. M. (2016). Results from a phase 1 study of nusinersen (ISIS-SMNRx) in children with spinal muscular atrophy. Neurology 86, 890-897.
Cifuentes-Rojas, C., Hernandez, A. J., Sarma, K., and Lee, J. T. (2014). Regulatory interactions between RNA and polycomb repressive complex 2. Mol Cell 55, 171-185.
Davidovich, C., and Cech, T. R. (2015). The recruitment of chromatin modifiers by long noncoding RNAs: lessons from PRC2. Rna 21, 2007-2022.
Davidovich, C., Wang, X., Cifuentes-Rojas, C., Goodrich, K. J., Gooding, A. R., Lee, J. T., and Cech, T. R. (2015). Toward a consensus on the binding specificity and promiscuity of PRC2 for RNA. Mol Cell 57, 552-558.
Di Croce, L., and Helin, K. (2013). Transcriptional regulation by Polycomb group proteins. Nat Struct Mol Biol 20, 1147-1155.
Feldkotter, M., Schwarzer, V., Wirth, R., Wienker, T. F., and Wirth, B. (2002). Quantitative analyses of SMN1 and SMN2 based on real-time lightCycler PCR: fast and highly reliable carrier testing and prediction of severity of spinal muscular atrophy. Am J Hum Genet 70, 358-368.
Hua, Y., Sahashi, K., Hung, G., Rigo, F., Passini, M. A., Bennett, C. F., and Krainer, A. R. (2010). Antisense correction of SMN2 splicing in the CNS rescues necrosis in a type III SMA mouse model. Genes Dev 24, 1634-1644.
Kadoch, C., Copeland, R. A., and Keilhack, H. (2016). PRC2 and SWI/SNF Chromatin Remodeling Complexes in Health and Disease. Biochemistry 55, 1600-1614.
Kauppinen, S., Vester, B., and Wengel, J. (2005). Locked nucleic acid (LNA): High affinity targeting of RNA for diagnostics and therapeutics. Drug Discov Today Technol 2, 287-290.
Kotake, Y., Nakagawa, T., Kitagawa, K., Suzuki, S., Liu, N., Kitagawa, M., and Xiong, Y. (2011). Long non-coding RNA ANRIL is required for the PRC2 recruitment to and silencing of p15(INK4B) tumor suppressor gene. Oncogene 30, 1956-1962.
Le, T. T., Pham, L. T., Butchbach, M. E., Zhang, H. L., Monani, U. R., Coovert, D. D., Gavrilina, T. O., Xing, L., Bas sell, G. J., and Burghes, A. H. (2005). SMNDelta7, the major product of the centromeric survival motor neuron (SMN2) gene, extends survival in mice with spinal muscular atrophy and associates with full-length SMN. Hum Mol Genet 14, 845-857.
Lefebvre, S., Burlet, P., Liu, Q., Bertrandy, S., Clermont, O., Munnich, A., Dreyfuss, G., and Melki, J. (1997). Correlation between severity and SMN protein level in spinal muscular atrophy. Nat Genet 16, 265-269.
Luco, R. F., Pan, Q., Tominaga, K., Blencowe, B. J., Pereira-Smith, O. M., and Misteli, T. (2010). Regulation of alternative splicing by histone modifications. Science 327, 996-1000.
Margueron, R., Li, G., Sarma, K., Blais, A., Zavadil, J., Woodcock, C. L., Dynlacht, B. D., and Reinberg, D. (2008). Ezh1 and Ezh2 maintain repressive chromatin through different mechanisms. Mol Cell 32, 503-518.
Monani, U. R. (2005). Spinal muscular atrophy: a deficiency in a ubiquitous protein; a motor neuron-specific disease. Neuron 48, 885-896.
Palacino, J., Swalley, S. E., Song, C., Cheung, A. K., Shu, L., Zhang, X., Van Hoosear, M., Shin, Y., Chin, D. N., Keller, C. G., et al. (2015). SMN2 splice modulators enhance U1-pre-mRNA association and rescue SMA mice. Nat Chem Biol 11, 511-517.
Rossoll, W., Kroning, A. K., Ohndorf, U. M., Steegborn, C., Jablonka, S., and Sendtner, M. (2002). Specific interaction of Smn, the spinal muscular atrophy determining gene product, with hnRNP-R and gry-rbp/hnRNP-Q: a role for Smn in RNA processing in motor axons? Hum Mol Genet 11, 93-105.
Simon, J. A., and Kingston, R. E. (2013). Occupying chromatin: Polycomb mechanisms for getting to genomic targets, stopping transcriptional traffic, and staying put. Mol Cell 49, 808-824.
Sing, A., Pannell, D., Karaiskakis, A., Sturgeon, K., Djabali, M., Ellis, J., Lipshitz, H. D., and Cordes, S. P. (2009). A vertebrate Polycomb response element governs segmentation of the posterior hindbrain. Cell 138, 885-897.
Vitte, J., Fassier, C., Tiziano, F. D., Dalard, C., Soave, S., Roblot, N., Brahe, C., Saugier-Veber, P., Bonnefont, J. P., and Melki, J. (2007). Refined characterization of the expression and stability of the SMN gene products. Am J Pathol 171, 1269-1280.
Vizan, P., Beringer, M., Ballare, C., and Di Croce, L. (2015). Role of PRC2-associated factors in stem cells and disease. Febs J 282, 1723-1735.
Woo, C. J., Kharchenko, P. V., Daheron, L., Park, P. J., and Kingston, R. E. (2010). A region of the human HOXD cluster that confers polycomb-group responsiveness. Cell 140, 99-110.
Woo, C. J., Kharchenko, P. V., Daheron, L., Park, P. J., and Kingston, R. E. (2013). Variable requirements for DNA-binding proteins at polycomb-dependent repressive regions in human HOX clusters. Mol Cell Biol 33, 3274-3285.
Zhang, E. B., Yin, D. D., Sun, M., Kong, R., Liu, X. H., You, L. H., Han, L., Xia, R., Wang, K. M., Yang, J. S., et al. (2014). P53-regulated long non-coding RNA TUG1 affects cell proliferation in human non-small cell lung cancer, partly through epigenetically regulating HOXB7 expression. Cell Death Dis 5, e1243.
Zhao, J., Ohsumi, T. K., Kung, J. T., Ogawa, Y., Grau, D. J., Sarma, K., Song, J. J., Kingston, R. E., Borowsky, M., and Lee, J. T. (2010). Genome-wide identification of polycomb-associated RNAs by RIP-seq. Mol Cell 40, 939-953.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, e.g., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, e.g., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (e.g. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, e.g., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

### Certain Sequences of the Disclosure

>SEQ ID NO: 1-SMN AS 4263 region EMSA probe
(441 nt)
TCCCCTAAACAAAGACGAGGTCTTGCTATGTTGCCCAGACTGGTCTCAA
ACTCCTGAGCTCAAGTGATCCTCCTGCCTCAGCCTTCTAAAATGCCGGG
ATTACAGGCATGAGCCACTGTACCTGGCCTTAAATTTCTTAACATAGCT
AGCATTTGGAGAAAACCAACCAATAACAACAAAAGACCAACAAAATTAA
ATTTAACGAGGACGAAAAGACAGCAAGTGACATAAAAAGTTTAAACATT
TTGATTTAGACTATGTATCTGTTCCACTATGAAGCTATGAGTAAAAAAA
AAAAATCAAGCATAAATACTTTCATGCTTTTCCTTAATACACACACACA
CACACACACACACACAGCTCACATAGCATTTCGAGGGCGATTTTAAGTA
AATGTCTTGGGTAGAACACCTGTTCTAACCCCATCCCAATACACAGTAT
(Oligo 63 is complementary to the bold,
underlined sequence.)
>SEQ ID NO: 2-SMN AS neg. control (441 nt)
AAAATCAGCCCCCTGAGACCAATGAAGCATGATGTATATATTCTAAGAG
GGTACATCATTTTAGATTCAAGAAACTGTAATATAATGTAAGCCTCAAT
AAGAACATTATCACAGAAAATCTTAAAACTTTTGGTGAGTCATGCTTGT
TTTTTGAAAATGACTGCCTAGGCTAGGCGCCTGTAATCCTAGCACTTTG
GAAGGCCAAGGTGGGGAGATCACTTGAGGTCAGGAGTTCAAGACCAGCC
TGGCCAACATGGTGAAACCCCATCTCTACTAAAAATACAAAATAAGCCG
GGTGTGGTGGCGGGTGCCTGTAATCTCAGCTACTTGGGAGGCTGAAGCA
GGAGAATCACTTGAACCCAGGAGGTGGAAGTTGCAGTGAGCTGAAATGG
TGCCACTGCACTCCAGCCTGAGCGACAGAGCAAGACTCCATCTCGAAAA
>SEQ ID NO: 3-MBP 1-441 nt
GAAGAAGGTAAACTGGTAATCTGGATTAACGGCGATAAAGGCTATAACG
GTCTCGCTGAAGTCGGTAAGAAATTCGAGAAAGATACCGGAATTAAAGT
CACCGTTGAGCATCCGGATAAACTGGAAGAGAAATTCCCACAGGTTGCG
GCAACTGGCGATGGCCCTGACATTATCTTCTGGGCACACGACCGCTTTG
GTGGCTACGCTCAATCTGGCCTGTTGGCTGAAATCACCCCGGACAAAGC
GTTCCAGGACAAGCTGTATCCGTTTACCTGGGATGCCGTACGTTACAAC
GGCAAGCTGATTGCTTACCCGATCGCTGTTGAAGCGTTATCGCTGATTT
ATAACAAAGATCTGCTGCCGAACCCGCCAAAAACCTGGGAAGAGATCCC
GGCGCTGGATAAAGAACTGAAAGCGAAAGGTAAGAGCGCGCTGATGTTC
>SEQ ID NO: 4-Oligo 63: CATAGUGGAACAGAT
>SEQ ID NO: 5-Oligo 52: AGATGCAGTGCTCTT

What is claimed is:

1. A method of producing a steric-blocking oligonucleotide, the method comprising:
determining that a non-coding RNA scaffold has a first interaction region that interacts with a repressor of a target gene and a second interaction region that interacts with an activator of the target gene; and
producing a steric-blocking oligonucleotide having a region of complementarity that is complementary with the first interaction region or the second interaction region.

2. The method of claim 1, wherein the steric-blocking oligonucleotide is complementary with the first interaction region and selectively inhibits interaction of the repressor with the non-coding RNA scaffold.

3. The method of claim 1 or 2, wherein the steric-blocking oligonucleotide is complementary with the second interaction region and selectively inhibits interaction of the activator with the non-coding RNA scaffold.

4. The method of claim 1, wherein the repressor is a Polycomb Repressive Complex or a subunit thereof.

5. The method of claim 4, wherein the repressor is Polycomb Repressive Complex 1 or 2.

6. The method of claim 4, wherein the repressor is SUZ12, EZH2, EED, AEBP2, JARID2, PCL, RbAp46/48, or EZH1.

7. The method of claim 1, wherein the activator is a histone methyltransferase.

8. The method of claim 7, wherein the activator is SETD2.

9. The method of any one of claims 1 to 8, wherein the region of complementarity is at least 8 contiguous nucleotides in length.

10. The method of any one of claims 1 to 8, wherein the region of complementarity is in a range of 8 to 20 nucleotides in length.

11. The method of any one of claims 1 to 8, wherein the steric-blocking oligonucleotide is between 8 and 20 nucleotides in length and wherein the region of complementarity is in a range of 8 to 20 nucleotides in length.

12. The method of any one of claims 1 to 11, wherein the steric-blocking oligonucleotide is a mixmer.

13. The method of any one of claims 1 to 12, wherein the non-coding RNA scaffold is expressed from a chromosomal locus containing the target gene.

14. The method of any one of claims 1 to 13, wherein the steric-blocking oligonucleotide modulates expression of the target gene when delivered to a cell containing the target gene.

15. A method of preparing a steric-blocking oligonucleotide, the method comprising:
determining that a non-coding RNA scaffold interacts with an activator of the target gene and a repressor of the target gene;
identifying an interaction region of the non-coding RNA that interacts with either the activator or the repressor, but not both; and
preparing a steric-blocking oligonucleotide having a region of complementarity that is complementary with the interaction region.

16. A method of modulating expression of a target gene in a cell, the method comprising:
delivering to the cell an effective amount of a steric-blocking oligonucleotide, wherein the cell expresses a non-coding RNA scaffold, wherein prior to delivering the steric-blocking oligonucleotide it has been determined that the non-coding RNA scaffold has a first interaction region that interacts with a repressor of the target gene and a second interaction region that interacts with an activator of the target gene, and wherein the steric-blocking oligonucleotide has a region of complementarity that is complementary with the first interaction region or the second interaction region.

17. The method of claim 16, wherein the steric-blocking oligonucleotide is complementary with the first interaction region and selectively inhibits interaction of the repressor with the non-coding RNA scaffold.

18. The method of claim 16, wherein the steric-blocking oligonucleotide is complementary with the second interaction region and selectively inhibits interaction of the activator with the non-coding RNA scaffold.

19. The method of any one of claims 16 to 18, wherein the target gene is an SMN gene.

20. The method of claim 16, wherein the repressor is a Polycomb Repressive Complex 2 subunit.

21. The method of claim 20, wherein the repressor is SUZ12, EZH2, EED, AEBP2, JARID2, PCL, RbAp46/48, or EZH1.

22. The method of claim 16, wherein the activator is a hi stone methyltransferase.

23. The method of claim 22, wherein the activator is SETD2.

24. The method of any one of claims 16 to 23, wherein the region of complementarity is at least 8 contiguous nucleotides in length.

25. The method of any one of claims 16 to 24, wherein the region of complementarity is in a range of 8 to 20 nucleotides in length.

26. The method of any one of claims 16 to 24, wherein the steric-blocking oligonucleotide is between 8 and 20 nucleotides in length and wherein the region of complementarity is in a range of 8 to 20 nucleotides in length.

27. The method of any one of claims 16 to 26, wherein the steric-blocking oligonucleotide is a mixmer.

28. The method of any one of claims 16 to 27, wherein the cell is in vivo.

29. The method of any one of claims 16 to 28, wherein the cell is in vitro.

30. The method of any one of claims 16 to 29, wherein the non-coding RNA scaffold is expressed from a chromosomal locus containing the target gene.

31. A method of modulating expression of a target gene in a cell, wherein it has been determined that a non-coding RNA interacts with both an activator of the target gene and a repressor of a target gene, the method comprising:
delivering to the cell a steric-blocking oligonucleotide having a region of complementarity that is complementary with a region of the non-coding RNA that interacts with either the activator or the repressor, but not both.

32. A method of increasing expression of a target gene in a cell, the method comprising:
delivering to the cell an effective amount of a steric-blocking oligonucleotide, wherein the cell expresses a non-coding RNA, wherein prior to delivering the steric-blocking oligonucleotide it has been determined that the non-coding RNA scaffold has a first interaction region that interacts with a repressor of the target gene and a second interaction region that interacts with an activator of the target gene, wherein the steric-blocking oligonucleotide has a region of complementarity that is complementary with the first interaction region.

33. The method of claim 32, wherein displacement of the repressor from the first interaction region indicates effectiveness of the steric-blocking oligonucleotide.

34. The method of claim 32, wherein the steric-blocking oligonucleotide is complementary with the first interaction region and selectively inhibits interaction of the repressor with the non-coding RNA scaffold.

35. The method of any one of claims 32 to 34, wherein the target gene is an SMN gene.

36. The method of claim 32, wherein the repressor is a Polycomb Repressive Complex 2 subunit.

37. The method of claim 36, wherein the repressor is SUZ12, EZH2, EED, AEBP2, JARID2, PCL, RbAp46/48, or EZH1.

38. The method of claim 32, wherein the activator is a histone methyltransferase.

39. The method of claim 38, wherein the activator is SETD2.

40. The method of any one of claims 32 to 39, wherein the region of complementarity is at least 8 contiguous nucleotides in length.

41. The method of any one of claims 32 to 39, wherein the region of complementarity is in a range of 8 to 20 nucleotides in length.

42. The method of any one of claims 32 to 41, wherein the steric-blocking oligonucleotide is between 8 and 20 nucleotides in length and wherein the region of complementarity is in a range of 8 to 20 nucleotides in length.

43. The method of any one of claims 32 to 42, wherein the steric-blocking oligonucleotide is a mixmer.

44. The method of any one of claims 32 to 43, wherein the cell is in vivo.

45. The method of any one of claims 32 to 43, wherein the cell is in vitro.

46. The method of any one of claims 32 to 45, wherein the non-coding RNA scaffold is expressed from a chromosomal locus containing the target gene.

47. A method of increasing expression of a target gene in a cell, the method comprising:
delivering to the cell a steric-blocking oligonucleotide having a region of complementarity that is complementary with a region of the non-coding RNA scaffold that interacts with a repressor of the target gene, wherein displacement of the repressor from the non-coding RNA, without displacement an activator of the target gene that also interacts with the non-coding RNA scaffold, indicates effectiveness of the steric-blocking oligonucleotide.


SEQUENCE LISTING (53 sequences)

SEQ ID NO: 1 - DNA, 441 bp, Artificial Sequence
  SMN AS 4263 region EMSA probe
tcccctaaacaaagacgaggtcttgctatgttgcccagactggtctcaaactcctgagctcaagtgatcctcctgcctcagccttctaaaatgccgggattacaggcatgagccactgtacctggccttaaatttcttaacatagctagcatttggagaaaaccaaccaataacaacaaaagaccaacaaaattaaatttaacgaggacgaaaagacagcaagtgacataaaaagtttaaacattttgatttagactatgtatctgttccactatgaagctatgagtaaaaaaaaaaaatcaagcataaatactttcatgcttttccttaatacacacacacacacacacacacacacagctcacatagcatttcgagggcgattttaagtaaatgtcttgggtagaacacctgttctaaccccatcccaatacacagtat

SEQ ID NO: 2 - DNA, 441 bp, Artificial Sequence
  SMN AS neg. control
aaaatcagccccctgagaccaatgaagcatgatgtatatattctaagagggtacatcattttagattcaagaaactgtaatataatgtaagcctcaataagaacattatcacagaaaatcttaaaacttttggtgagtcatgcttgttttttgaaaatgactgcctaggctaggcgcctgtaatcctagcactttggaaggccaaggtggggagatcacttgaggtcaggagttcaagaccagcctggccaacatggtgaaaccccatctctactaaaaatacaaaataagccgggtgtggtggcgggtgcctgtaatctcagctacttgggaggctgaagcaggagaatcacttgaacccaggaggtggaagttgcagtgagctgaaatggtgccactgcactccagcctgagcgacagagcaagactccatctcgaaaa

SEQ ID NO: 3 - DNA, 441 bp, Artificial Sequence
  MBP 1-441 nt
gaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttc

SEQ ID NO: 4 - DNA, 15 bp, Artificial Sequence
  Synthetic polynucleotide
cataguggaacagat

SEQ ID NO: 5 - DNA, 15 bp, Artificial Sequence
  Synthetic polynucleotide
agatgcagtgctctt

SEQ ID NO: 6 - DNA, 15 bp, Artificial Sequence
  Synthetic polynucleotide
agaugcagtgcucut

SEQ ID NO: 7 - DNA, 15 bp, Artificial Sequence
  Synthetic polynucleotide
cataguggaacagat

SEQ ID NO: 8 - DNA, 18 bp, Artificial Sequence
  Synthetic polynucleotide
tcactttcataatgctgg

SEQ ID NO: 9 - DNA, 15 bp, Artificial Sequence
  Synthetic polynucleotide
catagtggaacagat

SEQ ID NO: 10 - DNA, 40 bp, Artificial Sequence
  Synthetic polynucleotide
taatacgactcactatagtcccctaaacaaagacgaggtc

SEQ ID NO: 11 - DNA, 22 bp, Artificial Sequence
  Synthetic polynucleotide
atactgtgtattgggatggggt

SEQ ID NO: 12 - DNA, 40 bp, Artificial Sequence
  Synthetic polynucleotide
taatacgactcactatagaaaatcagccccctgagaccaa

SEQ ID NO: 13 - DNA, 24 bp, Artificial Sequence
  Synthetic polynucleotide
ttttcgagatggagtcttgctctg

SEQ ID NO: 14 - DNA, 46 bp, Artificial Sequence
  Synthetic polynucleotide
taatacgactcactatagattgtttatatattcttgcccatcgggg

SEQ ID NO: 15 - DNA, 68 bp, Artificial Sequence
  Synthetic polynucleotide
cacaaaaccatatttccatccaccaagctaatacgactcactatagatgaaaataaaaacaggtgcac

SEQ ID NO: 16 - DNA, 29 bp, Artificial Sequence
  Synthetic polynucleotide
cagatctttgttataaatcagcgataacg

SEQ ID NO: 17 - DNA, 20 bp, Artificial Sequence
  Synthetic polynucleotide
gcagtgctcttgtagtccca

SEQ ID NO: 18 - DNA, 21 bp, Artificial Sequence
  Synthetic polynucleotide
cctccttatggcatagacacc

SEQ ID NO: 19 - DNA, 21 bp, Artificial Sequence
  Synthetic polynucleotide
cctccttatggcatagacacc

SEQ ID NO: 20 - DNA, 24 bp, Artificial Sequence
  Synthetic polynucleotide
cttctgccaggaaagaaggcaacc

SEQ ID NO: 21 - DNA, 24 bp, Artificial Sequence
  Synthetic polynucleotide
gctgatgctttgggaagtatgtta

SEQ ID NO: 22 - DNA, 25 bp, Artificial Sequence
  Synthetic polynucleotide
caccttccttctttttgattttgtc

SEQ ID NO: 23 - DNA, 21 bp, Artificial Sequence
  Synthetic polynucleotide
tacatgagtggctatcatact

SEQ ID NO: 24 - DNA, 20 bp, Artificial Sequence
  Synthetic polynucleotide
tggaccaccaataattcccc

SEQ ID NO: 25 - DNA, 25 bp, Artificial Sequence
  Synthetic polynucleotide
atgccagcatttccatataatagcc

SEQ ID NO: 26 - DNA, 20 bp, Artificial Sequence
  Synthetic polynucleotide
tccagattctcttgatgatg

SEQ ID NO: 27 - DNA, 25 bp, Artificial Sequence
  Synthetic polynucleotide
catttgtgaaacttcgggtaaacca

SEQ ID NO: 28 - DNA, 28 bp, Artificial Sequence
  Synthetic polynucleotide
gtaaggaagctgcagtattcttcttttg

SEQ ID NO: 29 - DNA, 18 bp, Artificial Sequence
  Synthetic polynucleotide
cacacctaaaagaaaacc

SEQ ID NO: 30 - DNA, 24 bp, Artificial Sequence
  Synthetic polynucleotide
tttacccagctaccattgcttcaa

SEQ ID NO: 31 - DNA, 25 bp, Artificial Sequence
  Synthetic polynucleotide
cggacagattttgctcctctctatt

SEQ ID NO: 32 - DNA, 18 bp, Artificial Sequence
  Synthetic polynucleotide
acctgtgttgtggtttac

SEQ ID NO: 33 - DNA, 26 bp, Artificial Sequence
  Synthetic polynucleotide
ccttctggaccaccagtaagtaaaaa

SEQ ID NO: 34 - DNA, 30 bp, Artificial Sequence
  Synthetic polynucleotide
gggatgttctacaatgacattttacaatcc

SEQ ID NO: 35 - DNA, 20 bp, Artificial Sequence
  Synthetic polynucleotide
ttgctttcacatacaatttg

SEQ ID NO: 36 - DNA, 25 bp, Artificial Sequence
  Synthetic polynucleotide
atcactcagcatcttttcctgacaa

SEQ ID NO: 37 - DNA, 45 bp, Artificial Sequence
  Synthetic polynucleotide
gcctcagacagttgtatttttttatttttattttttagtaatata

SEQ ID NO: 38 - DNA, 24 bp, Artificial Sequence
  Synthetic polynucleotide
atgtgactttgttttgtaaattta

SEQ ID NO: 39 - DNA, 30 bp, Artificial Sequence
  Synthetic polynucleotide
aaaatgtcttgtgaaacaaaatgcttttta

SEQ ID NO: 40 - DNA, 30 bp, Artificial Sequence
  Synthetic polynucleotide
ccttctttttgattttgtctgaaacctgta

SEQ ID NO: 41 - DNA, 26 bp, Artificial Sequence
  Synthetic polynucleotide
aaaataaaggaagttaaaaaaaatag

SEQ ID NO: 42 - DNA, 18 bp, Artificial Sequence
  Synthetic polynucleotide
cggtggtgaggcagttga

SEQ ID NO: 43 - DNA, 32 bp, Artificial Sequence
  Synthetic polynucleotide
cccttctcacagctcataaaattaccaataat

SEQ ID NO: 44 - DNA, 19 bp, Artificial Sequence
  Synthetic polynucleotide
aatccacattcaaattttc

SEQ ID NO: 45 - DNA, 23 bp, Artificial Sequence
  Synthetic polynucleotide
tcccattttgtaggttgcctgtt

SEQ ID NO: 46 - DNA, 24 bp, Artificial Sequence
  Synthetic polynucleotide
actaaagagcttctgcacagcaaa

SEQ ID NO: 47 - DNA, 19 bp, Artificial Sequence
  Synthetic polynucleotide
cactctgatggtagtttct

SEQ ID NO: 48 - DNA, 24 bp, Artificial Sequence
  Synthetic polynucleotide
cagcctcctgagtagctaggatta

SEQ ID NO: 49 - DNA, 26 bp, Artificial Sequence
  Synthetic polynucleotide
ggtgaaaccccgtctctactaaaaaa

SEQ ID NO: 50 - DNA, 17 bp, Artificial Sequence
  Synthetic polynucleotide
caggcacacgccaccat

SEQ ID NO: 51 - DNA, 23 bp, Artificial Sequence
  Synthetic polynucleotide
gagacttcagcagtcacagtgat

SEQ ID NO: 52 - DNA, 19 bp, Artificial Sequence
  Synthetic polynucleotide
ggaggagagcgctgtaact

SEQ ID NO: 53 - DNA, 16 bp, Artificial Sequence
  Synthetic polynucleotide
tccggtgcacatccta